(19) United States
(12) Patent Application Publication
(10) Pub. No.: US 2026/0071181 A1
(43) Pub. Date: Mar. 12, 2026

(54) UNIVERSAL NATURAL KILLER CELLS DERIVED FROM HUMAN PLURIPOTENT STEM CELLS AND METHOD OF USE

(71) Applicant: Purdue Research Foundation, West Lafayette, IN (US)

(72) Inventors: Xiaoping Bao, West Lafayette, IN (US); Philip Stewart Low, West Lafayette, IN (US); Yun Chang, West Lafayette, IN (US); Juhyung Jung, West Lafayette, IN (US)

(21) Appl. No.: 19/104,354

(22) PCT Filed: Aug. 16, 2023
(86) PCT No.: PCT/US2023/072341

§ 371 Date: Feb. 17, 2025

Related U.S. Application Data
(60) Provisional application No. 63/398,781, filed on Aug. 17, 2022.

(51) Int. Cl. C12N 5/0783; A61K 35/17; A61K 40/15; A61K 40/31; A61K 40/42; A61K 45/06; A61P 35/00; C07K 14/47

(52) U.S. Cl. CPC C12N 5/0646; A61K 35/17; A61K 40/15; A61K 40/31; A61K 40/42; A61K 45/06; A61P 35/00; C07K 14/4702; C07K 2317/622; C07K 2319/03; C12N 2506/02; C12N 2506/45

(57) ABSTRACT

A population of universal natural killer (NK) cells derived from human pluripotent stem cells (hPSCs) and engineered to overexpress the transcription factor ID2, NFIL3, and/or SPI1 and, optionally, an anti-programmed death ligand 1 (PD-L1) chimeric antigen receptor (CAR) and an anti-fluorescein isothiocyanate CAR are provided. Methods of treating cancer in a subject using the population of universal NK cells are also provided.

### PRIORITY

This application is related to and claims the priority benefit of U.S. Provisional Patent Application No. 63/398,781 filed Aug. 17, 2022. The content of the aforementioned application is hereby incorporated by reference in its entirety into this disclosure.

### SEQUENCE LISTINGS

The sequences herein (SEQ ID NOS: 1-4) are also provided in computer-readable form encoded in a file filed herewith and incorporated herein by reference. The information recorded in computer-readable form is identical to the written Sequence Listings provided herein (e.g., pursuant to the United States Code of Federal Regulations 37 C.F.R. § 1.821(f)).

### TECHNICAL FIELD

The present disclosure relates to human pluripotent stem cells (hPSCs), natural killer (NK) cells derived from hPSCs, engineered NK cells derived from hPSCs, and methods of using the NK cells, e.g., engineered NK cells, such as in the treatment of cancer.

### BACKGROUND

Natural killer (NK) cells are one kind of lymphocytes that are differentiated from hematopoietic stem cells (HSCs) in the bone marrow and matured in lymph nodes. Rohaan et al., Adoptive cellular therapies: the current landscape, Virchows Archiv 474: 449-461 (2019); Hinrichs & Rosenberg, Exploiting the curative potential of adoptive T-cell therapy for cancer, Immunological Reviews 257(1): 56-71 (2014). NK cells present characteristics of both innate and adaptive lymphoid cells, demonstrating superior ability to attack tumor cells and suppress their growth in vivo. Hinrichs & Rosenberg (2014), supra; Redeker & Arens, Improving adoptive T cell therapy: The particular role of T cell costimulation, cytokines, and post-transfer vaccination, Frontiers Immunology 7 (2016); Zhu et al., Metabolic Reprograming via Deletion of CISH in Human iPSC-Derived NK Cells Promotes In Vivo Persistence and Enhances Anti-tumor Activity, Cell Stem Cell 27(2): 224-237 (2020); Goldenson et al., Umbilical Cord Blood and iPSC-Derived Natural Killer Cells Demonstrate Key Differences in Cytotoxic Activity and KIR Profiles, Frontiers Immunology 11 (2020). Unlike T cells, prior sensitization and antigen exposure are not required for NK cells. Cichocki et al., iPSC-derived NK cells maintain high cytotoxicity and enhance in vivo tumor control in concert with T cells and anti-PD-1 therapy, Science Translational Medicine 12(568) (2020); Zhu et al., Pluripotent stem cell-derived NK cells with high-affinity noncleavable CD16a mediate improved antitumor activity, Blood 135(6): 399-410 (2020). Activating receptors, such as CD16 (FcγRIII) and NK group 2D (NKG2D), and inhibitory receptors expressed on NK cells can work synergistically to distinguish normal cells from tumor cells, triggering cytolytic programs and cytokine release against abnormal cells. Chang & Bao, Adoptive natural killer cell therapy: a human pluripotent stem cell perspective, Current Opinions Chemical Engineering 30: 69-76 (2020). Importantly, allogeneic NK cells are free of graft-versus-host diseases (GvHD) which is commonly associated with allogeneic T cell-based cancer therapies. Handgretinger et al., Exploitation of natural killer cells for the treatment of acute leukemia, Blood 127(26): 3341-3349 (2016). This unique characteristic opens the possibility to develop universal NK cells, which could be used to treat any patient without human leukocyte antigen matching.

Given their unique ability to self-renew and differentiate into all kinds of somatic cells, human pluripotent stem cells (hPSCs) are emerging as a promising cell source for scalable production of NK cells. As compared to primary NK cells or the NK-92 cell line, hPSCs are more accessible to genetic modifications, such as chimeric antigen receptor engineering, to produce potentially off-the-shelf, genetically-enhanced NK cells for cancer immunotherapy. Romee et al., Cytokine-induced memory-like natural killer cells exhibit enhanced responses against myeloid leukemia, Science Translational Med 8(357) (2016); Cerwenka & Lanier, Natural killer cell memory in infection, inflammation and cancer, Nature Reviews Immunology 16: 112-123(2016).

However, there are several obstacles to overcome before realizing the full potential of hPSC-NK cells. Paust & Von Andrian, Natural killer cell memory, Nature Immunology 12: 500-508 (2011). First, many established differentiation protocols require embryoid body formation, feeder cells, and/or stromal cells, reducing the reproducibility and standardization of massive NK cell production from hPSCs. O'Sullivan et al., Natural Killer Cell Memory, Immunity 43(4): 634-645 (2015); Sun et al., Adaptive immune features of natural killer cells, Nature 457: 557-561 (2009); Berrien-Elliott et al., Human Cytokine-Induced Memory-Like Natural Killer Cells, J Innate Immunology 7(6): 563-671 (2015); Cooper et al., Cytokine-induced memory-like natural killer cells, PNAS USA 106(6): 1915-1919 (2009); Ma et al., An oncolytic virus expressing il15/il15ra combined with off-the-shelf egfr-car nk cells targets glioblastoma, Cancer Research 81(13): 3635-3648 (2021); Liu et al., Cord blood NK cells engineered to express IL-15 and a CD19-targeted CAR show long-term persistence and potent antitumor activity, Leukemia 32: 520-531 (2018). Second, more cost-effective protocols are needed, as the heavy employment of expensive growth factors and animal-derived components in current protocols can limit the ability of NK cell manufacturing to achieve a clinically-relevant dosage (i.e., 10⁷ NK cells per kg of a patient). Du et al., piggyBac system to co-express NKG2D CAR and IL-15 to augment the in vivo persistence and anti-AML activity of human peripheral blood NK cells, Molecular Therapy—Methods & Clinical Development 23: 582-596 (2021). Notably, NK cells have a long derivation period from hPSCs of seven or more weeks, which complicates cell preparation and increases contamination risk. In view of the above, it is an object of the present disclosure to provide anew platform for efficient production of universal NK cells, which can be used in various therapies (e.g., targeted cancer immunotherapy) to treat any patient without human leukocyte antigen matching.

Furthermore, while adoptive chimeric antigen receptor (CAR)-engineered NK cells have shown some promise in treating various cancers, limited immunological memory and access to sufficient numbers of allogeneic donor cells have hindered their broader preclinical and clinical applications. The failure of transferred NK cells to develop classical immunological memory is mainly caused by the inability of receptor genes in NK cells to undergo rearrangement, and the exhaustion of NK cells under an immunosuppressive tumor microenvironment (TME). Cerwenka et al. (2016), supra; Paust (2011), supra; O'Sullivan et al. (2015), supra; Sun et al. (2009), supra. Accordingly, if achieved, engineering NK cells with TME-responsive CARs holds great promise in achieving immunological memory-like activities of NK cells during tumor ablation.

Specific receptor stimulation promotes significant expansion of NK cells under a diseased microenvironment, and these self-renewal memory NK cells rapidly degranulate and produce cytokines upon reactivation to perform robust protective immunity. Berrien-Elliott et al. (2015), supra; Cooper et al. (2009), supra. While various tumor targeting CARs in NK cells have been effectively stimulated by specific tumor antigens, their therapeutic efficacy to date remains limited due to the poor in vivo expansion and persistence of NK cells after infusion. The employment of cytokines, such as IL-15, IL-18, and IL-21, has been widely reported to enhance persistence and/or memory of various NK cells in vivo. Ma et al. (2021), supra; Liu et al. (2018); Romee et al. (2016), supra; Cooper et al. (2009), supra; Romee et al., Cytokine activation induces human memory-like NK cells, Blood 120(24): 4751-4760 (2012); Gang et al., CAR-modified memory-like NK cells exhibit potent responses to NK-resistant lymphomas, Blood 136(20): 2308-2318 (2020); Lopez-Vergès et al., CD57 defines a functionally distinct population of mature NK cells in the human CD56dimCD16+ NK-cell subset, Blood 116(19): 3865-3874 (2010); Skak et al., Interleukin-21 activates human natural killer cells and modulates their surface receptor expression, Immunology 123(4): 575-583 (2008); Heinze et al., The Synergistic Use of IL-15 and IL-21 for the Generation of NK Cells From CD3/CD19-Depleted Grafts Improves Their ex vivo Expansion and Cytotoxic Potential Against Neuroblastoma: Perspective for Optimized Immunotherapy Post Haploidentical Stem Cell Transplantation, Frontiers Immunology 10 (2019); Ma et al., Natural Killer (NK) and CAR-NK Cell Expansion Method using Membrane Bound-IL-21-Modified B Cell Line, JoVE J (2022). However, cytokine stimulation can lead to autonomous NK cell proliferation or even leukemia transformation. Mishra et al., Aberrant Overexpression of IL-15 Initiates Large Granular Lymphocyte Leukemia through Chromosomal Instability and DNA Hypermethylation, Cancer Cell 22(5): 645-655 (2012).

To produce superior memory-like NK cells under a robust, safe and controllable way, CAR structures should be designed to effectively and specifically recognize immunosuppressive signals in the TME and immediately activate intracellular proliferation signaling pathway in NK cells, leading to tumor-responsive cellular expansion and prevention of NK cell exhaustion. Liu et al. (2018), supra; Du et al. (2021), supra. Among these immunosuppressive signals, programmed death-ligand 1 (PD-L1), which are expressed on various solid tumor cells and interact with PD-1 on immune cells to block immunotherapy, are widely used in CAR design since PD-L1/PD-1 blockade has achieved significant clinical benefits. Chen et al., Exosomal PD-L1 contributes to immunosuppression and is associated with anti-PD-1 response, Nature 560: 382-386 (2018); Jiang et al., Role of the tumor microenvironment in PD-L1/PD-1-mediated tumor immune escape, Molecular Cancer 18: 10 (2019). In addition to enhanced in vivo persistence, these memory-like NK cells should have excellent tumor-killing ability. CAR constructs containing transmembrane and/or co-stimulatory domains of NKG2D, 2B4, and 41BB have been reported to effectively activate intracellular cytotoxicity signaling pathways in NK cells, but continuous exposure to antigens can cause NK cell exhaustion and prevent acquisition of memory-like phenotype in the engineered NK cells. Li et al., Human iPSC-Derived Natural Killer Cells Engineered with Chimeric Antigen Receptors Enhance Anti-tumor Activity, Cell Stem Cell 23(2): 181-192 (2018); Seo et al., IL-21-mediated reversal of NK cell exhaustion facilitates anti-Tumour immunity in MHC class I-deficient tumours, Nature Communications 8: 15776 (2017); Judge et al., Characterizing the Dysfunctional NK Cell: Assessing the Clinical Relevance of Exhaustion, Anergy, and Senescence, Frontiers in Cellular & Infection Microbiology 10 (2020).

To avoid T cell exhaustion and cytokine storm, an anti-fluorescein isothiocyanate (FITC) single-chain variable fragment (scFv)-based CAR has been used in T cells in hopes of eradicating tumor cells only in the presence of a low molecular weight adapter. Luo et al., Targeted Rejuvenation of Exhausted Chimeric Antigen Receptor T-cells Regresses Refractory Solid Tumors, Molecular Cancer Research 20(5): 823-833 (2022); Tamada et al., Redirecting gene-modified T cells toward various cancer types using tagged antibodies, Clinical Cancer Research 18(23): 6436-6445 (2012); Ma et al., Versatile strategy for controlling the specificity and activity of engineered T cells, PNAS USA 113(4): E450-E458 (2016); Lee et al., Regulation of CAR T cell-mediated cytokine release syndrome-like toxicity using low molecular weight adapters, Nature Communications 10: 2681 (2019); Lee et al., Use of a single CAR T cell and several bispecific adapters facilitates eradication of multiple antigenically different solid tumors, Cancer Research 79(2): 387-396 (2019). These fluorescein-cancer, bridging small molecules have a short circulation half-life (e.g., <90 minutes) and can easily penetrate solid tumors. Lee et al. (2019), supra. Such a bi-specific adapter strategy can also be used to prevent NK cell exhaustion and reduce off-target toxicity to the non-target organs. However, the broader application of adoptive NK cell therapy has to date been hindered by the limited access to sufficient numbers of donor cells for multiple-dose transplantation. Zhu et al., Concise Review: Human Pluripotent Stem Cells to Produce Cell-Based Cancer Immunotherapy, Stem Cells 36(2): 134-145 (2018). Additionally, genetic modification of primary NK cells is technically challenging and laborious, and can lead to heterogeneous CAR-NK cells. Carlsten & Childs, Genetic manipulation of NK cells for cancer immunotherapy: Techniques and clinical implications, Frontiers Immunology 6 (2015).

In view of the foregoing, it is another object of the present disclosure to provide NK cells with enhanced antigen-specific proliferation and anti-tumor toxicity, thereby providing universal NK cells with immunological memory-like phenotypes for targeted immunotherapy. These and other objects and advantages, as well as inventive features, will be apparent from the description provided.

### SUMMARY

Provided is a population of universal natural killer (NK) cells derived from human pluripotent stem cells (hPSCs) and engineered to overexpress the transcription factor ID2, NFIL3, and/or SPI1. The expression of the transcription factor(s) can be inducible. The majority of the NK cells can be CD45+CD56+. The NK cells can express at least one NK cell-specific marker. The at least one NK cell-specific marker can be NKp44, NKp46, KIR3DL1, NKG2D, or any combination thereof. The population of universal NK cells can be further engineered to express an anti-programmed death ligand 1 (PD-L1) chimeric antigen receptor (CAR) and an anti-fluorescein isothiocyanate (FITC) CAR. The anti-PD-L1 CAR or anti-FITC CAR can comprise a truncated cytoplasmic domain from interleukin-2 (IL-2) receptor β-chain, a STAT3-binding tyrosine-X-X-glutamine (YXXQ) motif, or both.

In certain embodiments, the population of NK cells is derived from hPSCs and engineered to: overexpress the transcription factor ID2, NFIL3, and/or SPI1; and express an anti-PD-L1 CAR and an anti-FITC CAR. The NK cells can be, for example, engineered to overexpress the transcription factor ID2. The anti-PD-L1 CAR and/or the anti-FITC CAR can comprise a truncated cytoplasmic domain from IL-2 receptor β-chain, a STAT3-binding tyrosine-X-X-glutamine (YXXQ) motif, or both.

In certain embodiments, the anti-PD-L1 CAR and/or the anti-FITC CAR comprises an NK cell-Fc receptor transmembrane domain and an intracellular signaling domain. The NK cell-Fc receptor transmembrane and intracellular signaling domains can comprise a γ-chain from CD32a or a γ-chain from CD16. The overexpression of the transcription factor(s) can be inducible. The majority of the NK cells can be CD45+CD56+. The NK cells can express at least one NK cell-specific marker. In certain embodiments, the at least one NK cell-specific marker is NKp44, NKp46, KIR3DL1, NKG2D, or any combination thereof.

The hPSCs can comprise human embryonic stem cells (hESCs) and/or induced pluripotent stem cells (iPSCs).

Also provided is a population of hPSCs engineered to express an anti-PD-L1 CAR and an anti-FITC CAR. The population of hPSCs can be further engineered to overexpress the transcription factor ID2, NFIL3, and/or SPI1. The hPSCs can comprise hESCs and/or iPSCs. In certain embodiments, the population of hPSCs is engineered to overexpress the transcription factor ID2. In certain embodiments, the overexpression of the transcription factor(s) is inducible.

In certain embodiments, the anti-PD-L1 CAR and/or the anti-FITC CAR of the population of hPSCs comprises a truncated cytoplasmic domain from IL-2 receptor β-chain, a STAT3-binding tyrosine-X-X-glutamine (YXXQ) motif, or both. In certain embodiments, the anti-PD-L1 CAR and/or the anti-FITC CAR of the population of hPSCs comprises an NK cell-Fc receptor transmembrane and intracellular signaling domains. In certain embodiments, the NK cell-Fc receptor transmembrane and intracellular signaling domains comprises a γ-chain from CD32a or a γ-chain from CD16.

CAR constructs are also provided. In certain embodiments, a CAR construct comprises one or more sequences that encode: an anti-FITC polypeptide or an anti-PD-L1 polypeptide, a NKG2d transmembrane domain, and a 2B4 co-stimulatory domain. The CAR construct can further comprise one or more sequences that encode a truncated cytoplasmic domain from IL-2 receptor β-chain, a STAT3-binding tyrosine-X-X-glutamine (YXXQ) motif, or both. The CAR construct can further comprise one or more sequences that encode FcγRIII.

Pharmaceutical compositions are also provided. In certain embodiments, a pharmaceutical composition hereof comprises any of the NK cells hereof or any of the universal NK cells hereof; and a pharmaceutically acceptable carrier and/or diluent. The pharmaceutical composition can further comprise pharmaceutically acceptable excipient.

Uses of the NK cells hereof, the constructs hereof the universal NK cells hereof, or a pharmaceutical composition hereof in the manufacture of a medicament for the treatment of cancer in a subject are also provided.

Also provided is a method of treating cancer in a subject. The method comprises administering to the subject an above-described population of universal NK cells. In certain embodiments, the method of treating cancer in a subject comprises administering to the subject a first therapy comprising a therapeutically effective amount of a population of any of the NK cells hereof, a population of NK cells expressing one or more constructs described herein; a population of universal NK cells hereof; or a pharmaceutical composition described herein, whereupon the subject is treated for cancer.

Administering the first therapy can comprise a delivery route selected from the group consisting of intravenous, intraperitoneal, intramuscular, intradermal, subcutaneous, intrathecal, intraosseous, and a combination of any of the foregoing.

The method can further comprise administering to the subject a conjugate. The conjugate can comprise FITC linked to a ligand that binds a folate receptor α (FRα). Alternatively, the conjugate can comprise FITC linked to a ligand that binds prostate-specific membrane antigen (PSMA). The ligand that binds PSMA can be DUPA. Also, alternatively, the conjugate can comprise FITC linked to a ligand that binds carbonic anhydrase IX (CAIX).

The method can further comprise administering a second therapy to the subject. The second therapy can comprise a therapeutically effective amount of chemotherapy. The second therapy can comprise a therapeutically effective amount of radiotherapy. The second therapy can comprise surgical removal of cancerous cells from the subject. The second therapy can comprise a chemotherapy, radiotherapy, or both.

The method can further comprise imaging a cancer in the subject prior to or during administration of the first and/or second therapies. The first and second therapies can be administered sequentially and/or alternatively.

Methods of producing a population of NK cells described herein are also provided. Such methods can comprise differentiating a population of hPSCs to NK cells, the population of hPSCs engineered to overexpress transcription factor ID2, NFIL3, and/or SPI1. In certain embodiments, the population of hPSCs can be engineered to express an anti-PD-L1 CAR and an anti-FITC CAR. The hPSCs can comprise hESCs and/or iPSCs. The population of hPSCs can be engineered to overexpress the transcription factor ID2. The anti-PD-L1 CAR and/or the anti-FITC CAR can comprise a truncated cytoplasmic domain from interleukin-2 (IL-2) receptor β-chain, a STAT3-binding tyrosine-X-X-glutamine (YXXQ) motif, or both. The anti-PD-L1 CAR and/or the anti-FITC CAR can comprise NK cell-Fc receptor transmembrane and intracellular signaling domains. The NK cell-Fc receptor transmembrane and intracellular signaling domains can comprise a γ-chain from CD32a or a γ-chain from CD16. The overexpression of the transcription factor(s) can be inducible.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1A is a schematic of an all-in-one, Tet-on 3G inducible system construct.

FIG. 1B is a schematic of a targeted knocked-in strategy at the endogenous AAVS1 safe harbor locus via CRISPR/Cas9-mediated homologous recombination.

FIG. 1C shows polymerase chain reaction (PCR) genotyping of human pluripotent stem cell (hPSC) clones after puromycin selection. The expected PCR product for a correctly targeted AAVS1 site is 991 bp (arrow on left). A homozygosity assay was performed on the knock-in clones, and those without ˜204 bp PCR products were homozygous (arrow on right).

FIG. 1D shows flow cytometry analysis of OCT4 and SSEA4 expression in the indicated hPSC lines.

FIG. 1E shows RT-PCR analysis of NFIL3, SPI1, and ID2 expression in indicated human pluripotent stem cell (hPSC) lines with or without doxycycline (dox) treatment.

FIG. 2A is a schematic of natural killer (NK) cell differentiation from hPSCs with or without doxycycline (dox) treatment.

FIG. 2B is a representative flow cytometry analysis of CD45 and CD56 in day-30 NK cell differentiation cultures from the indicated hPSC lines.

FIG. 2C is a bar graph showing the quantification of CD45+CD56+(%) expression for the indicated hPSC lines, with A labeling groups that did not receive dox treatment, and B labeling dox treatment groups. Three wells for each condition; data presented as mean±s.d. of three independent replicates, *p<0.05.

FIG. 3A is a schematic of NK cell differentiation from hPSCs with stage-specific overexpression of ID2 via dox treatment.

FIG. 3B is a representative flow cytometry analysis of CD45 and CD56 expression in day-30 NK cell differentiation cultures with the indicated dox treatment.

FIG. 3C is a bar graph showing the quantification of CD45+CD56+ (%) expression for the indicated hPSC lines. Three wells for each condition; data presented as mean±s.d. of three independent replicates, *p<0.05.

FIG. 3D is a representative histogram plot of the indicated NK cell markers and corresponding isotype controls (controls labeled A and stain samples labeled B).

FIG. 4A shows the expansions of the indicated NK cells at day 5 and day 15.

FIG. 4B is a schematic of an in vitro transwell model for a transmigration study.

FIG. 4C is a bar graph showing the quantification of transmigration (%) for the indicated transmigrated NK cells.

FIG. 4D shows representative images of polarized F-actin accumulation at the interface between the indicated NK cells and targeted U87MG glioblastoma cells. Scale bar, 25 μm.

FIG. 4E shows representative flow cytometry analysis of interferon γ (IFNγ)/CD107a in 1D2-hPSC-derived, wild-type hPSC-derived NK cells and peripheral blood (PB) NK cells with or without glioblastoma cell stimulation.

FIG. 4F is a bar graph showing the quantification of IFNγ+CD107a+ (%) for the indicated cells. Five replicates for each condition; data are presented as mean±s.d. of five independent replicates.

FIG. 4G shows the quantification of the cytotoxicity of ID2-hPSC-derived NK cells against the indicated tumor cells at ratios of 0:1, 3:1, 5:1, and 10:1.

FIG. 4H shows the quantification of the cytotoxicity of wild-type hPSC-derived NK cells against the indicated tumor cells at ratios of 0:1, 3:1, 5:1, and 10:1.

FIG. 4I shows the quantification of the cytotoxicity of PB NK cells against the indicated tumor cells at ratios of 0:1, 3:1, 5:1, and 10:1.

FIG. 5A is a schematic of an all-in-one, Tet-on 3G inducible system construct.

FIG. 5B are fluorescent images showing the dynamics of eGFP expression with and without dox treatment. Scale bars, 100 μm.

FIG. 5C shows the quantification of normalized mean fluorescence for eGFP expression over time with and without dox treatment.

FIG. 6 shows representative karyotyping analysis of ID2-H9 cells, which was normal.

FIG. 7A is a schematic of dox treatment for analysis of time-dependent ID2 expression on ID2-hPSCs.

FIG. 7B shows representative flow cytometry analysis of ID2 at the indicated time points.

FIG. 7C shows the quantification of ID2 expression (%) overtime.

FIG. 8A shows representative flow cytometry analysis of CD45 and CD56 expression on wild-type hPSC-derived NK cells using OP9 stromal feeder cells.

FIG. 8B shows the quantification of CD45+CD56+ cells for ID2-induced hPSC-NK cells, wild-type hPSC-NK cells derived on feeder cells, and PB NK cells.

FIG. 9 shows the quantification of cell viability (%) for hPSC-derived NK cells incubated with wild-type H9 hPSCs, hPSC-derived mesoderm, hPSC-derived endoderm, and hPSC-derived ectoderm at an effect-to-target ratio of 10:1. The number of viable cells was quantified. Data are represented as mean±s.d. of five independent replicates.

FIG. 10 is a schematic of the synergistically enhanced anti-tumor effect of dual CAR hPSC-NK cells.

FIG. 11A is a schematic of various lentiviral CAR constructs.

FIG. 11B shows killing of MDA-MB-231 tumor cells by NK-92 cells at different ratios of effector-to-target in the absence of 10 nM anti-fluorescein isothiocyanate (FITC)-folate adapter. Dataset A is NK 92, B is CAR #1 NK 92, C is CAR #2 NK92, D is CAR #3 NK 92, E is CAR #4 NK 92, F is CAR #5 NK 92, G is CAR #6 NK 92, H is CAR #7 NK 92, and I is CAR #8 NK 92. Data are represented as mean±s.d. of five independent replicates, *p<0.05.

FIG. 11C shows killing of MDA-MB-231 tumor cells by NK-92 cells at different ratios of effector-to-target in the presence of 10 nM FITC-folate adapter. Dataset A is NK92, B is CAR #1 NK 92, C is CAR #2 NK 92, D is CAR #3 NK 92, E is CAR #4 NK 92, F is CAR #5 NK 92, G is CAR #6 NK 92, H is CAR #7 NK 92, and I is CAR #8 NK 92. Data are represented as mean±s.d. of five independent replicates, *p<0.05.

FIG. 11D shows enzyme-linked immunosorbent assay (ELISA) analysis of secreted cytokine IFNγ from various NK-92 cells upon MDA-MB-231 stimulation. Datasets A are No FITC-FA and datasets B are with FITC-FA. Data are represented as mean s.d. of five independent replicates, *p<0.05.

FIG. 11E shows ELISA analysis of secreted cytokine tumor necrosis factor α (TNFα) from various NK-92 cells upon MDA-MB-231 stimulation. Datasets A are No FITC-FA and datasets B are with FITC-FA. Data are represented as mean±s.d. of five independent replicates, *p<0.05.

FIG. 11F shows representative flow cytometry analysis of phosphorylated STAT3 (pSTAT3) and STAT5 (pSTAT5) in indicated NK-92 cells upon MDA-MB-231 stimulation.

FIG. 11G shows expansion of the indicated NK-92 cells seven days after co-culture with MDA-MB-231 cells was quantified. Data are represented as mean±s.d. of five independent replicates, *p<0.05.

FIG. 11H is a schematic of an in vitro MDA-MB-231 tumor rechallenge model and a cytotoxicity assay.

FIG. 11I shows killing of MDA-MB-231 tumor cells by indicated NK-92 cells performed in the presence of 10 nM FITC-folate adapter at different time points. Dataset A is NK92, B is CAR #1 NK 92, C is CAR #2 NK 92, D is CAR #3 NK 92, E is CAR #4 NK 92, F is CAR #5 NK 92, G is CAR #6 NK 92, H is CAR #7 NK 92, and I is CAR #8 NK 92. Data are represented as mean±s.d. of five independent replicates, *p<0.05.

FIG. 12A shows flow cytometry analysis of NK cells derived from different hPSCs. Plots show histograms of control (A) and indicated NK cell-specific antibody (B).

FIG. 12B shows representative images of immunological synapses at the interface between tumor and indicated hPSC-NK cells by F-actin staining.

FIG. 12C shows the quantification of immunological synapses (%).

FIG. 12D shows flow cytometry analysis of interferon-gamma (INFγ)/CD107a in different NK cells upon MDA-MB-231 cell stimulation.

FIG. 12E shows ELISA analysis of secreted cytokine TNFα from indicated NK cells in response to MDA-MB-231 cells.

FIG. 12F shows ELISA analysis of secreted cytokine INFγ from indicated NK cells in response to MDA-MB-231 cells.

FIG. 12G shows killing of MDA-MB-231 tumor cells by indicated hPSC-NK cells at different effector-to-target ratios in the presence of 10 nM FITC-folate adapter.

FIG. 12H shows the quantification of expression of phosphorylated STAT3 (pSTAT3) and STAT5 (pSTAT5) in the indicated hPSC-NK cells upon MDA-MB-231 stimulation.

FIG. 12I shows the quantification of expansion of the indicated hPSC-NK cells seven days after co-culture with MDA-MB-231 tumor cells.

FIG. 12J shows the killing of MDA-MB-231 tumor cells by the indicated hPSC-NK cells in the presence of 10 nM FITC-folate adapter at different time points. Data are represented as mean±s.d. of five independent replicates, *p<0.05.

FIG. 13A is a schematic of subcutaneous injection of MDA-MB-231 cells for in vivo tumor model construction and persistence analysis of various hPSC-derived NK cells.

FIG. 13B shows flow cytometry analysis of CD45+CD56+ hPSC-NK cells in host blood at different time points after intravenous injection of the indicated hPSC-NK cells or phosphate-buffered saline (PBS) control.

FIG. 13C shows the quantification of CD45+CD56+ hPSC-NK cells (%) (n=5).

FIG. 13D shows the body weight of all experimental mouse groups measured at the indicated time points.

FIG. 13E shows hematoxylin and eosin stain (H&E) images of major organs collected at the end of the treatment described in FIG. 13A.

FIG. 14A is a schematic of the intravenous injection of various hPSC-NK cells for an in vivo anti-tumor cytotoxicity study.

FIG. 14B shows the results of the in vivo anti-tumor cytotoxicity study in which 5×10⁵ MDA-MB-231 cells were subcutaneously implanted into the left back of NRG mice. After 7 days, the mice were intravenously treated with PBS or 1×10⁷ hPSC-NK cells.

FIG. 14C shows the time-dependent tumor burden of experimental mouse groups treated as indicated as compared to PBS control.

FIG. 14D shows the level of released human TNFα in peripheral blood collected from the indicated experimental mice as measured by ELISA (n=5).

FIG. 14E shows the level of released interleukin 6 (IL-6) in peripheral blood collected from the indicated experimental mice as measured by ELISA (n=5).

FIG. 15A is a schematic of the in vivo tumor rechallenge model, in which 1×10⁵ MDA-MB-231 cells were subcutaneously implanted into the right back of NSG mice at day 37.

FIG. 15B shows time-dependent second tumor burden volume for the indicated experimental mouse groups.

FIG. 15C shows time-dependent second tumor burden volume for the indicated experimental mouse groups.

FIG. 15D shows a Kaplan-Meier curve, which demonstrates survival of the indicated experimental mouse groups (n=5).

FIG. 16A shows flow cytometry analysis of PD-L1 and folate receptor alpha (FRα) expression on LNCaP and MDA-MB-231 cells.

FIG. 16B shows the molecular structure of FITC-folate small molecule.

FIG. 16C shows the binding affinity of FITC-folate on MDA-MB-231 cells.

FIG. 16D shows the binding affinity of FITC-folate on NK-92 cells.

FIG. 16E shows flow cytometry analysis of anti-PD-L1 and anti-FITC CAR expression on NK-92 cells.

FIG. 17A shows killing of LNCaP tumor cell by indicated NK-92 cells at different ratios of effector-to-target in the absence of FITC-folate adapter. Data are represented as mean±s.d. of five independent replicates, *p<0.05.

FIG. 17B shows killing of LNCaP tumor cell by indicated NK-92 cells at different ratios of effector-to-target in the presence of FITC-folate adapter. Data are represented as mean z s.d. of five independent replicates, *p<0.05.

FIG. 17C shows ELISA analysis of IFNγ secreted from various NK-92 cells in response to LNCaP tumor cells.

FIG. 17D shows ELISA analysis of TNFα secreted from various NK-92 cells in response to LNCaP tumor cells.

FIG. 18A shows quantification of expression of phosphorylated pSTAT3 in the indicated hPSC-NK cells upon MDA-MB-231 stimulation. Data are represented as mean±s.d. of five independent replicates, *p<0.05.

FIG. 18B shows quantification of expression of phosphorylated pSTAT5 in the indicated hPSC-NK cells upon MDA-MB-231 stimulation. Data are represented as mean±s.d. of five independent replicates, *p<0.05.

FIG. 19A is a schematic of an anti-FITC CAR construct and a targeted knock-in strategy at the AAVS1 safe harbor locus. The vertical arrow indicates the AAVS1 targeting sgRNA. Horizontal arrows labeled (B) and horizontal arrows labeled (A) indicate primers for assaying targeting efficiency and homozygosity, respectively.

FIG. 19B shows PCR genotyping of single cell-derived hPSC clones after puromycin selection, and the expected PCR product for correctly targeted AAVS1 site is 991 bp (arrow) with an efficiency of 7 clones from a total of 11 clones. Homozygosity assay of the knock-in clones showed that hose without ˜240 bp PCR products were homozygous (arrow).

FIG. 19C shows flow cytometry analysis of anti-PD-L1, anti-FITC CAR, OCT-4, and SSEA-4 expression in wild-type and CAR-engineered hPSCs.

FIG. 20A is a schematic of hematopoietic and NK cell differentiation from hPSCs.

FIG. 20B shows representative flow cytometry analysis of CD34 and CD45 expression on the indicated hPSC differentiation cultures at day 0 and 15.

FIG. 20C shows representative flow cytometry analysis of CD56 and CD45 expression on the indicated hPSC differentiation cultures at day 15 and 45.

FIG. 21A shows the number of immunological synapses formed between the indicated NK cells and LNCaP tumor cells.

FIG. 21B shows the flow cytometry analysis of INFγ/CD107a expression on the indicated hPSC-NK cells in response to LNCaP tumor cells.

FIG. 21C shows ELISA analysis of secreted TNFα from the indicated hPSC-NK cells in response to LNCaP tumor cells.

FIG. 21D shows ELISA analysis of secreted INFγ from the indicated hPSC-NK cells in response to LNCaP tumor cells.

FIG. 21E shows killing of LNCaP tumor cells by the indicated hPSC-NK cells at different ratios of effector-to-target in the presence of 10 nM FITC-folate adapter.

FIG. 22A shows representative flow cytometry analysis of pSTAT3 and pSTAT5 expression in the indicated hPSC-NK cells upon MDA-MB-231 stimulation.

FIG. 22B shows quantification of cell viability (%) for hPSC-NK cells incubated with normal H9 hPSCs, hPSC-derived mesoderm, hPSC-derived endoderm, and hPSC-derived ectoderm at an effector-to-target ratio of 10:1. Data are represented as mean±s.d. of five independent replicates.

FIG. 23A shows flow cytometry analysis of CD45+CD56+ hPSC-NK cells in host blood at different time points after intravenous injection of indicated hPSC-NK cells or PBS control.

FIG. 23B shows the quantification of CD45+CD56+ hPSC-NK cells (n=5).

FIG. 23C shows the body weight of all experimental mouse groups at the indicated time points.

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

The present disclosure is predicated, at least in part, on the use of a transcription factor (TF)-mediated cell fate engineering approach to develop a robust and rapid platform for scalable production of natural killer (NK) cells from human pluripotent stem cells (hPSCs).

“Natural killer” and “NK” are used to refer to a subset of peripheral blood lymphocytes defined by the expression of CD56 or CD16 and the absence of the T cell receptor (CD3). The majority of the NK cells can be CD45+CD56+. The NK cells can express at least one NK cell-specific marker. The at least one NK cell-specific marker can be NKp44, NKp46, KIR3DL1, NKG2D, or any combination thereof.

The use of “pluripotent” to describe stem cells refers to the ability of the cells to form all cell lineages of an organism—in this case, all cell lineages of a human. Pluripotency characteristics include, but are not limited to, morphology (e.g., small, round, high nucleus-to-cytoplasm ratio, notable presence of nucleoli, and inter-cell spacing), the potential for unlimited self-renewal, the expression of pluripotent stem cell markers (e.g., SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30, and/or CD50), the ability to differentiate into ectoderm, mesoderm, and endoderm, teratoma formation, and formation of embryoid bodies.

The hPSCs hereof are genetically modified (using, for example, the CRISPR/Cas9-mediated gene knock-in technique) to introduce an inducible inhibitor of DNA binding 2 (ID2) construct, nuclear factor interleukin 3-regulated (NFIL3) construct, and/or Spi-1 proto-oncogene (SPI1) construct into the adeno-associated virus site 1 (AAVS1) safe harbor locus. Heinze et al. (2019), supra. Accordingly, provided is a population of universal NK cells derived from hPSCs and engineered to overexpress the transcription factor ID2, NFIL3, and/or SPI1. As used herein, “universal” means that the population of NK cells can be administered to any human. Thus, the population of universal NK cells can be used as an “off-the-shelf” product in various therapies, such as targeted cancer immunotherapy. The resulting hPSC-derived NK cells can exhibit various mature NK-specific markers and display effective tumor-killing ability across various cancer cells in vitro. Thus, the present disclosure provides a new platform for efficient production of universal NK cells, which can be used in various therapies, such as targeted cancer immunotherapy, to treat any patient without human leukocyte antigen matching.

The present disclosure is further predicated on the use of chimeric antigen receptor (CAR) constructs to promote hPSC-NK cell proliferation and cytotoxicity against tumor cells, such as through antigen-dependent activation of phosphorylated STAT3 (pSTAT3) and phosphorylated STAT5 (pSTAT5) signaling pathways via an intracellular truncated IL-2 receptor β-chain (ΔIL-2Rβ) and STAT3-binding tyrosine-X-X-glutamine (YXXQ) motif. To develop a safer CAR-NK cell therapy that is universal and potent, certain embodiments of the NK cells hereof are equipped with a stable anti-fluorescein isothiocyanate (FITC)-CAR. When administered to a subject, the anti-FITC-folate adapter bridges programmable anti-FITC-CAR and folate receptor alpha (FRα)-expressing tumor cells, such as breast tumors, and can be used to boost further the anti-tumor activities of programmed death-ligand (PD-L1)-induced memory-like hPSC-NK cells. Thus, the present disclosure further provides NK cells with enhanced antigen-specific proliferation and anti-tumor toxicity, thereby providing universal NK cells with immunological memory-like phenotypes for targeted immunotherapy. Indeed, the hPSC-derived CAR-NK and CAR-NK-92 cells hereof demonstrated controllable and potent antitumor activities.

In the pursuit of achieving higher efficacy and potency in NK cells, the NK cells hereof can further be engineered to express a second anti-PD-L1 CAR to leverage the striking clinical efficacy shown by checkpoint inhibitors that target PD-1 or PD-L1. Targeting PD-L1 can allow selective targeting of solid tumor cells and side effect profiles would be predicted based on PD-1/L1 immune checkpoint blockade. Robbins et al., Tumor control via targeting pd-li with chimeric antigen receptor modified nk cells, eLife (2020).

Due to their innate immunity against all kinds of pathogens and the unique property of not causing graft versus host diseases in allogenic transplantation, adoptive CAR-NK cell therapy holds great promise in treating various cancers. To date, most efforts have been conducted to treat hematologic malignancies with FDA-approved anti-CD19 or anti-CD33 CAR NK cells. Liu et al. (2018), supra; Roex et al., Two for one: targeting BCMA and CD19 in B-cell malignancies with off-the-shelf dual-CAR NK-92 cells, J Translational Medicine 20: 124 (2022); Ingegnere et al., Human CAR NK cells: A new non-viral method allowing high efficient transfection and strong tumor cell killing, Frontiers in Immunology 10 (2019); Liu et al., Use of CAR-Transduced Natural Killer Cells in CD19-Positive Lymphoid Tumors, New England J Medicine 382: 545-553 (2020).

### hPSCs and NK Cells

Universal NK cells (or a population of universal NK cells) derived from hPSCs are provided. In certain embodiments, the population of universal NK cells is derived from hPSCs and engineered to overexpress the transcription factor ID2, NFIL3, and/or SPI1. The expression of the transcription factor(s) can be inducible.

The universal NK cells or population thereof can be differentiated from hPSCs using methods known in the art and/or exemplified herein. The hPSCs (e.g., a population of hPSCs) can comprise human embryonic stem cells (hESCs) and/or induced pluripotent stem cells (iPSCs). In one embodiment, the hPSCs can be autologous cells, although heterologous cells can also be used, such as when the patient being treated has received high-dose chemotherapy or radiation treatment to destroy the patient's immune system. In one embodiment, allogenic cells can be used. Where appropriate, the hPSCs can be obtained from a subject by means well-known in the art.

The hPSCs can be engineered to express an anti-PD-L1 CAR and an anti-FITC CAR. The hPSCs can be further engineered to overexpress transcription factor ID2, NFIL3, and/or SPI1. In certain embodiments, the hPSCs are engineered to express an anti-PD-L1 CAR and an anti-FITC CAR, and to overexpress at least transcription factor ID2.

The overexpression of one or more of transcription factors ID2, NFIL3, and/or SPI1 promotes NK cell generation under chemically defined, feeder-free culture conditions. “Feeder-free” refers to culture conditions essentially free of feeder or stromal cells and/or which has not been pre-conditioned by cultivation of feeder cells. “Pre-conditioned” refers to a medium harvested after feeder cells have been cultivated within the medium for a period of time, such as for at least one day. Pre-conditioned medium contains many mediator substances, including growth factors and cytokines secreted by the feeder cells cultivated in the medium.

The expression of the transcription factor can be controlled by operable linkage with a promoter. The selection of a promoter and its operable linkage with a sequence encoding a protein, such as ID2, is within the ordinary skill in the art. In various embodiments, the promoter is inducible.

In certain embodiments, the population of hPSCs and/or universal NK cells is engineered to overexpress the transcription factor ID2 (e.g., by engineering a population of hSPCs from which the universal NK cells are differentiated to overexpress the transcription factor ID2 or, alternatively, using a vector or other method). Desirably, the ID2 sequence is a human sequence; the sequence is conserved in chimpanzee, Rhesus monkey, cow, mouse, rat, chicken, zebrafish, and frog. The ID2 sequence is available on GenBank (Gene ID: 3398). The gene encoding ID2 is also known as BHLHb26; inhibitor of differentiation 2; GIG8; inhibitor of DNA binding 2, dominant negative helix-loop-helix protein; class B basic helix-loop-helix protein 26; DNA-binding protein inhibitor ID-2; cell growth-inhibiting gene 8; inhibitor of DNA binding 2, HLH protein; DNA-binding protein inhibitor ID2; helix-loop-helix protein ID2; BHLHB26; ID2A; and I1D2H. The protein encoded by the gene belongs to the inhibitor of DNA binding family, members of which are transcriptional regulators that contain a helix-loop-helix (HLH) domain but not a basic domain. Members of the family inhibit the functions of basis HLH transcription factors in a dominant-negative manner by suppressing their heterodimerization partners through the HLH domains. See, e.g., GeneCards®: The Human Gene Database, publicly available via the Internet.

In certain embodiments, the population of hPSCs and/or universal NK cells is engineered to overexpress the transcription factor NFIL3. Desirably, the NFIL3 sequence is a human sequence. The NFIL3 sequence is available on GenBank (Gene ID: 4783). The expression of the NFIL3 sequence can be controlled by operable linkage with a promoter. The selection of a promoter and its operable linkage with a sequence encoding a protein, such as NFIL3, is within the ordinary skill in the art. In various embodiments, the promoter is inducible.

The gene encoding NFIL3 is also known as E4BP4, NF-IL3A, NFIL3A, IL3BP1, interleukin-3 promoter transcriptional activator, nuclear factor interleukin-3-regulated protein, Adenovirus E4 promoter region binding protein, transcription activator NF-IL3A, interleukin-3-binding protein, E4 promoter-binding protein 4, interleukin-3 binding protein 1, E4 promoter-binding protein. The protein encoded by the gene is a transcriptional regulator that binds as a homodimer to activating transcription factor (ATF) sites in many cellular and viral promoters. The encoded protein represses PERT and PER2 expression and, therefore, plays a role in the regulation of circadian rhythm. See, e.g., GeneCards®: The Human Gene Database, publicly available via the Internet.

In certain embodiments, the population of hPSCs and/or universal NK cells is engineered to overexpress the transcription factor SPI1. Desirably, the SPI1 sequence is a human sequence. The SPI1 sequence is available on GenBank (Gene ID: 6688). The expression of the SPI1 sequence can be controlled by operable linkage with a promoter. The selection of a promoter and its operable linkage with a sequence encoding a protein, such as SPI1, is within the ordinary skill in the art. In various embodiments, the promoter is inducible.

The gene encoding SPI1 is also known as SPI-A, SFPI1, SPI-1, PU.1, OF, hematopoietic transcription factor PU.1, 31 kDa transforming protein, transcription factor PU.1, spleen focus forming virus (SFFV) proviral integration oncogene Spi1, SFFV proviral integration oncogene, 31 kDa-transforming protein, and AGM10. The gene encodes an ETS-domain transcription factor that activates gene expression during myeloid and B-lymphoid cell development. The nuclear protein binds to a purine-rich sequence known as the PU-box found near the promoters of target genes and regulates their expression in coordination with other transcription factors and cofactors. The protein can also regulate alternative splicing of target genes. See, e.g., GeneCards®: The Human Gene Database, publicly available via the Internet.

As noted above, CAR-expressing hPSCs are also provided, as are populations of universal NK cells derived therefrom. In certain embodiments, the population of universal NK cells expresses a PD-L1 CAR (i.e., the CAR-expressing hPSCs from which such population was derived comprises a PD-L1 CAR). In certain embodiments, the population of universal NK cells expresses an anti-FITC CAR (i.e., the CAR-expressing hPSCs from which such population was derived comprises a PD-L1 CAR). In certain embodiments, the population of universal NK cells expresses dual CAR constructs including a PD-L1 CAR and an anti-FITC CAR (i.e., the CAR-expressing hPSCs from which such population was derived comprises dual CAR constructs including a PD-L1 CAR and an anti-FITC CAR).

CARs are engineered receptors, which graft an arbitrary specificity onto an immune effector cell, such as an hPSC hereof or an hPSC-derived NK cell hereof. See, e.g., Sadelain et al., “The Basic Principles of Chimeric Antigen Receptor Design,” Cancer Discovery OF1-11 (2013). Non-limiting examples of complementarity-determining regions (CDRs) include, but are not limited to, CD19 (U.S. Pat. No. 7,446,190, and U.S. Patent Application No. 2013/0071414), HER2 (Ahmen et al., HER2-specific T cells target primary glioblastoma stem cells and induce regression of autologous experimental tumors, Clinical Cancer Research 16(2): 474-485 (2010)), MUC16 (Chekmasova et al., Successful eradication of established peritoneal ovarian tumors in SCID-Beige mice following adoptive transfer of T cells genetically targeted to the MUC16 antigen, Clinical Cancer Research 16(14): 3594-3606 (2011)), and prostate-specific membrane antigen (PSMA) (Zhong et al., Chimeric antigen receptors combining 4-1BB and CD28 signaling domains augment PI3kinase/AKT/Bcl-XL activation and CD8+ T cell-mediated tumor eradication, Molecular Therapy 18(2): 413-420 (2010)).

CAR-NK cells have been engineered from various NK cells, including NK-92 cell line, hPSC-derived, cord blood and peripheral blood NK cells, though NK-92 derived CAR-NK cells are dominant in clinical trials due to their excellent expansion capacity in vitro. While no obvious toxicity was observed in clinical trials with NK-92 cells, concerns still exist regarding their in vivo survival and proliferation after irradiation during cell preparation for infusion. Li et al. (2018), supra; Biederstadt & Rezvani, Engineering the next generation of CAR-NK immunotherapies, Int. J. Hematol. (2021).

Engineering CAR-NK cells from hPSCs allows for an unlimited cell source of universal “off-the-shelf” cellular products. In addition, the relative ease of genome editing in hPSCs also allows massive production of homogenous and stable CAR-expressing NK cells for a more standardized product on a clinical scale. Both CRISPR/Cas9-mediated knock-in and lentiviral transduction strategies can be used to introduce CAR constructs into hPSCs for functional CAR-NK cell production.

CARs can be a fusion protein comprising an extracellular domain, a transmembrane domain, and an intracellular domain. In certain embodiments, a CAR hereof binds a cell-surface antigen on an immunosuppressive cell or a cancerous cell with high specificity.

Additionally, “binds with specificity,” “binds with high specificity,” or “selectively” binds, when referring to a ligand/receptor, a recognition region/targeting moiety, a nucleic acid/complementary nucleic acid, an antibody/antigen, or other binding pair indicates a binding reaction that is determinative of the presence of the protein in a heterogeneous population of proteins and other biologics. Thus, under designated conditions, a specified ligand or recognition region binds to a particular receptor (e.g., one present on a cancer cell) or targeting moiety, respectively, and does not bind in a significant amount to other proteins present in the sample (e.g., those associated with normal, healthy cells). Specific binding or binding with high affinity can also mean, for example, that the binding compound, ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

In a typical embodiment, a molecule that specifically binds a target will have an affinity that is at least about 10⁶ liters/mol (K_{D}=10^{˜6} M), and preferably at least about 10 liters/mol, as determined, for example, by Scatchard analysis. It is recognized by one of skill in the art that some binding compounds can specifically bind to more than one target, for example an antibody specifically binds to its antigen, to lectins by way of the antibody's oligosaccharide, and/or to an Fc receptor by way of the antibody's Fc region.

The extracellular domain of a CAR can include an antigen binding/recognition region/domain. The antigen binding domain of the CAR can bind to a specific antigen, such as a cancer/tumor antigen (e.g., for the treatment of cancer), a pathogenic antigen, such as a viral antigen (e.g., for the treatment of a viral infection), or a CD antigen. Cancer/tumor antigens can be cell surface antigens of cancer cells including biomolecules that are specifically expressed, or whose expression level is increased (as compared to normal cells), in cancer cells and their progenitor cells. Examples of tumor antigens include, but are not limited to, carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, an antigen of a cytomegalovirus infected cell (e.g., a cell surface antigen), epithelial glycoprotein 2 (EGP2), epithelial glycoprotein 40 (EGP40), epithelial cell adhesion molecule (EpCAM), receptor tyrosine-protein kinase erb-B2, 3 or 4, folate-binding protein (FBP), FITC, fetal acetylcholine receptor (AChR), FRα, folate receptor β (FRβ), ganglioside G2 (GD2), ganglioside G3 (GD3), human epidermal growth factor receptor 2 (HER2), human telomerase reverse transcriptase (hTERT), interleukin 13 (IL-13) receptor subunit α2 (IL-13Rα2), κ light chain, kinase insert domain receptor (IDR), Lewis A (CA19.9), Lewis Y (LeY), L1 cell adhesion molecule (L1CAM), melanoma antigen family A1 (MAGE-A1), mucin 16 (Muc-16), mucin 1 (Muc-1), mesothelin (MSLN), NKG2D ligand, cancer-testis antigen NY-ESO-1, oncofetal antigen (h5T4), prostate stem cell antigen (PSCA), PSMA, tumor-associated glycoprotein 72 (TAG-72), vascular endothelial growth factor receptor (VEGF-R, such as R2), and Wilms tumor protein (Wt-1). In certain embodiments, the extracellular domain of the CAR comprises anti-FITC polypeptide.

In certain embodiments, the antigen binding/recognition region/domain of the CAR can be a scFv of an antibody, a Fab fragment or the like that binds to a cell-surface antigen (e.g., cluster of differentiation 19 (CD19)) with specificity (e.g., high specificity). Where the recognition region of the CAR comprises a scFv region, the scFv region can be prepared from (i) an antibody known in the art that binds a targeting moiety, and/or (ii) sequence variants derived from the scFv regions of such antibodies, e.g., scFv regions having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the scFv region from which they are derived.

“Percent (%) sequence identity” with respect to a reference to a polypeptide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence.

In certain embodiments, instead of a scFv, the antigen binding/recognition region/domain of the CAR can comprise a smaller anti-PD-L1 nanobody for tumor antigen targeting. Li et al. (2018), supra; Zhang et al., Structural basis of a novel PD-L1 nanobody for immune checkpoint blockade, Cell Discovery 3: 17004 (2017). There, the CAR can also further comprise a NK-specific 2B4 co-stimulatory domain.

Examples of transmembrane domains include, but are not limited to, a CD3ζ polypeptide, a CD4 polypeptide, a CD8 polypeptide, a CD28 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, and a BTLA polypeptide.

Clinical trials with anti-PD-L1 CAR-NK cells are currently underway (NCT04847466). Unlike previous CD28 transmembrane and FcεRIγ signaling domains, the CAR construct described herein can contain a NK-specific NKG2D transmembrane domain, and a CD3ζ signaling domain that is essential for both T and NK cell activation. Robbins et al. (2020), supra; Fabian et al., PD-L1 targeting high-affinity NK (t-haNK) cells induce direct antitumor effects and target suppressive MDSC populations, J ImmunoTherapy Cancer 8(1) (2020); Reighard et al., Therapeutic Targeting of Follicular T Cells with Chimeric Antigen Receptor-Expressing Natural Killer Cells, Cell Reports Medicine 1(1): 100003 (2020).

The intracellular domain can comprise, for example, a CD3ζ polypeptide, and can further comprise at least one costimulatory signaling region comprising at least one costimulatory molecule. “Costimulatory molecule” refers to a cell surface molecule, other than an antigen receptor/ligand required for an efficient response of lymphocytes to antigen. The costimulatory signaling region can comprise a CD28 (cluster of differentiation 28) polypeptide, a 4-1 BB polypeptide, a CD134 polypeptide (cluster of differentiation 134; OX40 polypeptide), a CD278 polypeptide (cluster of differentiation 278; an ICOS polypeptide), a DAP-10 polypeptide, a PD-1 polypeptide, a LAG-3 polypeptide, a 2B4 polypeptide, a BTLA polypeptide, or a CTLA-4 polypeptide. In certain embodiments, the intracellular domain comprises an NK-specific 2B4 co-stimulatory domain.

As noted above, the population of universal NK cells can be engineered to express dual CAR constructs, namely, a PD-L1 CAR and a FITC-CAR. PD-L1 is an inhibitory ligand that binds to D-1 to suppress T-cell activation. PD-L1 is constitutively expressed and induced in tumor cells. PD-L1 is also expressed in myeloid-derived suppressor cells (MDSCs) and tumor-associated macrophages (TAMs).

See International Patent Application Publication No. WO 2020/198128, which is incorporated by reference herein, for a discussion of engineered human NK cells with a switchable CAR and their use in the treatment of refractory cancers (hematological and solid tumors) and viral infections. As discussed therein, examples of switch targets for hematological malignancies include B-cell maturation antigen (BCMA), CD123, CD138, CD19, CD20, CD22, CD24, CD30, CD33, CD37, CD38, CD4, CD7, CD70, CLL1, CS1, κ light chain, and receptor tyrosine kinase-like orphan receptor (ROR1). Also as discussed therein, examples of switch targets for solid tumors include, but are not limited to, fetal acetylcholine receptor (AchR), B7-H4, carbonic anhydrase IX (CAIX), CD133, CD44v6, CD47, CD70, carcinoembryonic antigen (CEA), c-mesenchymal-epithelial transition factor (c-Met), delta-like 3 (DLL3), epidermal growth factor receptor (EGFR), EGFRvIII epithelial cell adhesion molecule (EpCAM), erythropoietin-producing hepatocellular carcinoma A2 (EphA2), ErbB2, fibroblast activation protein (FAP), FRα, Frizzled 7 (Fzd7), ganglioside GD2, glypican-3 (GPC3), guanylyl cyclase C (GUCY2C), human epidermal growth factor receptor 1 (HER1), HER2, intercellular adhesion molecule 1 (ICAM-1), interleukin 11 receptor α (IL-11Rα), interleukin 13 receptor α (IL-13Rα2), human L1 cell adhesion molecule (L1-CAM), Lewis Y antigen (LeY), melanoma-associated antigen (MAGE), melanoma cell adhesion molecule (MCAM), mesothelin, mucin 1 (MUC1), mucin 16 (MUC16 ecto), natural killer group 2 member D ligands (NKG2DLs), cancer/testis antigen 1 (NY-ESO-1), PD-L1, prostate stem cell antigen (PSCA), PSMA, receptor-tyrosine kinase-like orphan receptor (ROR1), tumor-associated glycoprotein 72 (TAG72), and vascular endothelial growth factor receptor 1 (VEGF R1). Further discussed therein are switch targets for viral infections, examples of which include, but are not limited to, HIV glycoprotein 120 (gp120), CD4, HBV surface antigen (HBsAg), EBV latent membrane protein 1 (LMP1), CMV glycoprotein B (gB), and HCV glycoprotein E2.

The CAR construct(s) hereof can be used to promote hPSC-NK cell proliferation and cytotoxicity against tumor cells, such as through antigen-dependent activation of phosphorylated STAT3 (pSTAT3) and phosphorylated STAT5 (pSTAT5) signaling pathways via an intracellular truncated IL-2 receptor β-chain (ΔIL-2Rβ) and STAT3-binding tyrosine-X-X-glutamine (YXXQ) motif. Accordingly, an anti-PD-L1 CAR and/or anti-FITC CAR can comprise a truncated cytoplasmic domain from interleukin-2 (IL-2) receptor β-chain (ΔIL-2Rβ), a STAT3-binding tyrosine-X-X-glutamine (YXXQ) motif, or both. See, e.g., U.S. Pat. No. 11,077,143 (incorporated by reference herein) for NK cells that express PD-L1 CAR, CD16 and IL-2 and their use to reduce tumor cells and other cells, such as MDSCs and TAMs, in the tumor microenvironment (see, also, U.S. Patent Application No. 2016/0009813 and U.S. Pat. No. 11,141,434, both of which are incorporated by reference herein). In certain embodiments, the hPSCs and/or the NK cells expressing such CAR constructs comprise a truncated cytoplasmic domain from IL-2 receptor β-chain, a STAT3-binding tyrosine-X-X-glutamine (YXXQ) motif, or both.

Inclusion of the truncated cytoplasmic domain from interleukin-2 (IL-2) receptor β-chain can, for example, facilitate antigen-inducible NK cell expansion. Since IL-2 plays a critical role in memory cell formation, reverses NK cell exhaustion, and promotes expansion of memory-like NK cells, inclusion of both the truncated cytoplasmic domain from interleukin-2 (IL-2) receptor β-chain and the a STAT3 signaling activation motif YXXQ (an IL-21-associated motif within the I1-21 receptor) in the anti-PD-L1 CAR construct can be advantageous and synergistic. Seo et al. (2017), supra; Kagoya et al. (2018), supra; Kasaian et al., IL-21 limits NK cell responses and promotes antigen-specific T cell activation: A mediator of the transition from innate to adaptive immunity, Immunity 16(4): 559-569 (2002); Venkatasubramanian et al., IL-21-dependent expansion of memory-like NK cells enhances protective immune responses against Mycobacterium tuberculosis, Mucosal Immunology 10(4): 1031-1042 (2017); Granzin et al., Highly efficient IL-21 and feeder cell-driven ex vivo expansion of human NK cells with therapeutic activity in a xenograft mouse model of melanoma, Oncoimmunology 5(9): e1219007 (2016).

The anti-PD-L1 CAR and/or anti-FITC CAR can also comprise NK cell-Fc receptor transmembrane and intracellular signaling domains, such as γ-chain from CD32a (or FcγRIIA) or CD16 (or FCγRIII).

Such modifications lead to robust activation of the STAT3 and STAT5 signaling pathway in a tumor antigen-responsive manner, and to CAR-NK cells with enhanced in vivo persistence and antitumor functions than that seen with single anti-FITC CAR NK cells lacking anti-PD-L1 CAR. Cell expansion was not observed in CAR-NK cells with anti-PD-L1 CAR co-culturing with PD-L1 rare tumor cells, further demonstrating the antigen specificity of the CAR-NK cell persistence for a safer and more durable antitumor immunity. The dual anti-FITC and anti-PD-L1 hPSC CAR-NK cells demonstrated improved universality, safety, potency, and persistence, both in vitro and in vivo, in an antigen-dependent manner, and achieved a memory-like phenotype of NK cells.

While the use of a CRISPR/Cas9-mediated gene knock-in technique is exemplified herein to introduce an inducible ID2 construct into the AAVS1 safe harbor locus to modify genetically hPSCs, any suitable method can be used to prepare the CAR constructs hereof and deliver a CAR-encoding nucleic acid, such as a plasmid, into hPSCs. Genome editing, also referred to as genomic editing or genetic editing, is a type of genetic engineering in which DNA is inserted, deleted and/or replaced in the genome of a targeted cell. Targeted editing can be achieved through a nuclease-independent or nuclease-dependent method. Nuclease-independent editing can involve homologous recombination guided by homologous sequences flanking an exogenous polynucleotide to be inserted into a genome. Alternatively, specific endonucleases can be used to introduce double-stranded breaks into the DNA, which then undergo repair. CRISPR/Cas9 (clustered regular interspaced short palindromic repeats associated 9) is an RNA-guided nuclease. Other endonucleases include, but are not limited to, zinc-finger nucleases (ZFN) and transcription activator-like effector nucleases (TALEN). Another system is DICE (dual integrase cassette exchange), which utilizes phiC31 and Bxb1 integrases for targeted integration. Other examples of genome editing methods include, but are not limited to, nucleofection/electroporation, transfection via Lipofectamine Stem (ThermoFisher, STEM00001) or similar transfection reagents, or lentivirus, retrovirus, sleeping beauty, piggyback (transposon/transposase systems including a non-viral mediated CAR gene delivery system) or adeno-associated virus (AAV)-mediated delivery.

While the AAVS1 safe harbor locus is exemplified herein, other sites for targeted integration include, but are not limited to, other safe harbor loci or genomic safe harbor (GSH), which are intragenic/extragenic regions of the human genome that, theoretically, are able to accommodate predictable expression of newly integrated DNA without adverse effects on the host cell or recipient organism. A useful safe harbor must permit sufficient transgene expression to yield desired levels of the vector-encoded protein or non-coding RNA. A safe harbor also must not predispose cells to malignant transformation or alter cellular functions. Ideally, the safe harbor locus is characterized by the absence of disruption of regulatory elements or genes, is an intergenic region in a gene dense area or a location at the convergence between two genes transcribed in opposite directions, keep distance to minimize the possibility of long-range interactions between vector-encoded transcriptional activators and the promoters of adjacent genes (in particular cancer-related and microRNA genes), and has ubiquitous transcriptional activity. The location should also be devoid of repetitive elements and conserved sequences and allow for easy design of primers for amplification. Suitable sites for human genome editing include, in addition to AAVS1, the chemokine (CC motif) receptor 5 gene locus, human orthologue of the mouse ROSA26 locus, the human orthologue of the mouse H11 locus, collagen loci, and HTRP loci. The selected site must be validated for specific integration and, oftentimes, the insertion strategy, promoter, gene sequence, and construct design require optimization.

In certain embodiments, the CAR construct comprises one or more sequences that encode: an anti-FITC polypeptide or an anti-PD-L1 polypeptide; a NKG2d transmembrane domain; and a 2B4 co-stimulatory domain. The CAR construct can further comprise one or more sequences that encode a truncated cytoplasmic domain from IL-2 receptor β-chain, a STAT3 binding tyrosine-X-X-glutamine (YXXQ) motif, or both. The construct can further comprise one or more sequences that encode FcγRIII.

Constructs encoding the CARs can be prepared using genetic engineering techniques. Some of such techniques are described in detail in Sambrook et al., “Molecular Cloning: A Laboratory Manual,” 3rd Edition, Cold Spring Harbor Laboratory Press, (2001), and Green and Sambrook, “Molecular Cloning: A Laboratory Manual,” 4th Edition, Cold Spring Harbor Laboratory Press, (2012), which are both incorporated herein by reference in their entireties.

By way of non-limiting examples, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein comprising a recognition region, one or more co-stimulation domains, and an activation signaling domain, in frame and linked in a 5′ to 3′ direction. Other arrangements are also acceptable and include a recognition region, an activation signaling domain, and one or more co-stimulation domains.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. Nucleic acid vectors can have specialized functions such as expression, packaging, pseudotyping, or transduction. Vectors can also have manipulatory functions if adapted for use as a cloning or shuttle vector. The term “vector” as used herein comprises the construct to be delivered. The structure of the vector can include any desired form that is feasible to make and desirable for a particular use. Such for can include, for example, circular forms such as plasmids and phagemids, as well as linear or branched forms. A nucleic acid vector can be composed of, or example, DNA or RNA, as well as contain partially or fully, nucleotide derivatives, analogs or mimetics. Such vectors can be obtained from natural sources, produced recombinantly or chemically synthesized. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

The placement of the antigen binding/recognition region/domain in the fusion protein will generally be such that display of the region on the exterior of the cell is achieved. Where desired, the CARs can also include additional elements, such as a signal peptide (e.g., CD8α signal peptide) to ensure proper export of the fusion protein to the cell surface, a transmembrane domain to ensure the fusion protein is maintained as an integral membrane protein (e.g., CD8α transmembrane domain, CD28 transmembrane domain, or CD3ζ transmembrane domain), and a hinge domain (e.g., CD8α hinge) that imparts flexibility to the recognition region and allows strong binding to the targeting moiety.

NK cells can be genetically engineered to express CAR constructs through targeted integration and/or using methods known in the art and/or exemplified herein. “Targeted integration” refers to a process involving insertion of one or more exogenous sequences, with or without deletion of an endogenous sequence at the insertion site. Targeted insertion can be achieved either through a nuclease-independent approach or through a nuclease-dependent approach. In the nuclease-independent targeted integration approach, homologous recombination can be guided by homologous sequences flanking an exogenous polynucleotide to be inserted through the enzymatic machinery of the host cell. For example, nuclease-independent targeted integration can comprise transfecting a population of hPSCs with an expression vector encoding the CAR construct. Suitable methods for preparing a transduced population of lymphocytes expressing a selected CAR construct are well-known to the skilled artisan. While the use of a CRISPR/Cas9-mediated gene knock-in technique is exemplified herein to introduce a construct into the AAVS1 safe harbor locus to modify genetically hPSCs, any suitable genome editing method can be used.

Alternatively, NK cells can be genetically engineered to express CAR constructs with by introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ can lead to random insertions or deletions of a small number of endogenous nucleotides. However, when a donor vector containing exogenous genetic material flanked by a pair of homology arms is present, the exogenous genetic material can be introduced into the genome during homology directed repair by homologous recombination, resulting in the targeted integration.

Available endonucleases capable of introducing specific and targeted DSBs include, without limitation, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases can also by used for targeted integration. The CRISPR/Cas9 system in particular is now widely used to induce targeted genetic alterations (genome alterations). Other non-limiting examples of targeted nucleases include naturally occurring and recombinant nucleases, e.g. CRISPR/Cas9, restriction endonucleases, meganucleases horning endonucleases, and the like.

CRISPR/Cas9 requires two major components: (1) a Caspase-9 endonuclease (Casp9) and (2) the crRNA-tracrRNA complex. When co-expressed, the two components form a complex that is recruited to a target DNA sequence comprising a protospacer flanking motif (PAM) sequence and a seeding region near PAM. The crRNA and tracrRNA can be combined to form a chimeric guide RNA (gRNA) to guide Casp9 to target selected sequences. These two components can then be delivered to mammalian cells via transfection or transduction. Cas proteins other than Cas9 can also be used, including, without limitation Cas12a or CasX.

In one embodiment, the cells used in the methods described herein can be autologous cells, although heterologous cells can also be used, such as when the patient being treated has received high-dose chemotherapy or radiation treatment to destroy the patient's immune system. In one embodiment, allogenic cells can be used.

Generally, after the cells to be engineered are obtained, the cells are cultured. Unlike T cells, prior sensitization and antigen exposure are not required for NK cells and, as such, the cells need not be cultured under conditions that promote the activation of the cells, but can be if desired.

In at least one embodiment, the culture conditions are such that the cells can be administered to a subject without concern for reactivity against components of the culture medium. For example, culture media that lacks any animal products, such as bovine serum albumin, can be used to culture engineered cells. In another embodiment, tissue culture conditions typically used by the skilled artisan to avoid contamination with bacteria, fungi and mycoplasma can be used. In one aspect, the activation can be achieved by introducing known activators into the culture medium, such as anti-CD3 antibodies in the case of cytotoxic T cells. Other suitable activators are generally known and include, for example, anti-CD28 antibodies. The population of cells can be cultured under conditions promoting activation for about 1 to about 4 days, for example. The appropriate level of activation can be determined by cell type, size, proliferation rate, or activation markers determined by flow cytometry.

In at least one embodiment, after the population of cells has been cultured, the cells are transfected with an expression vector encoding a CAR. Suitable vectors and transfection methods for use in various embodiments are known in the art. After transfection, the cells can be immediately administered to the patient or the cells can be cultured for a time period to allow time for the cells to recover from the transfection, for example, at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days. In one aspect, suitable culture conditions can be similar to the conditions under which the cells were cultured, either with or without an agent for promoting activation.

Thus, as described above, the methods of use and/or treatment described herein can further comprise 1) obtaining a population of autologous or heterologous NK cells, 2) culturing the cells, and 3) engineering the cells to express one or more CAR constructs through targeted integration. In certain embodiments, the methods of use and/or treatment described herein can comprise A) obtaining a population of autologous or heterologous hPSCs; B) engineering the cells to express one or more CAR constructs through targeted integration; and C) differentiating the engineered hPSCs into the universal NK cells or CAR-NK cells hereof.

### Compositions

Even still further provided is a pharmaceutical composition. The pharmaceutical composition can comprise a population of isolated universal NK cells and/or CAR-NK cells described herein or otherwise obtained in accordance with a method hereof. The term “isolated” means that the material is removed from its original environment, e.g., the natural environment if it is naturally occurring. For example, a naturally occurring neutrophil present within a living organism is not isolated, but the same neutrophil separated from some or all the coexisting materials in the natural system is isolated.

The pharmaceutical composition can further comprise one or more pharmaceutically acceptable carriers, diluents, and/or other pharmaceutically acceptable components. The term “pharmaceutically acceptable” and grammatical variations thereof, as they refer to compositions, carriers, diluents, reagents, and the like, are used interchangeably, are art-recognized, and indicate that the materials can be administered to or upon a mammal without undue toxicity, irritation, allergic response, and/or the production of undesirable physiological effects, such as nausea, dizziness, gastric upset, and the like as is commensurate with a reasonable benefit/risk ratio. In other words, it is a material that is not biologically or otherwise undesirable—i.e., the material can be administered to an individual along with NK cells, for example, without causing any undesirable biological effects or interacting in a significantly deleterious manner with any of the other components of the pharmaceutical composition.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting a composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethanol; (20) phosphate buffered solutions; and (21) other non-toxic, compatible substances employed in pharmaceutical formulations.

The choice of carrier will be determined in part by the particular CAR, CAR-encoding nucleic acid sequence, vector, or host cells expressing the CAR, as well as by the particular method used to administer the CAR-encoding nucleic acid sequence, vector, or host cells expressing the CAR. Accordingly, there are a variety of suitable formulations of the pharmaceutical composition. For example, the pharmaceutical composition can contain preservatives. Suitable preservatives may include, for example, methylparaben, propylparaben, sodium benzoate, and benzalkonium chloride. A mixture of two or more preservatives optionally can be used. The preservative or mixtures thereof are typically present in an amount of about 0.0001% to about 2% by weight of the total composition.

The carriers, diluents, and/or other components can be determined in part by the particular route of administration (see, e.g., Remington's Pharmaceutical Sciences, 17^{th} ed. (1985)). For example, a formulation suitable for systemic, e.g., intravenous, administration, may differ from a formulation suitable for intracranial administration. In certain embodiments, the ingredients of the composition are of sufficiently high purity and sufficiently low toxicity such that the composition is suitable for administration to a human. The composition desirably is stable. Such modifications are within the ordinary skill in the art.

Exemplary compositions comprising engineered CAR-NK cells include compositions comprising the cells in sterile 290 mOsm saline, in infusible cryomedia (containing Plasma-Lyte A, dextrose, sodium chloride injection, human serum albumin and DMSO), in 0.9% NaCl with 2% human serum albumin, or in any other sterile 290 mOsm infusible materials. In certain embodiments, prior to being administered to a patient, the cells are pelleted, washed, and are resuspended in a pharmaceutically acceptable carrier or diluent.

### Methods and Uses

Uses of any of the CAR-expressing NK cells provided herein, any universal NK cells described herein, any of the engineered hPSCs described herein, any of the constructs described herein, or pharmaceutical compositions hereof in the treatment of a disease, and/or in the manufacture of a medicament for the treatment of a disease in a subject are provided. In certain embodiments, the disease is cancer. “Cancer” includes any neoplastic condition, whether malignant, pre-malignant or non-malignant. Generally, however, the neoplastic condition is malignant. Both solid and non-solid tumors are encompassed, and “cancer(ous) cell” may be used interchangeably with “tumor(ous) cell.”

Examples of cancers include, but are not limited to, leukemia (e.g., ALL, AML, CLL, and CML), adrenocortical carcinoma, AIDS-related cancer (e.g., Kaposi sarcoma), lymphoma (e.g., T-cell, Hodgkins, and non-Hodgkins), astrocytoma, basal cell carcinoma, bladder cancer, bone cancer, brain cancer, breast cancer, prostate cancer, lung cancer, cervical cancer, colon cancer, colorectal cancer, DCIS, esophageal cancer, gastric cancer, glioma, head and neck cancer, liver cancer, stomach cancer, pancreatic cancer, kidney cancer (e.g., renal cell and Wilms), oral cancer, oropharyngeal cancer, ovarian cancer, testicular cancer, and throat cancer.

A method of producing a population of NK cells is provided. In certain embodiments, the method comprises differentiating a population of hPSCs to NK cells, wherein the population of hPSCs are engineered to overexpress transcription factor ID2, NFIL3, and/or SPI1. In certain embodiments, the population of hPSCs is engineered to overexpress transcription factor ID2. The population of hPSCs can be engineered to express an anti-PD-L1 CAR and an anti-FITC CAR. The hPSCs can comprise hESCs and/or iPSCs. The overexpression of the transcription factor(s) can be inducible.

The anti-PD-L1 CAR and/or anti-FITC CAR can comprise a truncated cytoplasmic domain from IL-2 receptor β-chain, a STAT3-binding tyrosine-X-X-glutamine (YXXQ) motif, or both. The anti-PD-L1 CAR and/or the anti-FITC CAR can comprise NK cell-Fc receptor transmembrane and intracellular signaling domains. The NK cell-Fc receptor transmembrane and intracellular signaling domains can comprise a γ-chain from CD32a or a γ-chain from CD16.

A method of treating cancer in a subject (e.g., in need thereof) is also provided. The method can comprise administering to the subject a first therapy comprising a therapeutically effective amount of a population of any of the universal NK cells described herein, a population any of the NK cells expressing one or more constructs described herein, a population of any of the CAR-expressing NK cells described herein, and/or a pharmaceutical composition described herein, and a pharmaceutically acceptable carrier and/or diluent.

The method of treating cancer in a subject can further comprise administering to the subject a conjugate (e.g., a therapeutically effective amount of a conjugate). The conjugate can comprise FITC linked to a ligand that binds FRα. In certain embodiments, the conjugate comprising FITC linked to a ligand that binds FRα has the following structure:

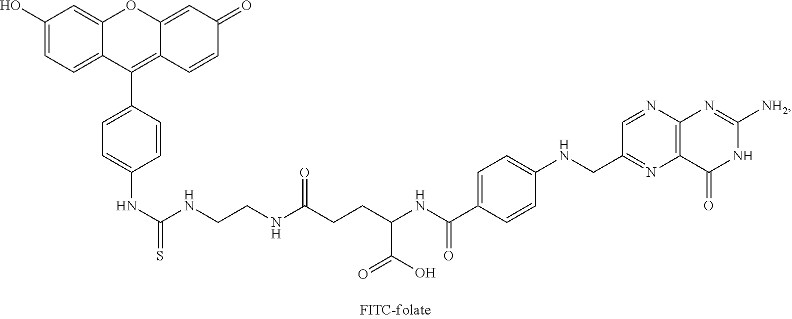 [SMILES: Nc1nc2ncc(CNc3ccc(C(=O)NC(CCC(=O)NCCNC(=S)Nc4ccc(-c5c6ccc(=O)cc-6oc6cc(O)ccc56)cc4)C(=O)O)cc3)nc2c(=O)[nH]1]

or is a pharmaceutically acceptable salt thereof.

The conjugate can comprise FITC linked to a ligand that binds PSMA. In certain embodiments, the ligand that binds PSMA is DUPA. In certain embodiments, the conjugate comprising FITC linked to a ligand that binds PSMA has the following structure:

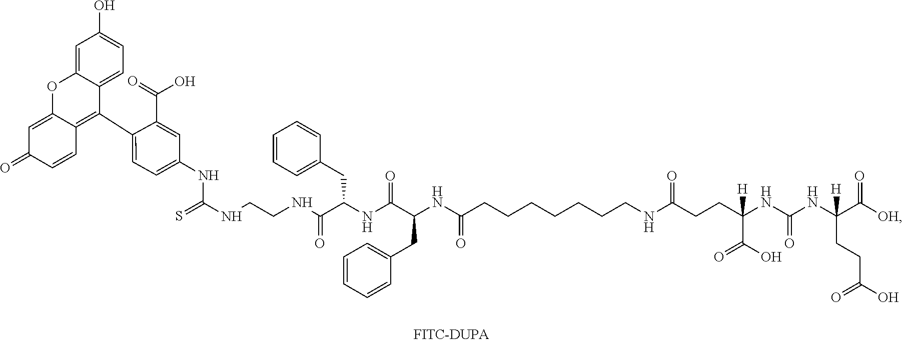 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

or is a pharmaceutically acceptable salt thereof.

The conjugate can comprise FITC linked to a ligand that binds carbonic anhydrase IX (CAIX). In certain embodiments, the conjugate has the following structure.

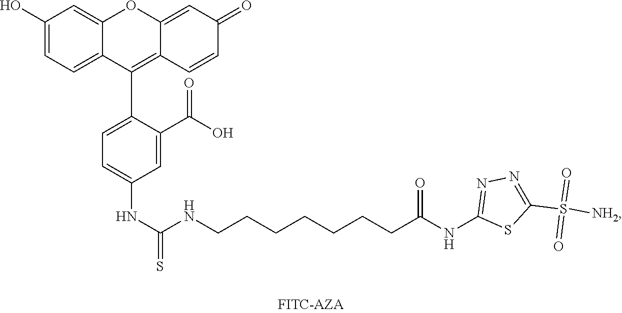 [SMILES: NS(=O)(=O)c1nnc(NC(=O)CCCCCCCNC(=S)Nc2ccc(-c3c4ccc(=O)cc-4oc4cc(O)ccc34)c(C(=O)O)c2)s1]

or is a pharmaceutically acceptable salt thereof. Such conjugates can be synthesized in accordance with methods known in the art.

The method can further comprise administering to the subject a second therapy. The second therapy can comprise surgical removal of one or more cancerous cells from the subject, chemotherapy, and/or radiotherapy (e.g., a therapeutically effective amount thereof). In certain embodiments, the method further comprises administering to the subject a therapeutically effective amount of chemotherapy to the subject. In certain embodiments, the method further comprises administering to the subject a therapeutically effective amount of radiotherapy to the subject. In certain embodiments, the method further comprises administering to the subject a therapeutically effective amount of both chemotherapy and radiotherapy to the subject.

The second therapy can alternatively or further comprise surgical removal of cancerous cells from the subject.

The second therapy can additionally or alternatively comprise imaging a targeted location (e.g., a cancer (e.g., a tumor microenvironment)) in the subject prior to or during administering the first and/or second therapies.

In some embodiments, the targeted location is additionally imaged prior to administration to the subject of the universal NK cells, the CAR-NK cells, or the NK cell composition. The cancer can be imaged during or after administration to assess metastasis, for example, and the efficacy of treatment. In some embodiments, imaging occurs by positron emission tomography (PET) imaging, magnetic resonance imaging (MRI), or single-photon-emission computed tomography (SPECT)/computed tomography (CT) imaging. The imaging method can be any suitable imaging method known in the art.

In certain embodiments, the first and second therapies are administered sequentially and/or alternatively relative to each other. In some embodiments, the method further comprises imaging the cancer in the subject prior to or during administering of the universal NK cells, the CAR-NK cells, the composition comprising the NK cells, and/or the second therapy.

The terms “treat,” “treating,” “treated,” and “treatment” (with respect to a disease or condition, such as cancer) are used to describe a method for obtaining beneficial or desired results, such as clinical results, which can include, but are not limited to, one or more of improving a condition associated with a disease, curing a disease, lessening severity of a disease, increasing the quality of life of one suffering from a disease, prolonging survival and/or a prophylactic treatment. In reference to cancer, in particular, the terms “treat,” “treating,” “treated,” or “treatment” can additionally mean reducing the size of a tumor, completely or partially removing the tumor (e.g., a complete or partial response), stabilizing a disease, preventing progression of the cancer (e.g., progression-free survival), or any other effect on the cancer that would be considered by a physician to be a therapeutic or prophylactic treatment of the cancer. More particularly, curative treatment refers to any of the alleviation, amelioration and/or elimination, reduction and/or stabilization (e.g., failure to progress to more advanced stages) of a sign/symptom, as well as delay in progression of a sign/symptom of a particular disorder. Prophylactic treatment refers to any of the following: halting the onset, reducing the risk of development, reducing the incidence, delaying the onset, reducing the development, and increasing the time to onset of symptoms of a particular disorder. Desirable effects of treatment can include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions are used to delay development of a disease and/or tumor, or to slow (or even halt) the progression of a disease and/or tumor growth.

The term “patient” or “subject” includes human and non-human animals, such as companion animals (dogs and cats and the like) and livestock animals. Livestock animals are animals raised for food production. The subject to be treated is preferably a mammal, in particular a human being.

In various embodiments, the universal NK cells and/or CAR-NK cells hereof (collectively referred to herein as the “NK cells hereof”) and pharmaceutical compositions hereof can be administered to the subject via any suitable route, such as parenteral administration, e.g., intradermally, subcutaneously, intramuscularly, intraperitoneally, intravenously, or intrathecally. As used herein, the term “administering” includes all means of introducing the NK cells hereof or pharmaceutical compositions comprising same to the patient. Examples include, but are not limited to, oral (po), parenteral, systemic/intravenous (iv), intramuscular (im), subcutaneous (sc), transdermal, intrasternal, intraarterial, intraperitoneal, epidural, intraurethral, intranasal, buccal, ocular, sublingual, vaginal, rectal, and the like. Routes of administration to the brain include, but are not limited to, intraparenchymal, intraventricular, intracranial, and the like.

The formulation of compositions suitable for administration of NK cells hereof, including compositions suitable for administration by intravenous and intratumoral routes, is within the ordinary skill in the art.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients, such as salts, carbohydrates and buffering agents (preferably at a pH in the range from about 3 to about 9). The preparation of parenteral formulations under sterile conditions may readily be accomplished using standard pharmaceutical techniques well-known to those skilled in the art.

The NK cells hereof can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrastemal, intracranial, intratumoral, intramuscular, topical, inhalation and/or subcutaneous routes. Indeed, the NK cells hereof, or composition comprising the NK cells hereof, can be administered directly into the blood stream, into muscle, or into an internal organ.

The NK cells hereof and related compositions can be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9).

The percentage of the NK cells hereof in the compositions and preparations can vary and can be between about 1 to about 99% weight of the active ingredient(s) and a binder, excipients, a disintegrating agent, a lubricant, and/or a sweetening agent (as are known in the art). The amount of the NK cells hereof in such therapeutically useful compositions is such that an effective dosage level will be obtained. The total number of NK cells hereof, and the concentration of the cells, in the composition administered to the patient can vary depending on a number of factors including, without limitation, the binding specificity of the CAR (where applicable), the identity of the cancer, the location of the cancer in the patient, the means used to administer the compositions to the patient, and the health, age and weight of the patient being treated. In various embodiments, suitable compositions comprising engineered cells include those having a volume of about 0.1 ml to about 200 ml and about 0.1 ml to about 125 ml.

The term “therapeutically effective amount” as used herein, refers to that amount of the NK cells hereof that elicits the biological or medicinal response in a tissue system, animal or human that is being sought by a researcher, veterinarian, medical doctor or other clinician (e.g., a desired therapeutic effect), which includes alleviation of the symptoms of the cancer being treated. In one aspect, the therapeutically effective amount is that which can treat or alleviate the cancer or symptoms thereof at a reasonable benefit/risk ratio applicable to any medical treatment. However, it is to be understood that the total daily usage of the NK cells hereof can be decided by the attending physician within the scope of sound medical judgment. In the treatment of cancer, a desired therapeutic effect can range from inhibiting the progression of cancer, e.g., proliferation of cancerous cells and/or the metastasis thereof. Desirably, the administration of a therapeutically effective amount kills cancerous cells, such that the number of cancerous cells decreases, desirably to the point of eradication.

The exact amount of the NK cells hereof required will vary from one subject to the next, depending on factors including the type of cancer being treated and the state/severity of the cancer; the specific composition employed; the age, body weight, general health, gender and diet of the patient; the time and route of administration; the duration of the treatment; drugs and/or other therapies used in combination or coincidentally with the NK cells hereof; and like factors well-known to the researcher, veterinarian, medical doctor or other clinician of ordinary skill. By way of example, a dose of the NK cells hereof can range from 10⁵ to 10¹² per m² of the patient's body surface area or per kg of the patient's weight. In certain embodiments, the therapeutically sufficient amount is at or about 10⁷ cells/kg of the patient's weight (such as, 10⁷ cells/kg). Thus, the absolute amount of the NK cells hereof included in a given unit dosage form can vary widely, and depends upon factors such as the age, weight and physical condition of the subject, as well as the method of administration.

Depending upon the route of administration, a wide range of permissible dosages are contemplated herein. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

Multiple infusions may be required to treat a subject effectively. For example, 2, 3, 4, 5, 6 or more separate infusions may be administered to a patient at intervals of from about 24 hours to about 48 hours, or every 3, 4, 5, 6, or 7 days. Infusions may be administered weekly, biweekly, or monthly. Monthly administrations can be repeated from 2-6 months or longer, such as 9 months to year.

Administered dosages for the NK cells hereof for treating cancer are in accordance with dosages and scheduling regimens practiced by those of skill in the art. Typically, doses >10⁹ cells/patient are administered to patients receiving adoptive cell transfer therapy. Determining an effective amount or dose is well within the capability of those skilled in the art, especially in light of the detailed disclosure provided herein.

The NK cells hereof administered to a subject can comprise about 1×10⁵ to about 1×10¹⁵ or 1×10⁶ to about 1×10¹⁵ transduced cells, for example. In various embodiments about 1×10⁵ to about 1×10¹⁰, about 1×10⁶ to about 1×10¹⁰, about 1×10⁶ to about 1×10⁹, about 1×10⁶ to about 1×10⁸, about 1×10⁶ to about 2×10⁷, about 1×10⁶ to about 3×10⁷, about 1×10⁶ to about 1.5×10⁷, about 1×10⁶ to about 1×10⁷, about 1×10⁶ to about 9×10⁶, about 1×10⁶ to about 8×10⁶, about 1×10⁶ to about 7×10⁶, about 1×10⁶ to about 6×10⁶, about 1×10⁶ to about 5×10⁶, about 1×10⁶ to about 4×10⁶, about 1×10⁶ to about 3×10⁶, about 1×10⁶ to about 2×10⁶, about 2×10⁶ to about 6×10⁶, about 2×10⁶ to about 5×10⁶, about 3×10⁶ to about 6×10⁶, about 4×10⁶ to about 6×10⁶, about 4×10⁶ to about 1×10⁷, about 1×10⁶ to about 1×10⁷, about 1×10⁶ to about 1.5×10⁷, about 1×10⁶ to about 2×10⁷, about 0.2×10⁶ to about 1×10⁷, about 0.2×10⁶ to about 1.5×10⁷, about 0.2×10⁶ to about 2×10⁷, or about 5×10⁶ cells.

The NK cells hereof administered to a subject can comprise about 1 million, about 2 million, about 3 million, about 4 million, about 5 million, about 6 million, about 7 million, about 8 million, about 9 million, about 10 million, about 11 million, about 12 million, about 12.5 million, about 13 million, about 14 million, or about 15 million cells. The cells can be administered as a single dose or multiple doses. The NK cells hereof can be administered in numbers of NK cells per kg of subject body weight.

### General

All patents, patent application publications, journal articles, textbooks, and other publications mentioned in the specification are indicative of the level of skill of those in the art to which the disclosure pertains.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. The terms and expressions which are employed, are used as terms of description and not of limitation. In this regard, where certain terms are defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Certain Definitions,” are used in the “Detailed Description,” such use is solely for ease of reference and is not intended to limit any disclosure made in one section to that section only; rather, any disclosure made under one subheading is intended to constitute a disclosure under each and every other subheading.

Although any methods and materials similar to or equivalent to those described herein can be used in the practice or testing of the subject of the present application, the preferred methods and materials are described herein.

When ranges are used herein for physical properties, such as molecular weight, or chemical properties, such as chemical formulae, all combinations and sub-combinations of ranges and specific embodiments therein are intended to be included.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

While the present disclosure has been made with reference to humans and human cells and genes, it is contemplated that hPSCs and NK cells can be generated from other species, such as other species of mammals, using cells and genes from that species. Such hPSCs and NK cells then can be used to treat members of that species in accordance with the teachings provided herein.

### Certain Definitions

The term “about,” when referring to a number or a numerical value or range (including, for example, whole numbers, fractions, and percentages), means that the number or numerical range referred to is an approximation within experimental variability (or within statistical experimental error), and thus the numerical value or range can vary between 1% and 15% of the stated number or numerical range (e.g., +/−5% to 15% of the recited value), provided that one of ordinary skill in the art would consider equivalent to the recited value (e.g., having the same function or result).

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

The term “receptor” refers to a chemical structure in biological systems that receives and transmits signals.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, “integration” means that one or more nucleotides of a construct is stably inserted into the cellular genome, i.e., covalently linked to the nucleic acid sequence within the cell's chromosomal DNA. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

As used herein, the term “exogenous” means that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, when used in reference to expression of an encoding nucleic acid, the term refers to expression of an encoding nucleic acid contained within the cell and not exogenously introduced.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

### EXAMPLES

The following examples serve to illustrate the present disclosure so as to provide those of ordinary skill in the art with a complete disclosure and description of how to make and use the embodiments hereof. The examples are not intended to limit the scope of the claimed invention in any way, nor are they intended to represent that the experiments below are all or the only experiments performed. Efforts have been made to ensure accuracy with respect to the numbers used (e.g., amounts, temperature, etc.), but some experimental errors and deviations should be accounted for. Unless otherwise indicated, parts are parts by weights, molecular weight is weight average molecular weight, temperature is in degrees Centigrade, and pressure is at or near atmospheric.

### Materials and Methods

Donor plasmid construction. To construct adeno-associated virus site 1 (AAVS1)-Puro XLone-NFIL3, spi-1 proto-oncogene (SPI1), and inducible inhibitor of DNA binding 2 (ID2) plasmids, fragments of human nuclear factor, interleukin 3 regulated (NFIL3), SPI1 and ID2 genes were amplified from Addgene plasmids (#82985, 97039, and 98394, respectively) and used to replace enhanced green fluorescent protein (eGFP) in the AAVS1-Puro XLone-eGFP donor plasmid (Addgene #136936).

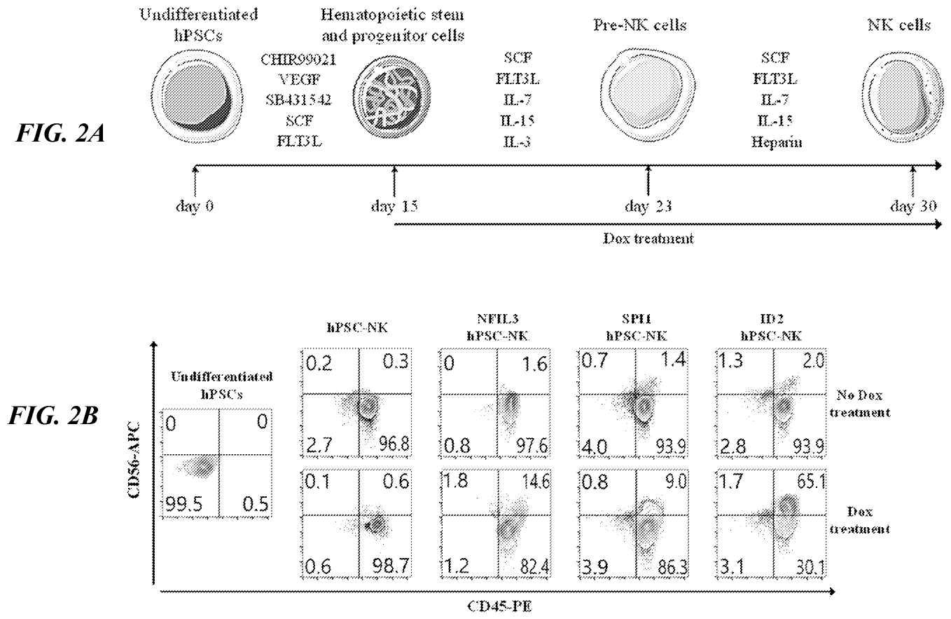

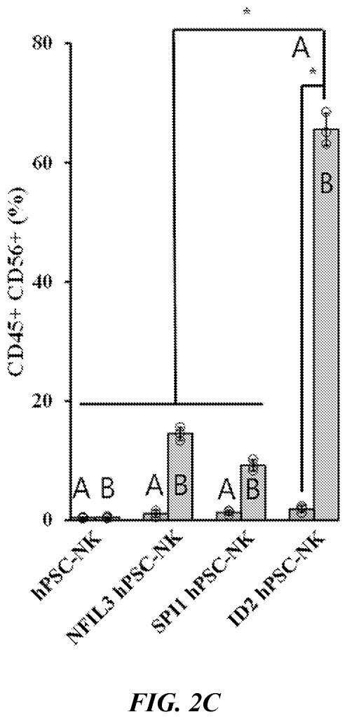

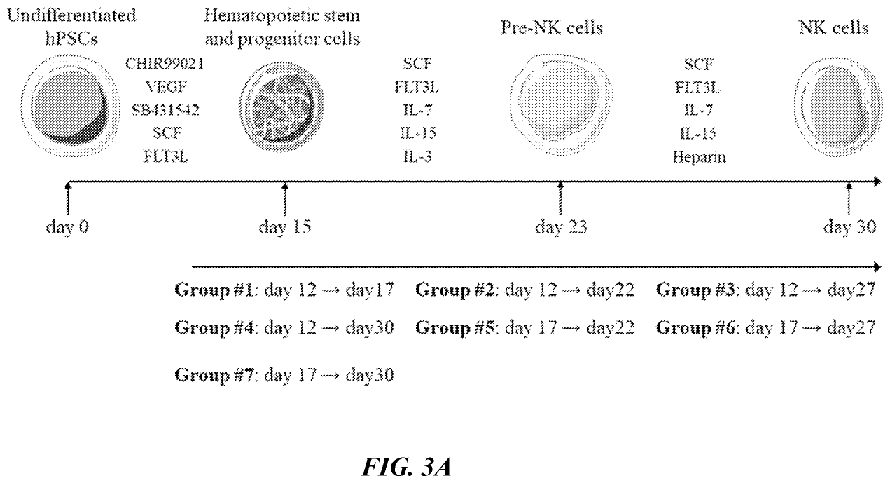

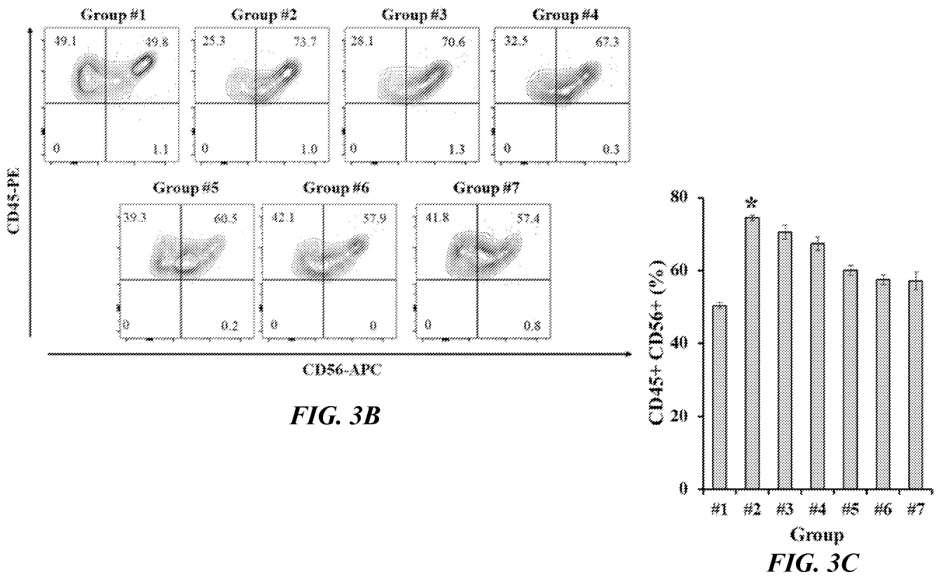

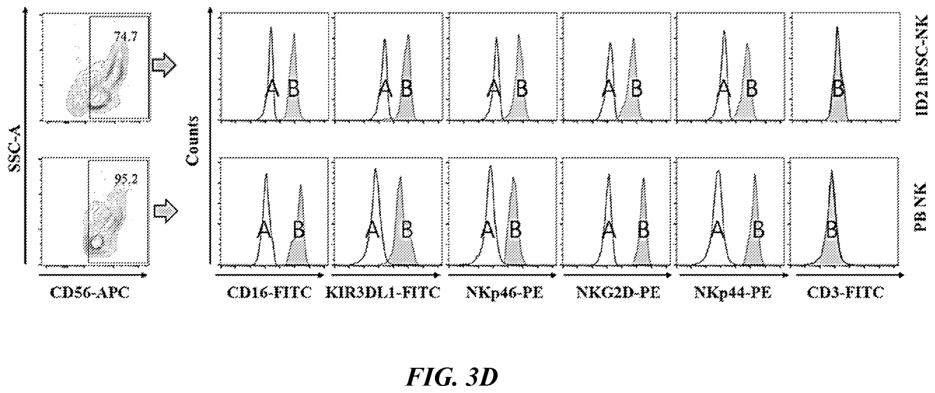

Maintenance and differentiation of hPSCs. H9 human pluripotent stem cells (hPSCs) were obtained from WiCell and maintained on Matrigel- or iMatrix-511-coated plates in mTeSR plus or E8 medium. (WiCell Research Institute, Inc., Madison, WI). For natural killer (NK) cell differentiation, hPSCs were dissociated with 0.5 mM ethylenediaminetetraacetic acid (EDTA) and seeded onto iMatrix-511-coated 24-well plate at a cell density between 10,000 and 80,000 cell/cm² in mTeSR plus medium with 5 NM Y27632 for 24 hours (day −1). Afterwards, NK cell differentiation was performed according to a previous report with modification as shown in FIGS. 2A and 3A. Romee et al. (2016), supra.

Briefly, 6 μM CHIR99021 was used to induce mesoderm differentiation from day 0 to day 2 in LaSR basal medium, followed by 10 μM SB431542, 50 ng/mL stem cell factor (SCF) and vascular endothelial growth factor (VEGF) treatment from day 2 to day 4. Tamada et al. (2012), supra. To induce hematopoiesis, 50 ng/mL SCF and FMS-like tyrosine kinase 3 ligand (FLT3L) were used in Stemline-II medium from day 4 to day 12. Ma et al., Versatile strategy for controlling the specificity and activity of engineered T cells, PNAS USA 113(4): E450-458 (2016). Floating day 12 hematopoietic stem and progenitor cells (HSPCs) were collected and treated with 50 ng/mL SCF, FLT3L, interleukin 3 (IL-3), interleukin 7 (IL-7), and interleukin 15 (IL-15) from day 15 to day 23. From day 23 to day 30, differentiated cultures were treated with 50 ng/mL SCF, FLT3L, IL-7, and IL-15 as well as 5 μg/mL heparin. For feeder layer-based NK cell differentiation, day 12 HSPCs were collected and transferred on OP9 stromal feeder cells, which were cultured in the α-MEM medium containing 20% fetal bovine serum (FBS), 10 ng/mL SCF, 10 ng/mL FLT3L, 5 ng/mL IL-7, and 10 ng/mL IL-15. Ma et al. (2021), supra. After co-culturing for 7 days, differentiated cells were collected and transferred on fresh OP9 feeder cells, and NK cell differentiation was continued for 4 weeks under the same conditions.

hPSC-NK cell purification. hPSC-derived NK cells were purified using EasySep™ FITC Positive Selection Kit (StemCell Technologies, Vancouver, Canada) according to the manufacturer's instructions. Briefly, differentiated NK cells were centrifuged at 200×g for 5 minutes, washed twice with 10 mL of PBS −/− solution containing 1% bovine serum albumin (BSA) (FlowBuffer-1), and then pelleted by centrifugation. After aspirating the supernatant, the cell pellet was resuspended in 100 μL FlowBuffer-1 at a cell concentration of 1×10⁸ cells/mL with 1:50 CD56-FITC antibody and incubated in the dark at room temperature for 30 minutes. Afterwards, cell and antibody mixtures were washed once with 2 mL of FlowBuffer-1 and incubated with 10 μL EasySep™ FITC Selection Cocktail in 100 μL FlowBuffer-1 at room temperature for 15 minutes. Five (5) L of well-mixed magnetic nanoparticles were then added to the 100 μL cell mixture, and the mixture was incubated at room temperature for another 10 minutes. The resulting cell suspension was then brought to a total volume of 2.5 mL FlowBuffer-1 in a flow tube, and the tube was placed into the magnet without a cap for 5 minutes. The magnet was then inverted in one continuous motion to pour off the supernatant and then returned to an upright position. The flow tube was moved from the magnet and washed with 2.5 mL FlowBuffer-1 to resuspend the cells on the flow tube wall by gently pipetting up and down two to three times. The magnet treatment was repeated two to three times, and the enriched NK cells were then resuspended in an appropriate amount of desired medium for further application.

Nucleofection and genotyping of hPSCs. To increase cell viability, hPSCs were treated with 10 μM Y27632 3-4 hours before nucleofection or overnight. Singularized hPSCs (1-2.5×10⁶) were nucleofected with 6 μg AAVS1 XLone donor plasmids along with 6 μg SpCas9 AAVS1 gRNA T2 (Addgene; #79888) in 100 μl human stem cell nucleofection solution (Lonza; #VAPH-5012) using program B-016 in a Nucleofector 2b. Nucleofected hPSCs were then plated into one well of a Matrigel-coated 6-well plate in 3 ml pre-warmed mTeSR plus with 10 μM Y27632. Twenty-four hours later, the medium was changed with mTeSR plus containing 5 μM Y27632, followed by a daily medium change. When cells reached about 80% confluency, 1 μg/ml puromycin (Puro) was applied for drug selection for about 1 week. Individual clones were then picked and expanded for 2-5 days in each well of a Matrigel-coated 96-well plate, followed by PCR genotyping using QuickExtract™ DNA Extraction Solution (Epicentre; #QE09050) and 2×GoTaq Green Master Mix (Promega; #7123). For positive genotyping, the following primer pair was used: CTGTTTCCCCTTCCCAGGCAGGTCC (SEQ ID NO: 1) and TCGTCGCGGGTGGCGAGGCGCACCG (SEQ ID NO: 2) (Tₘ=65° C.). For homozygous genotyping, the following set of primer sequences was used: CGGTTAATGTGGCTCTGGTT (SEQ ID NO: 3) and GAGAGAGATGGCTCCAGGAA (SEQ ID NO: 4) (Tₘ=60° C.).

Tumor cell line culture. U87MG, A549, LNCaP, and MDA-MB-231 tumor cells were kindly provided and cultured by the laboratories of Drs. Sandro Matosevic, Chang-Deng Hu and Philip Low at Purdue University. U87MG, A549, LNCaP, and MDA-MB-231 cells were cultured in Eagle's Minimum Essential Medium (EMEM) (containing 10% FBS, 100 units mL⁻¹ penicillin and 100 mg mL⁻¹ streptomycin), Kaighn's Modification of Ham's F-12 Medium (F-12K) (containing 10% FBS, 100 units mL⁻¹ penicillin and 100 mg mL⁻¹ streptomycin), RPMI-1640 Medium (containing 10% FBS, 100 units mL⁻¹ penicillin and 100 mg mL⁻¹ streptomycin), and Leibovitz's L-15 Medium (containing 10% FBS, 100 units mL⁻¹ penicillin and 100 mg mL⁻¹ streptomycin), respectively. All tumor cell lines were incubated at 37° C. in a humidified 5% CO₂ atmosphere. The medium was changed every two days, and cells were passaged at 70%-80% confluency.

Expansion of NK cells. Peripheral blood mononuclear cells (PBMCs) were isolated by Lymphoperp™ (StemCell Technology, 07851) gradient centrifugation in SepMate™ (StemCell Technology, 85450) tubes. Primary human cells were isolated from PBMCs by magnetic bead CD3 depletion (Miltenyi Biotec, 130-050-101), followed by CD56 (Miltenyi Biotec, 130-111-553) isolation. Purified NK cells were cultured in AIM-V (Invitrogen) medium with 500 U/mL IL-2 (PEPROTECH, 200-02), 2 ng/mL IL-15 (PEPROTECH, 200-15), and 100 ng/mL OKT3 (Ortho Pharmaceuticals, Raritan, NJ) at the concentration of 1×10⁶ cells/mL for 24 hours at 37° C. in a humidified 5% CO₂ atmosphere. The cells were cultured in AIM-V medium supplemented with interleukin 2 (IL-2) and IL-15 at 37° C. in a humidified 5% CO₂ atmosphere for further analysis.

Flow cytometry analysis. Differentiated cells were gently pipetted and filtered through a 70 or 100 m strainer sitting on a 50 mL tube. The cells were then pelleted by centrifugation and washed twice with PBS −/− solution containing 1% BSA. The cells were stained with appropriate conjugated antibodies (Table 1) for 25 minutes at room temperature in the dark and analyzed in an Accuri C6 plus cytometer (Beckton Dickinson, Franklin Lakes, NJ) after washing with BSA-containing PBS −/− solution. FlowJo software was used to process the flow data.

TABLE 1

| | | Concen- |
|---|---|---|
| Antibody | Source/Isotype/clone/cat. no. | tration |
| CD45-PE | BD Biosciences/Mouse IgG1/HI30/555483 | 1:50 |
| CD56-APC | BioLegend/Mouse IgG1/5.1H11/362503 | 1:50 |
| CD56-FITC | BioLegend/Mouse IgG1/5.1H11/362546 | 1:50 |
| SSEA-4 | Santa Cruz/Mouse IgG3/sc-21704 | 1:100 |
| OCT-3/4 | Santa Cruz/Mousc IgG2b/sc-5279 | 1:100 |
| CD16-FITC | BD Biosciences/Mouse IgG1/3G8/555406 | 1:50 |
| KIR3DL1-PE | BD Biosciences/Mouse IgG1/DX9/555964 | 1:50 |
| NKp46-PE | BD Biosciences/Mouse IgG1/9E2/557991 | 1:50 |
| NKp44-PE | BD Biosciences/Mouse IgG1/p44-8/558563 | 1:50 |
| NKG2D-PE | BD Biosciences/Mouse IgG1/1D11/557940 | 1:50 |
| CD107a-APC | BD Biosciences/Mouse IgG1/H4A3/560664 | 1:50 |
| IFNγ-PE | BD Biosciences/Mouse IgG1/B27/554701 | 1:50 |
| Actin- | Cytoskeleton/PHDG1 | 1:1000 |
| stain ™ 488 | | |
| Secondary | Alexa 488 Goat anti-Ms IgG1/A-21121 | 1:1,000 |
| Antibody | | |
| Secondary | Alexa 488 Goat anti-Rb IgG/A-11008 | 1:1,000 |
| Antibody | | |
| Secondary | Alexa 594 Goat anti-Ms IgG2b/A-21145 | 1:1,000 |
| Antibody | | |
| Secondary | Alexa 594 Goat anti-Ms IgG/A-21145 | 1:1,000 |
| Antibody | | |
| Secondary | Alexa 594 Goat anti-Rb IgG/A-11012 | 1:1,000 |
| Antibody | | |

Transwell migration analysis. For transwell assays, 600 μL of serum-free medium were placed in the lower chamber of a 24-well transwell plate (Corning Incorporated, Corning, NY). NK cells (2.5×10⁵) were added in 100 of serum-free medium to the upper chamber (5 m pore size), and the plate was incubated at 37° C. with 5% CO₂ for 5 hours. The number of NK cells that migrated to the lower chamber was determined by flow analysis (Accuri C6 plus cytometer; Beckton Dickinson, Franklin Lakes, NJ). Data are presented as percentage of migration based on total cell input.

NK cell-mediated in vitro cytotoxicity assay. The cell viability was analyzed by flow cytometry according to a previous protocol set forth in Lee et al., Regulation of CAR T cell-mediated cytokine release syndrome-like toxicity using low molecular weight adapters, Nature Communications 10: 2681 (2019). Briefly, tumor cells were stained with 2 μM Calcein-AM in MEM medium at 37° C. for 10 minutes in the dark, followed by 10% FBS treatment for 10 minutes in the dark at room temperature. Labeled tumor cells were pelleted at 300×g for 7 minutes and resuspended in culture medium with 10% FBS at a density of 50,000 cells/mL. Tumor cells (100 μL) were then mixed with 100 μL of 150,000, 250,000, and 500,000 cells/mL NK cells in 96 well plates and incubated at 37° C., 5% CO₂ for 12 hours.

To harvest all the cells, cell-containing media were transferred into a new round-bottom 96-well plate, and 50 μL of trypsin-EDTA were added to the empty wells to dissociate attached cells. After five minutes of incubation at 37° C., dissociated cells were transferred into the same wells of round-bottom 96-well plate with floating cells. All cells were pelleted by centrifuging (300×g, 4° C., 5 minutes) and washed with 200 μL of PBS−/− solution containing 0.5% BSA. The pelleted cells were stained with propidium iodide (PI) for 15 minutes at room temperature and analyzed in the Accuri C6 plus cytometer (Beckton Dickinson, Franklin Lakes, NJ).

Conjugate formation assay. To visualize immunological synapses. 100 μL of tumor cells (50,000 cells/mL) were seeded onto wells of 96-well plate and incubated at 37° C. for 12 hours, allowing them to attach. NK cells (100 μL; 500,000 cells/mL) were then added onto the target tumor cells and incubated for six hours before fixing with 4% paraformaldehyde (in PBS). Cytoskeleton staining was then performed using an F-actin Visualization Biochem Kit (Cytoskeleton Inc., Denver, CO).

Enzyme-linked immunosorbent assay (ELISA) analysis. To analyze the cytokine production by ELISA assay, 100 μL of tumor cells (50,000 cells/mL) were seeded onto wells of a 96-well plate and incubated at 37° C. for 12 hours, allowing them to attach. NK cells (100 μL; 500,000 cells/mL), with or without fluorescein isothiocyanate (FITC)-folate (10 nmol/L), were then added onto the target tumor cells and incubated for six hours. Afterwards, plates were centrifuged at 350×g for 10 minutes to spin down the cell debris, and 10 μL of top supernatant were collected for measuring TNFα and IL-6 production using an ELISA kit (ThermoFisher Scientific, US).

Statistical analysis. Three to five samples were analyzed for each group, and data are presented as mean±standard deviation (SD). Statistical significance was determined by Student's t-test (two-tail) between two groups, and three or more groups were analyzed by one-way analysis of variance (ANOVA). P<0.05 was considered statistically significant.

### Example 1

### Targeted Gene Knock-In in hPSCs Provided Inducible Expression of NFIL3, SPI1, and ID2

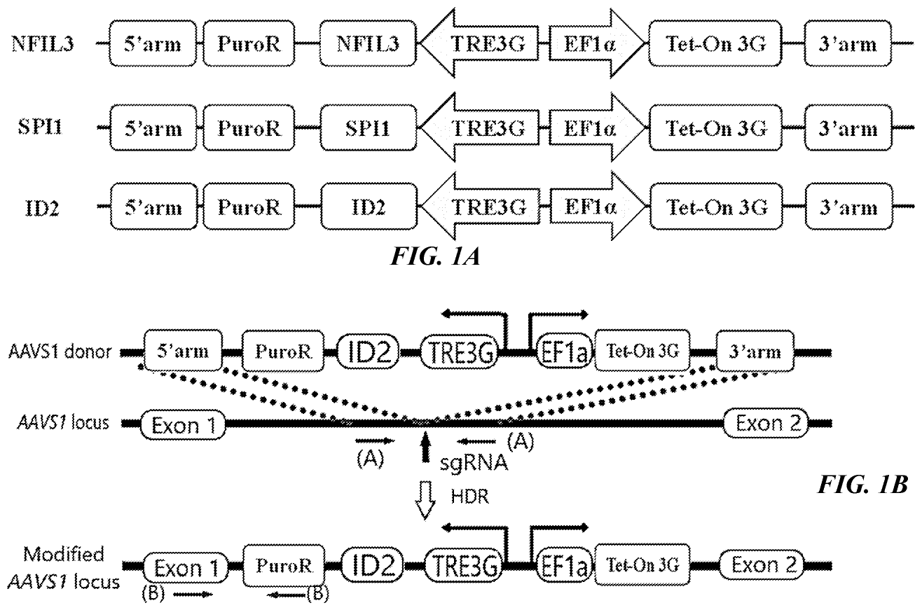

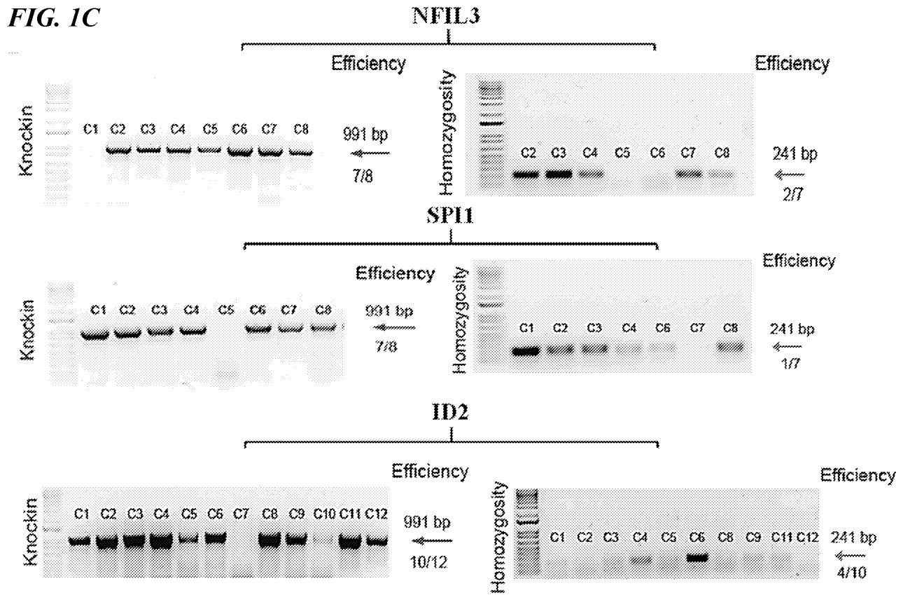

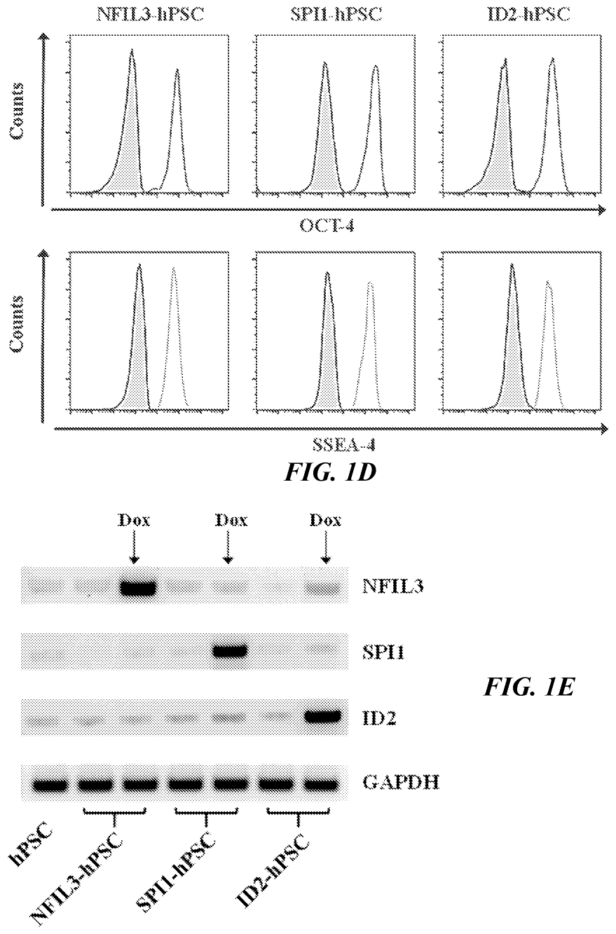

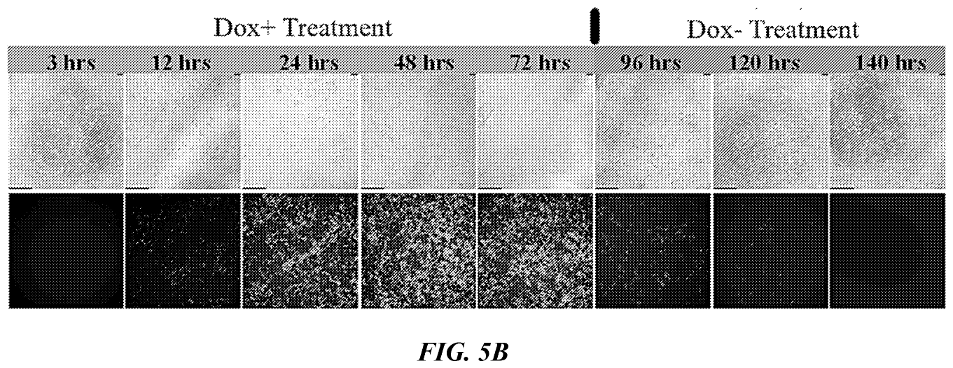

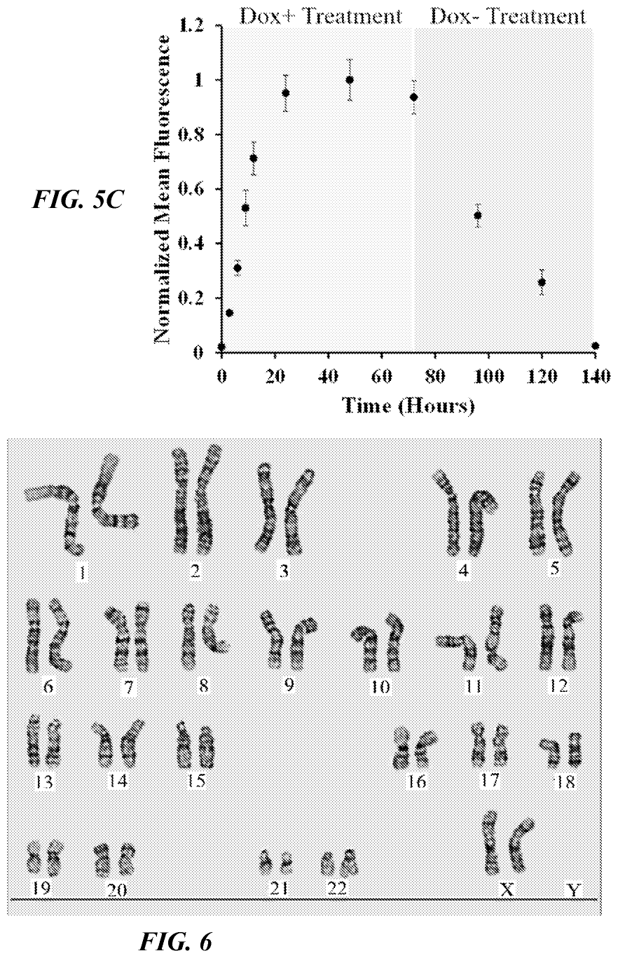

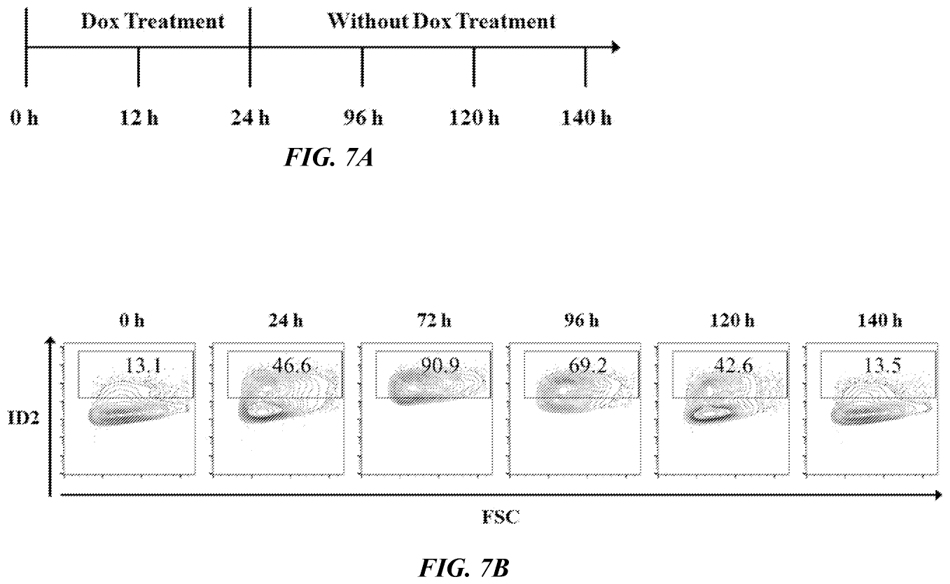

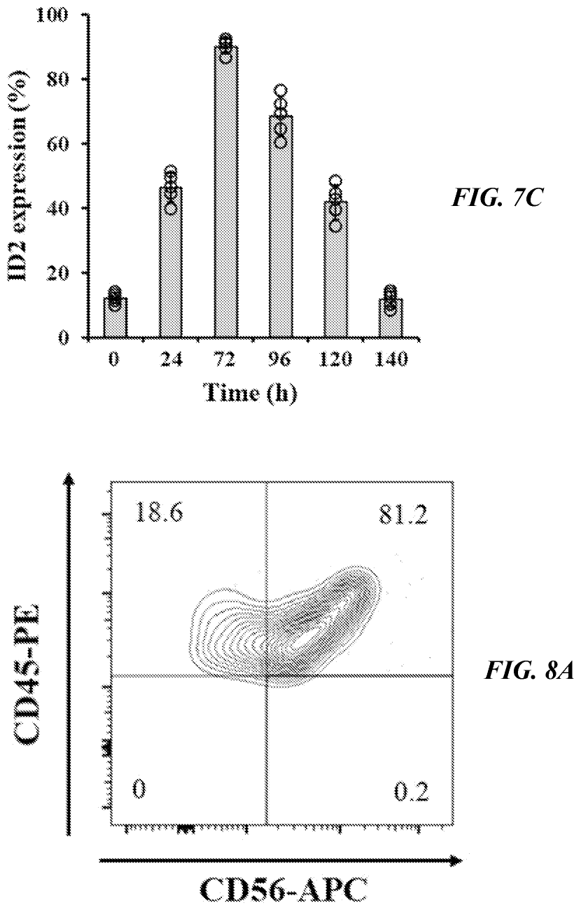

To temporally activate key transcription factors (TFs) in a manner representative of native NK cell development, an all-in-one, Tet-On 3G doxycycline-inducible expression system was employed, which contains two promoters, Tet-on 3G transactivator protein driven by the constitutive EF1α promoter, and a transgene of interest driven by the TRE3G promoter (FIGS. 1A and 5A). This all-in-one inducible system effectively expressed eGFP in H9 hPSCs under doxycycline (dox) treatment (FIGS. 5B-5C). eGFP was then replaced with NFIL3, SPI1, and ID2, and each of them was knocked into the endogenous AAVS1 safe harbor locus in H9 hPSCs via CRISPR/Cas9-mediated homology-directed repair (HDR) (FIG. 1B). After nucleofection, puromycin-resistant single cell-derived hPSC clones were isolated and subjected to PCR genotyping with a successful targeted knock-in efficiency of 87.5% (7 out of 8 clones), 87.5% (7 out of 8 clones), and 83.3% (10 out of 12 clones) (FIG. 1C) for NFIL3, SPI1, and ID2, respectively. The successfully targeted clones were then subjected to homozygosity assay, and 28.5% (2 out of 7 clones), 14.3% (1 out of 7 clones), and 40% (4 out of 10 clones) of NFIL3, SPI1, and ID2 knockin clones were homozygous (FIG. 1C). Heterozygous C7, C8, and C6 of NFIL3, SPI1, and ID2 knockin hPSCs were selected for NK cell differentiation. Genetically modified hPSCs displayed strong expression of the pluripotency markers stage-specific embryonic antigen-4 (SSEA-4) and octamer-binding transcription factor-4 (OCT4) (FIG. 1D). Importantly, these hPSCs retained a normal karyotype after CRISPR/Cas9-mediated genome editing (FIG. 6). Similar to inducible eGFP expression, the resulting knockin hPSCs expressed high levels of NFIL3, SPI, and ID2 in response to dox treatment (FIGS. 1E and 7A-7C).

### Example 2

### Overexpression of ID2 Promoted NK Cell Differentiation from hPSCs

To investigate the function of NFIL3, SPI1, and ID2 during in vitro NK cell development, a previous chemically-defined NK cell differentiation protocol was adapted and modified (FIG. 2A). Romee et al. (2016), supra. Under dox treatment during the whole differentiation, about 0.6%, 14.6%, 9.0%, and 65.1% CD45+CD56+ cells were generated for wild-type hPSCs, NFIL3-hPSCs, SPI1-hPSCs, and ID2-hPSCs, respectively (FIG. 2B), suggesting that overexpression of NK-specific TFs improves in vitro NK cell differentiation from hPSCs. Notably, forced expression of ID2 yielded the highest percentage of CD45+CD56+ cells under the chemically-defined, feeder-free monolayer culture condition, consistent with enhanced ID2 expression during NK cell differentiation from hPSCs. Ma et al. (2022), supra; Mishra et al. (2012), supra. Collectively, the results support the use of forced TF expression in enhancing NK cell differentiation from hPSCs.

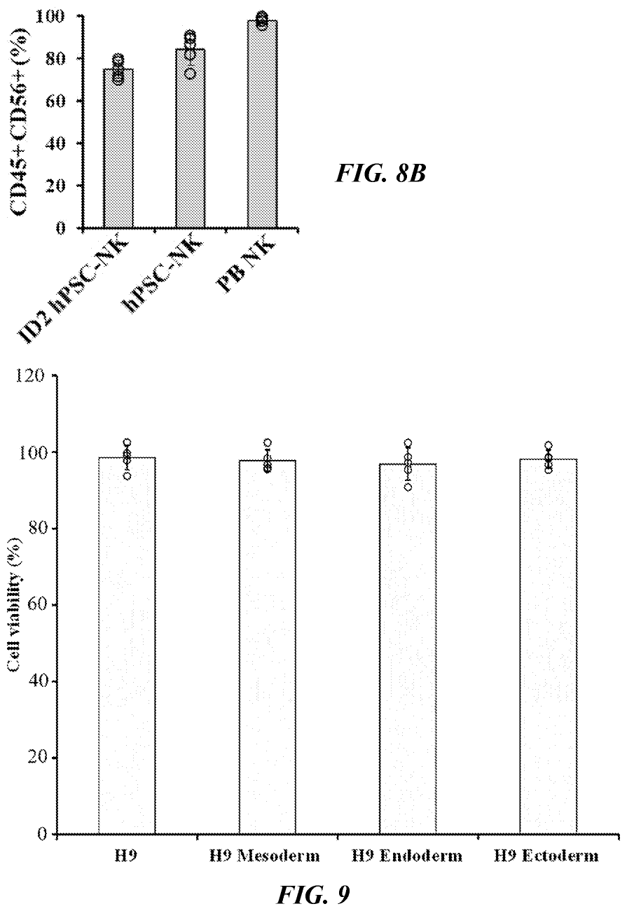

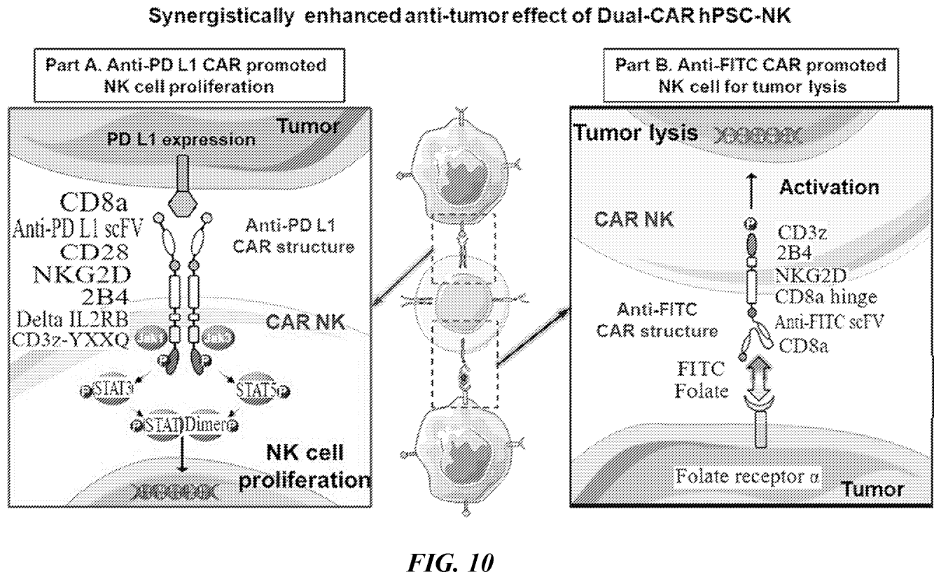

ID2 plays stage-specific functions during NK cell development and maturation in vivo. Chen et al. (2018), supra; Jiang et al. (2019), supra; Li et al. (2018), supra. Thus, the stage-specific effects of forced ID2 expression in NK cell generation from hPSCs was investigated to develop an optimized differentiation protocol (FIG. 3A). Temporal treatment of dox significantly affected the generation of CD45+CD56+ cells (FIGS. 3B-3C), confirming the stage-specific roles of ID2 during NK cell development. Among all tested conditions, dox treatment from day 12 to day 22 (Group #2) yielded the highest percentage (˜73.7%) of CD45+CD56+ cells (FIGS. 3B-3C). The resulting cells from optimized condition were further characterized, and they displayed high levels of typical NK cell-surface markers, including CD16, KIR3DL1, NKp46, NKG2D, and NKp44 (FIG. 3D), consistent with previously reported hPSC-derived NK cells (FIG. 8). Romee et al. (2016), supra; Cerwenka & Lanier (2016), supra; Cooper et al (2009), supra; Liu et al. (2018), supra. Taken together, the results demonstrated stage-specific roles of ID2 overexpression in enhancing NK cell differentiation from hPSCs.

### Example 3

### hPSC-Derived NK Cells Displayed Cytotoxicity Against Cancer Cells

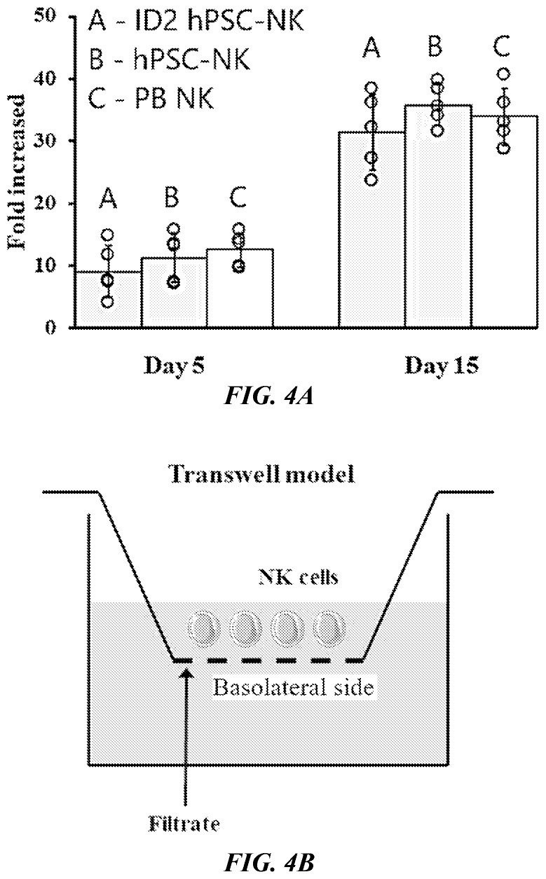

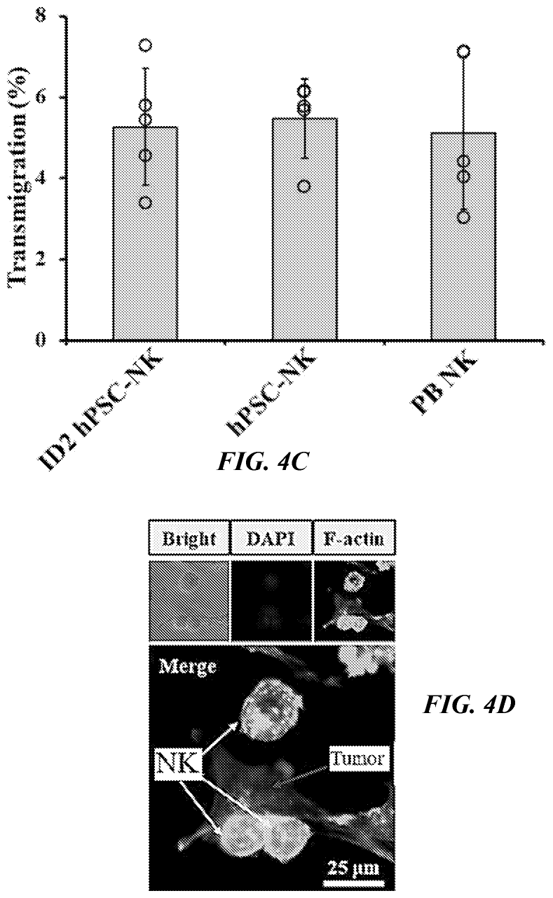

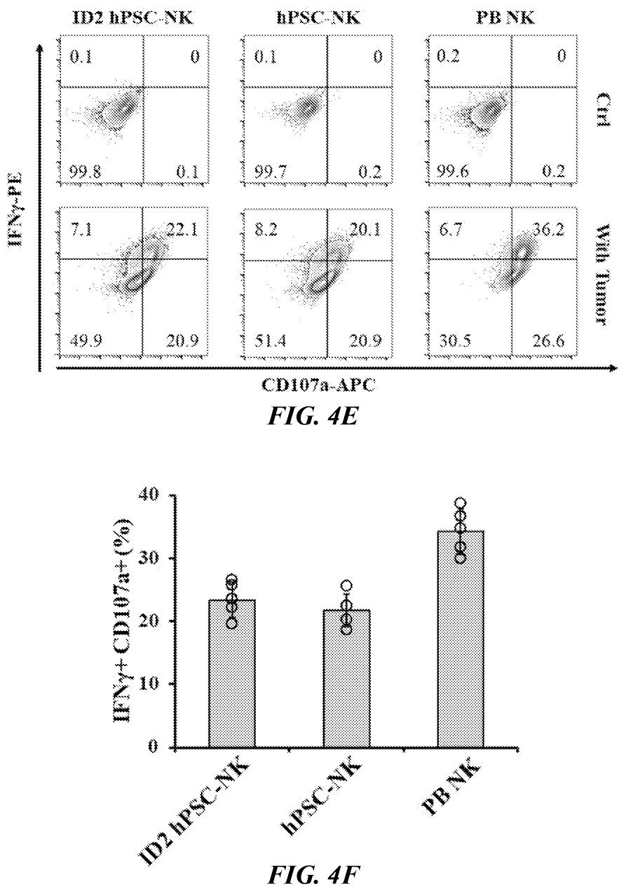

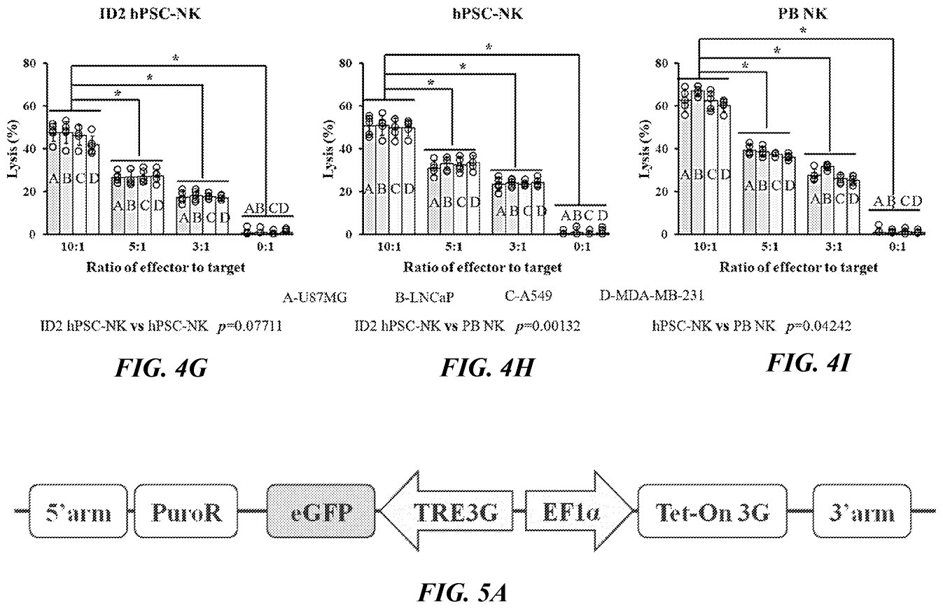

The expansion and transmigration ability of hPSC-derived NK cells were investigated. A similar expansion fold was observed in hPSC-derived and primary NK cells (FIG. 4A) in the presence of IL-2, IL-15, and OKT3. Seo et al. (2017), supra. Furthermore, ID2 overexpression-induced hPSC-NK cells exhibited similar transmigration ability as wild-type hPSC-derived and primary NK cells (FIGS. 4B-4C). To further explore their potential in cancer immunotherapy, ID2 overexpression-induced NK cells were co-cultured with different cancer cells for tumor-killing analysis. Two hours following co-culture with U87MG glioblastoma cells, immunological synapses were formed between NK and tumor cells (FIG. 4D), facilitating cytotoxicity activities of NK cells against tumor cells. As expected, hPSC-derived NK cells via ID2 overexpression or feeder layer co-culture expressed IFN-γ and CD107a in response to tumor cells (FIGS. 4E-4F), indicating cytotoxic granule release. The tumor-killing ability of hPSC-derived NK cells against different tumor cells, including LNCaP, A549, U87MG, and MDA-MB-231, was assessed and was similar to the tumor-killing ability of their counterparts in peripheral blood. hPSC-derived NK cells displayed a broad anti-tumor cytotoxicity at various effector-to-target ratios (FIGS. 4G-4I). Notably, hPSC-derived NK cells did not kill normal H9-derived somatic cells (FIG. 9); such data support their safety in future clinical application.

Adoptive NK cell-based immunotherapies hold great promise for clinical cancer treatment, given their unique innate tumor-killing ability and safety in allogeneic transplantation. In order to meet clinical needs (10⁷ cells/kg for a patient), several human sources of NK cells, including peripheral blood (PB) and umbilical cord blood (UCB), have been investigated in cancer immunotherapy. Du et al. (2021), supra. Primary NK cells isolated from PB and UCB sources, however, were heterogeneous, and these cells were not sufficient to treat many patients. Id.; Judge et al. (2020), supra; Jiang et al. (2019), supra. In contrast, hPSCs can be expanded unlimitedly and differentiated into NK cells to meet the clinical needs, providing a realistic, universal cell source for various therapies, such as cancer immunotherapy (e.g., targeted cancer immunotherapy). Sun et al. (2009), supra; Ma et al. (2022), supra.

A TF-mediated forward programming approach has been recently used to efficiently differentiate hPSCs into neural, glial, liver, skeletal and cardiac muscle cells. Luo et al. (2022), supra. However, such an approach has not yet been applied to NK cell induction. Here, hPSCs were genetically engineered with doxycycline-inducible expression of NFIL3, SPI1, and ID2, and TF-mediated forward programming enhanced NK cell differentiation, in which inducible ID2 expression yielded the highest percentage of CD45+CD56+ NK cells. This result is consistent with enhanced ID2 expression during NK cell differentiation from hPSCs. Ma et al. (2022), supra; Mishra et al. (2012), supra. The resulting hPSC-derived NK cells also displayed NK-specific surface markers and cytotoxic activities against various tumor cells in vitro.

In summary, the all-in-one inducible expression system can serve as a modular strategy to screen more transcription factors for robust NK or T cell induction from hPSCs. The engineered ID2-expressing hPSCs can be used to generate universal NK cells as potential standardized cellular products for clinical applications in cancer treatment.

### Example 4

### Screening CAR Structures with Enhanced NK Cell-Mediated Tumor-Killing Activities

Based on previous CAR constructs used in T and NK cells, eight different CARs, which were optimized for antitumor cytotoxicity and proliferation in NK-92 cells, were designed and evaluated (FIG. 2A).

CAR plasmid construction. Generally, to construct anti-PD-L1 lentiviral vectors, a DNA sequence encoding CD8a signal peptide, anti-PD-L1 nanobody, CD28 extracellular domain, CD28 or NKG2D transmembrane domain, CD28 or 2B4 intracellular co-stimulatory domain, ΔIL-2Rβ, and CD3ζ-YXXQ was directly synthesized and cloned into the lenti-luciferase-P2A-NeoR (Addgene #105621) backbone via NEBuilder HiFi DNA Assembly after Bam HI and Mlu I digestion. Zhang et al., Structural basis of a novel PD-L1 nanobody for immune checkpoint blockade, Cell Discovery 3: 17004 (2017).

For lentivirus production generally, 293TN cells were incubated in DMEM medium containing 10% FBS, 1% sodium pyruvate, and 0.5% GlutaMAX until 95-100% confluence. 4.5 μg lentiviral CAR plasmid, 3.0 μg psPAX2, and 1.5 μg pMD2.G were added to 450 μL of Opti-MEM medium and incubated at room temperature for five minutes. FuGENE HD reagent (27 μL) was then added to the mixture and incubated at room temperature for another 15 minutes. The resulting 450 μL plasmid mixture was added to 3 mL of culture medium and evenly distributed to three wells of a 6-well plate with 293TN cells after aspirating the old medium. Eighteen hours after plasmid addition, the medium from each well was aspirated and replaced with 3 mL of fresh culture medium and incubated for another 24 hours. Virus-containing supernatant was then collected every day with fresh warm medium change for 2 to 3 days, transferred to a 50 mL conical tube, and stored at 4° C. The resulting virus supernatant was then centrifuged at 2,000 g at 4° C. for 5 minutes or filtered through a 0.45 μm filter to remove cell debris.

The resulting anti-PD-L1 plasmids were then sequenced and digested with Mlu I to incorporate further an IRES-NeoR or IRES-GFP sequence. The anti-FITC CAR plasmid with CD8a signal peptide, anti-fluorescein single-chain variable fragment (scFv), CD8a extracellular and intracellular domains, 4-1BB co-stimulatory domain and CD3ζ signaling domain was previously constructed by the present investigators and cloned into their AAVS1-Puro CAG FUCCI donor plasmid (Addgene #136934). Lee et al. (2019), supra; Chang et al., Fluorescent indicators for continuous and lineage-specific reporting of cell-cycle phases in human pluripotent stem cells, Biotechnology & Bioengineering 117(7): 2177-2186 (2020).

The resulting AAVS1-Puro CAG anti-FITC-CAR plasmid was digested with Sgr DI and Mlu I and ligated to the lentiviral anti-PD-L1 CAR backbone to construct the lentiviral anti-FITC CAR vector. To make anti-FITC CAR plasmid with NKG2D transmembrane and 2B4 co-stimulatory domains, anti-FITC scFv sequence and a chimeric sequence of NKG2D, 2B4 and CD3ζ were PCR-amplified from lentiviral anti-FITC CAR vector and AAVS1-Puro CAG CLTX-NKG2D-2B4-CD3z CAR (Addgene #157744), respectively, and cloned into AAVS1-Puro CAG FUCCI plasmid via NEBuilder HiFi DNA Assembly to make AAVS1-Puro CAG anti-FITC-NKG2D-2B4-CD3z CAR, which was digested with Sgr DI and Mlu I, and ligated to the lentiviral anti-PD-L1 CAR backbone to construct the lentiviral anti-FITC-NKG2D-2B4-CD3z CAR.

CARs #1 to #4 were single antigen-targeting CARs against either PD-L1 or FITC using NK or T cell-specific signaling domains, and CARs #5 to #8 were combinatory dual antigen-targeting CARs. For the switchable anti-FITC scFv CARs, CARs #1, #5, and #7 employed an NK-specific transmembrane domain NKG2D, a co-stimulatory domain 2B4 and an intracellular domain CD3ζ, whereas CARs #2, #6, and #8 differed in the transmembrane domain CD8 and co-stimulatory domain 4-1BB. Fortumor microenvironment responsive anti-PD-L1 nanobody CARs, CARs #3, #5, and #7 used a NK-specific transmembrane domain NKG2D, a co-stimulatory domain 2B4, a truncated IL-2 receptor β-chain (Delta IL-2RB), an intracellular domain CD3ζ, and a STAT3-binding tyrosine-X-X-glutamine (YXXQ) motif, whereas CARs #4, #6, and #8 differ in the transmembrane domain CD28.

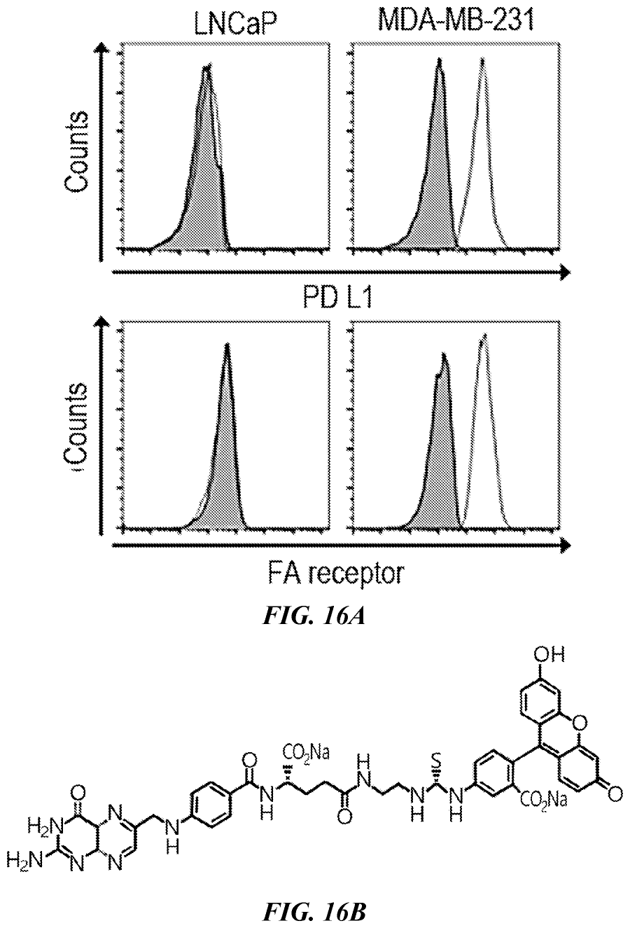

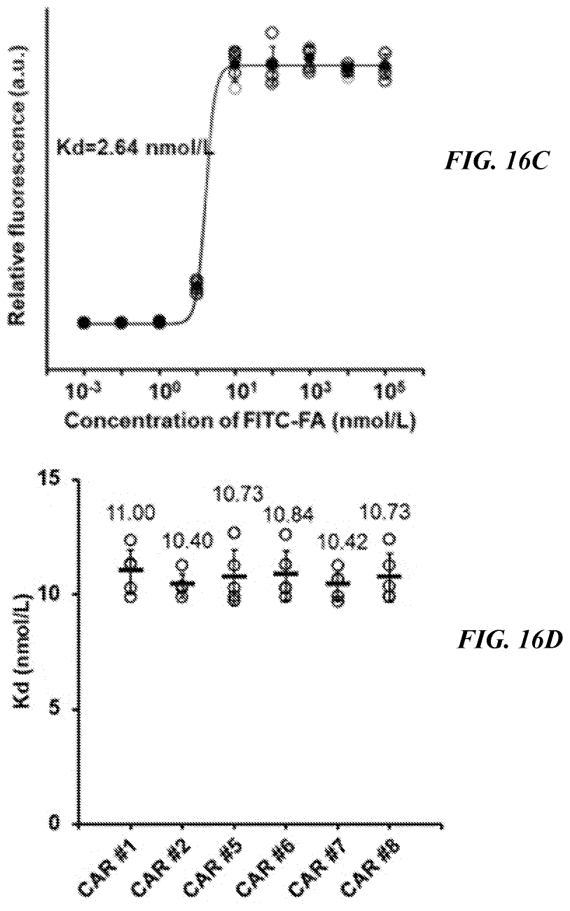

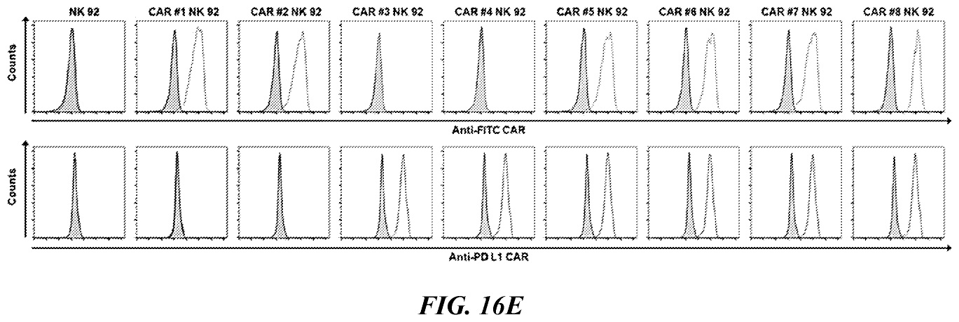

These CAR constructs were first tested in NK-92 cells for their ability to enhance antitumor activities against FRα+ and PD-L1+ tumor cells. Human breast cancer MDA-MB-231 cells express high levels of FRα and PD-L1, whereas human prostate adenocarcinoma LNCaP cells express neither FRα nor PD-L1 (FIG. 16A). Martin et al., Paucity of PD-L1 expression in prostate cancer: Innate and adaptive immune resistance, Prostate Cancer & Prostatic Diseases 18: 325-332 (2015). These two tumor lines were used for antitumor cytotoxicity analysis of our engineered CAR NK-92 cells.

Regarding the NK-92 cells and lentiviral transduction, generally, NK-92 cells were cultured in MyeloCult H5100 medium containing 100 units/mL human recombinant IL-2. For lentiviral transduction, NK-92 cells were first stimulated by IL-2 and IL-15. Briefly, the NK-92 cells were counted and resuspended in appropriate medium (RPMI 1640, 10% FBS, 2 nM L-glutamine, 20 ng/mL IL-2, 50 ng/mL IL-15, and 100 ng/mL IL-12) at 1×10⁶ cells/mL. These NK-92 cells were stimulated for two hours before lentiviral transduction.

After cytokine stimulation, 1×10⁵ NK-92 cells were plated in each well of a 12-well plate, and cells were treated with 1 mL virus supernatant and polybrene (8 μg/mL) overnight at 37° C., 5% CO₂. After 24 hours, viruses were removed by centrifuging at 360×g for five minutes, and the resulting NK-92 cells were suspended in 1 mL MyeloCult H5100 medium with 100 units/mL human recombinant IL-2. After five days, transduced NK-92 cells were centrifuged at 360×g for five minutes and resuspended in 1 mL MyeloCult H5100 medium containing 100 units/mL human recombinant IL-2 and 1 μg/mL puromycin or 100 μg/ml G418. At least 8-day drug screening is needed to enrich successfully transduced NK-92 cells.

Regarding MDA-MB-231 and LNCaP cell maintenance, LNCaP tumor cells were kindly provided and cultured by the laboratory of Dr. Chang-Deng Hu at Purdue University. MDA-MB-231 cells were cultured in Leibovitz's L-15 medium (containing 10% FBS, 100 units mL⁻¹ penicillin and 100 mg mL⁻¹ streptomycin), and LNCaP cells were cultured in RPMI-1640 medium (containing 10% FBS, 100 units mL⁻¹ penicillin and 100 mg mL⁻¹ streptomycin). These two cell lines were incubated at 37° C., 5% CO₂. The culture medium was changed every two days and cells were passaged at 70-80% confluency.

Bi-specific FITC-folate adapter was first synthesized with folic acid on the left side for binding FRα on breast tumor cells and fluorescein on the right side for anti-FITC CAR targeting (FIG. 16B). Lee et al. (2019), supra. The binding affinity (K_{d}) of FITC-folate for MDA-MB-231 tumor cells was measured as 2.64 nM (FIG. 16C), and the binding affinity (K_{d}) of FITC-folate for various anti-FITC CAR NK-92 cells were about 10 nM (FIG. 16D). Considering that insufficient intracellular bridges will be formed at very low FITC-folate concentration, whereas at very high concentrations, intracellular bridging will be locked due to monovalent saturation of ligand binding sites on both cell types with excess FITC-folate adapters, 10 nM of FITC-folate was used in the following studies.

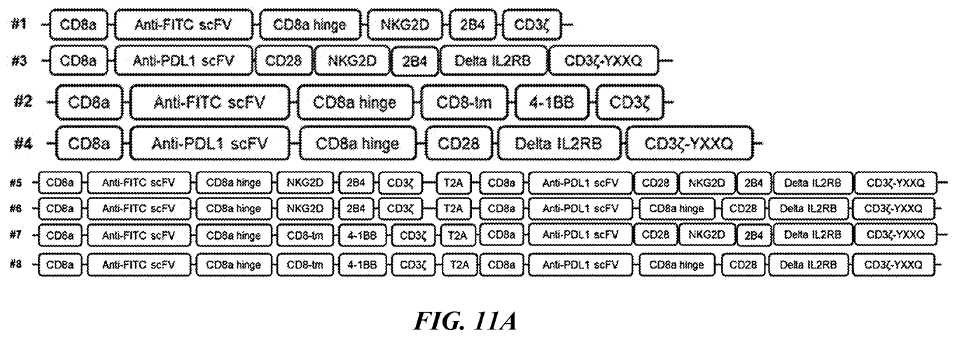

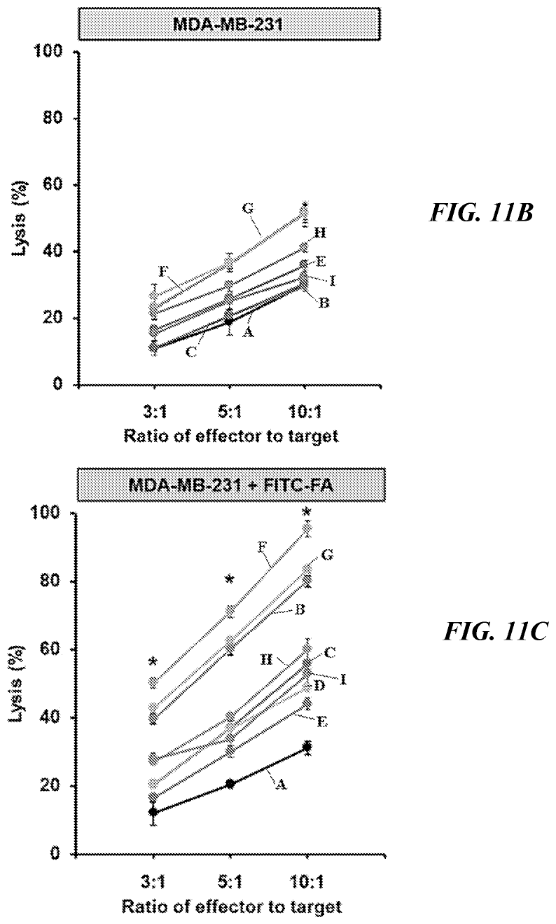

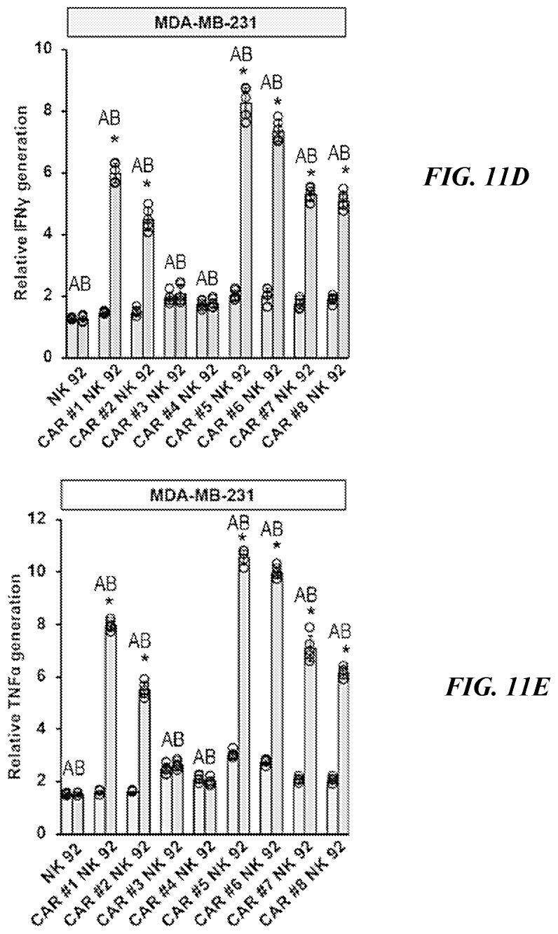

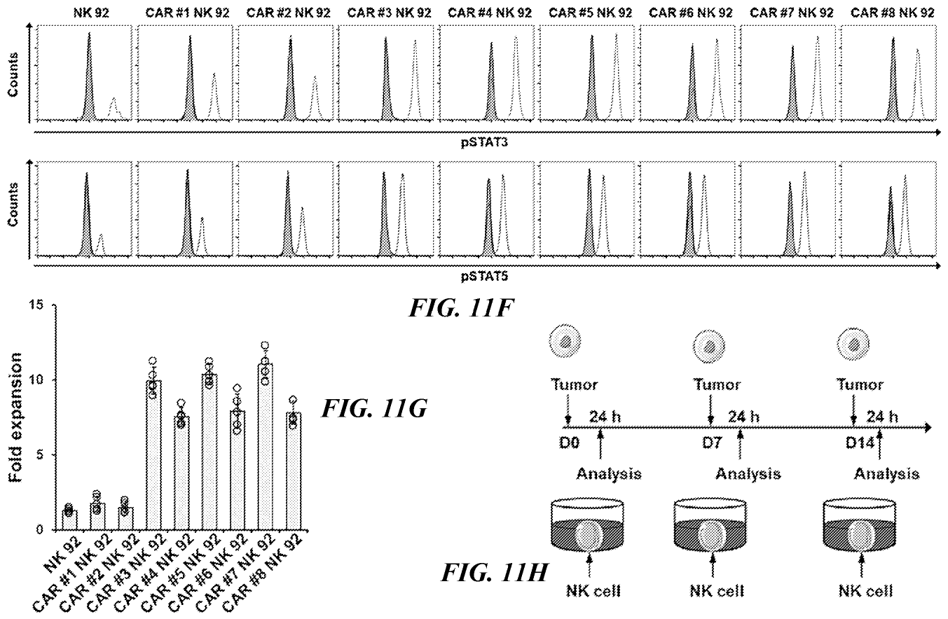

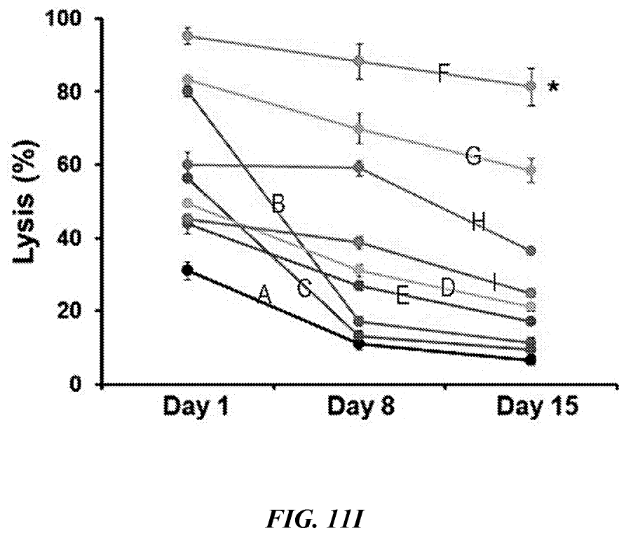

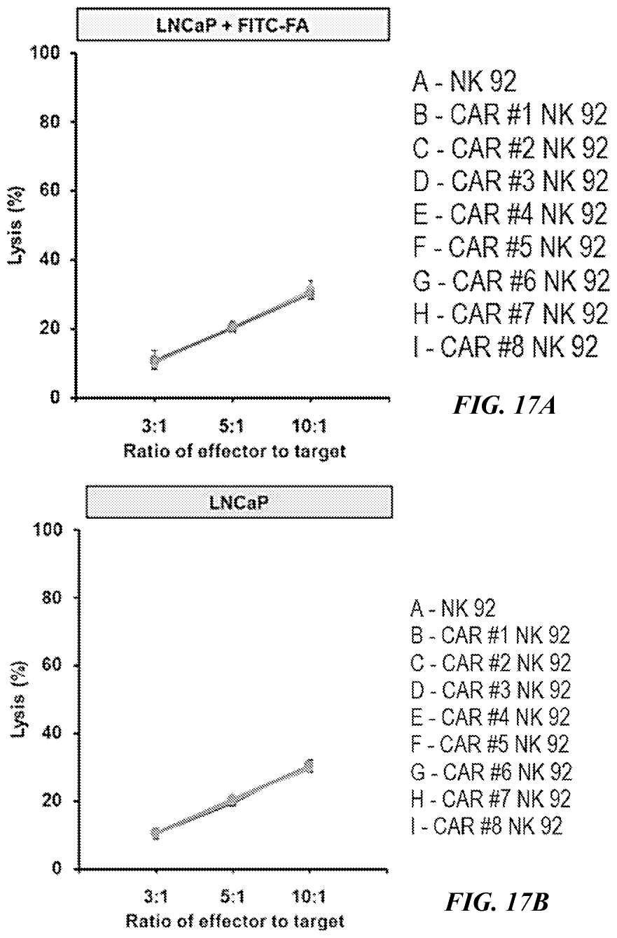

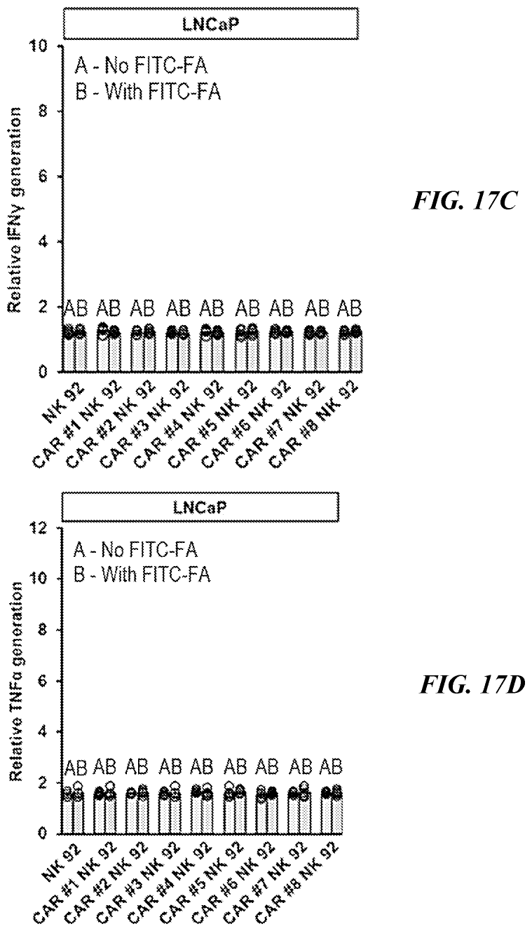

The killing potency of various anti-FITC and/or anti-PD-L1 CAR NK-92 cells (FIG. 16E) were then tested in MDA-MB-231 (FRα+PD-L1+) and LNCaP cell (FRα− PD-L1−) cells. As expected, CAR-expressing NK-92 cells exhibited more potent cytotoxicity against MDA-MB-231 and more cytotoxic granule release than LNCaP cells (FIG. 11B and FIGS. 17A-17D). In the presence of bi-specific FITC-folate adapter, anti-MDA-MB-231 cytotoxicity of CAR NK-92 cells was significantly increased (FIG. 11C), indicating the specificity of the anti-FITC CAR. Among these CARs, CARs #1, #5, and #6 displayed a much larger increase of anti-tumor activity in NK-92 cells against FRα+PD-L1+ breast cancer cells after bridging with the FITC-folate adapter (FIG. 11C), along with significantly enhanced IFNγ and TNFα release (cytotoxic granule) (FIGS. 11D-11E). As expected, these FITC-folate bridged NK-CARs (#1, #5, and #6) mediated higher killing potency in NK-92 cells than T cell-specific CARs (#2, #7, and #8).

### Example 5

### Screening CAR Structures with Enhanced NK Cell Proliferation Activity

The capability of various CARs to promote antigen-specific NK cell proliferation after co-culturing with tumor cells was evaluated. Both truncated IL-2 receptor β-chain (ΔIL-2RB) and the STAT3-binding tyrosine-X-X-glutamine (YXXQ) motif in the anti-PD-L1 CARs were designed to enhance cell proliferation and persistence via activation of JAK, STAT3 and STAT5 signaling pathways. Kagoya et al., A novel chimeric antigen receptor containing a JAK-STAT signaling domain mediates superior antitumor effects, Nature Medicine 24: 352-359 (2018).

Cell viability was analyzed by flow cytometry according to a previous protocol described in Kandarian et al., A flow cytometry-based cytotoxicity assay for the assessment of human NK cell activity, JoVE: Immunology & Infection 2017 (2017). Briefly, tumor cells were stained with 2 μM Calcein-AM in MEM medium at 37° C. for 10 minutes in the dark, followed by 10% FBS treatment for 10 minutes in the dark at room temperature. Labeled tumor cells were pelleted at 300×g for 7 minutes and resuspended in culture medium with 10% FBS at a density of 50,000 cells/mL. 100 μL of tumor cells were then mixed with 100 μL of 150,000, 250,000, and 500,000 cells/mL NK cells in 96-well plate with or without the antigen to be tested (e.g., FITC-folate (10 nmol/L)) and incubated at 37° C., 5% CO₂, for 12 hours.

To harvest all the cells, cell-containing medium was firstly transferred into a new round-bottom 96-well plate, and 50 μL trypsin-EDTA were added to the empty wells to dissociate attached cells. After a five-minute incubation at 37° C., dissociated cells were transferred into the same wells of round-bottom 96-well plate with floating cells. All cells were pelleted by centrifuging (300×g, 4° C., 5 minutes) and washed with 200 μL of PBS−/− solution containing 0.5% BSA. The pelleted cells were stained with propidium iodide (PI) for 15 minutes at room temperature and analyzed in the Accuri C6 plus cytometer (Beckton Dickinson, Franklin Lakes, NJ).

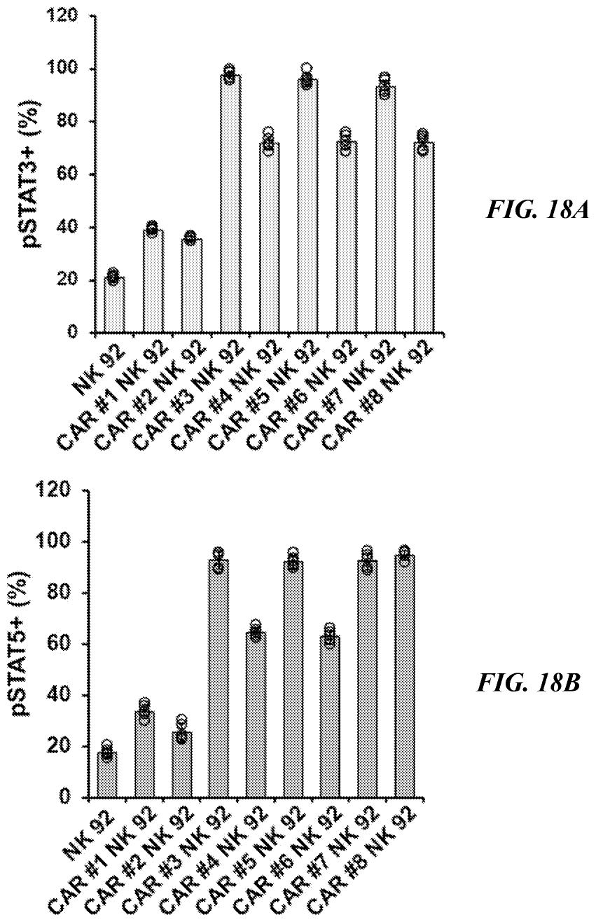

Upon PD-L1+ MDA-MB-231 cell stimulation, CAR NK-92 cells exhibited upregulated levels of phosphorylated STAT3 (pSTAT3) and pSTAT5 (FIG. 11F), among which CARs #3, #5, and #7 displayed superior ability in upregulating pSTAT3 and pSTAT5 (FIGS. 18A-18B). As expected, CARs #3, #5, and #7 also promoted greatest proliferation in NK-92 cells (FIG. 11G).

A continuous in vitro tumor cell exposure model was constructed to investigate the persistence and memory-like phenotype of NK-92 cells after CAR-engineering (FIG. 11H). Consistent with previous observation, NK-92 cells engineered with CARs #1, #5 and #6 displayed superior tumor-killing ability against FRα+PD-L1+ breast cancer cells under the initial antigen exposure at day 1 (FIG. 11I).

As the antigen exposure increased (days 8 and 15), a significant reduction of anti-tumor cytotoxicity was observed in NK-92 cells with anti-FITC CAR only (CAR #1), whereas dual anti-FITC and anti-PD-L1 CAR NK-92 cells (CAR #5 and #6) still exhibited excellent anti-tumor activities at day 15. Notably, dual anti-FITC and anti-PD-L1 CAR #5 with NK-specific transmembrane and co-stimulatory domains presented superior persistence as compared to all other CARs. The results support that dual CAR design can synergize multi-functions of NK cells under specific tumor antigen stimulation and achieve superior antitumor activities and persistence under a complex tumor microenvironment.

### Example 6

### hPSC Transduction

hPSCs can be engineered to express CAR construct(s) using lentiviral transduction strategies for functional CAR-NK cell production. For hPSC transduction, hPSCs were dissociated with 0.5 mM EDTA and seeded onto iMatrix 511-coated 6-well plate at a cell density between 10,000 and 80,000 cells/cm² in mTesR plus medium with 5 VM Y27632. Twenty-four hours later, the stem cell culture medium was aspirated and replaced with 1 mL of mTesR plus medium with 5 μM Y27632 and 1 mL of virus supernatant, which were removed and replaced with 2 mL of fresh mTeSR plus after 24 hours. Two to three days after transduction, 100 μg/ml G418 or 1 μg/mL puromycin was applied to select successfully transduced hPSCs. To further enrich desired cells, transduced hPSCs were dissociated and transferred to 96-well plate at a cell density of 10 cells/mL. After a 4-day culture, hPSCs were continuously treated with 100 μg/ml G418 or 1 μg/mL puromycin for 8 more days.

### Example 7

### Engineering hPSC-Derived NK Cells with Dual CARs for Enhanced Function

Given its superior anti-tumor activity and persistence in NK-92 cells, dual anti-FITC, and PD-L1 CAR #5 was selected for CAR engineering of hPSC-derived NK cells. Single antigen-targeting anti-FITC CAR #1 and anti-PD-L1 CAR #3 were used as controls for anti-tumor cytotoxicity and cell proliferation, respectively. To provide a potentially universal source of CAR-expressing NK cells, hPSCs were engineered with these three CARs.

Briefly, the H9 hPSC line was obtained from WiCell and maintained on Matrigel-coated plates in mTeSR plus medium. For NK cell differentiation, hPSCs were dissociated with 0.5 mM EDTA and seeded onto iMatrix 511-coated 24-well plate at a cell density between 10,000 and 80,000 cells/cm² in mTesR plus medium with 5 μM Y27632 for 24 hours (day −1). At day zero, cells were treated with 6 μM CHIR99021 (CHIR) in Dulbecco's Modified Eagle's Medium (DMEM) medium supplemented with 100 μg/mL ascorbic acid (DMEM/Vc), followed by a medium change with LaSR basal medium from day one to day four. VEGF (50 ng/mL) was added to the medium from day two to day four. At day four, medium was replaced by Stemline II medium (Sigma-Aldrich, St. Louis, MO) supplemented with 10 μM SB431542, 25 ng/mL SCF and FLT3L. On day six, SB431542-containing medium was aspirated, and cells were maintained in Stemline II medium with 50 ng/mL SCF and FLT3L. At day nine and day 12, the top half of the medium was aspirated and replaced with 0.5 mL of fresh Stemline II medium containing 50 ng/mL SCF and FLT3L. At day 15, floating cells were gently harvested, filtered with a cell strainer, and co-cultured on OP9-DLL4 (kindly provided by Dr. Igor Slukvin) monolayer (2×10⁴ cells/mL) in NK cell differentiation medium: α-MEM medium supplemented with 20% FBS, 5 ng/mL IL-7, 5 ng/mL FTL3L, 25 ng/mL SCF, 5 ng/mL IL-15, and 35 nM UM171. NK cell differentiation medium was changed every three days, and floating cells were transferred onto fresh OP9-DLL4 monolayer every 6 days.

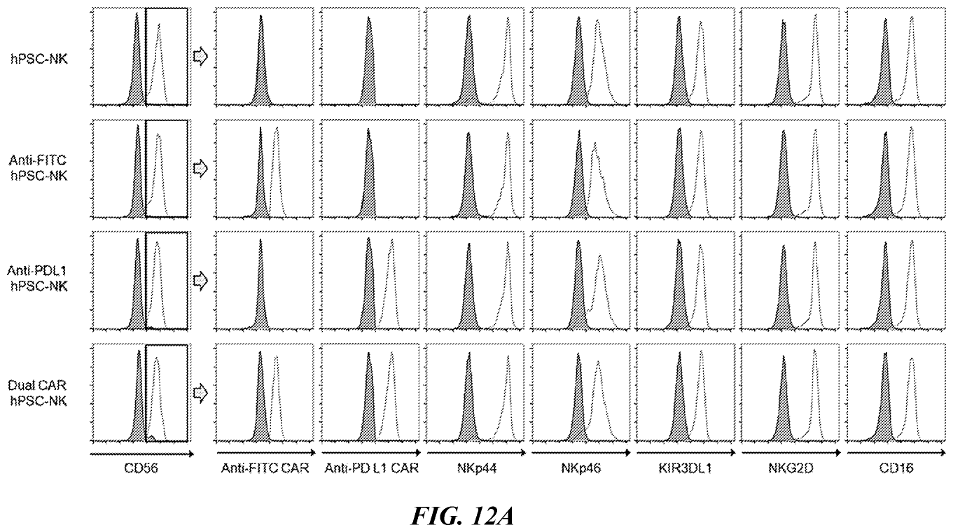

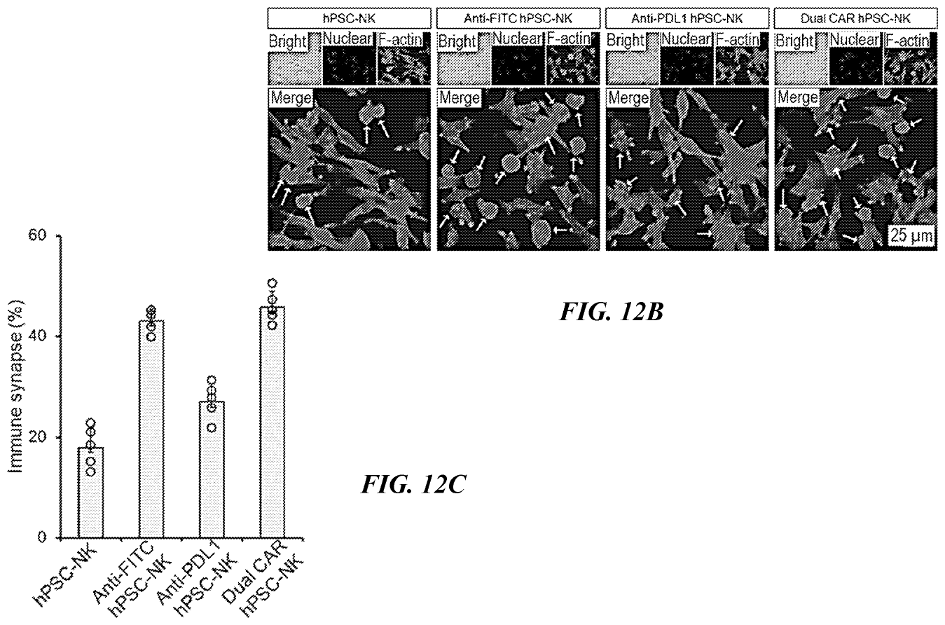

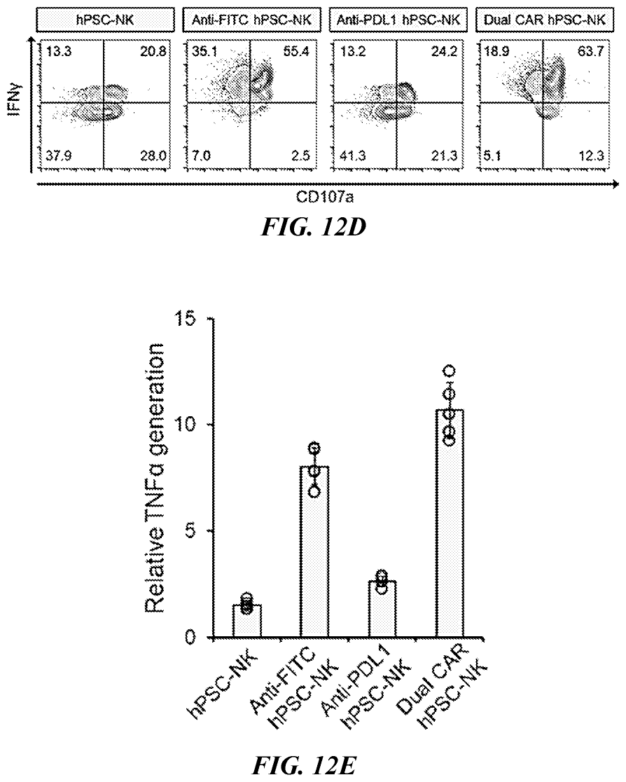

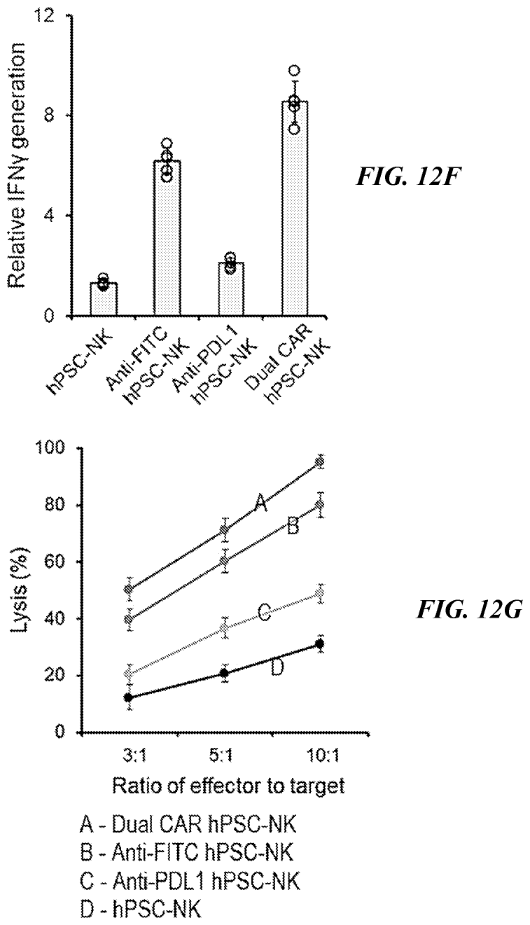

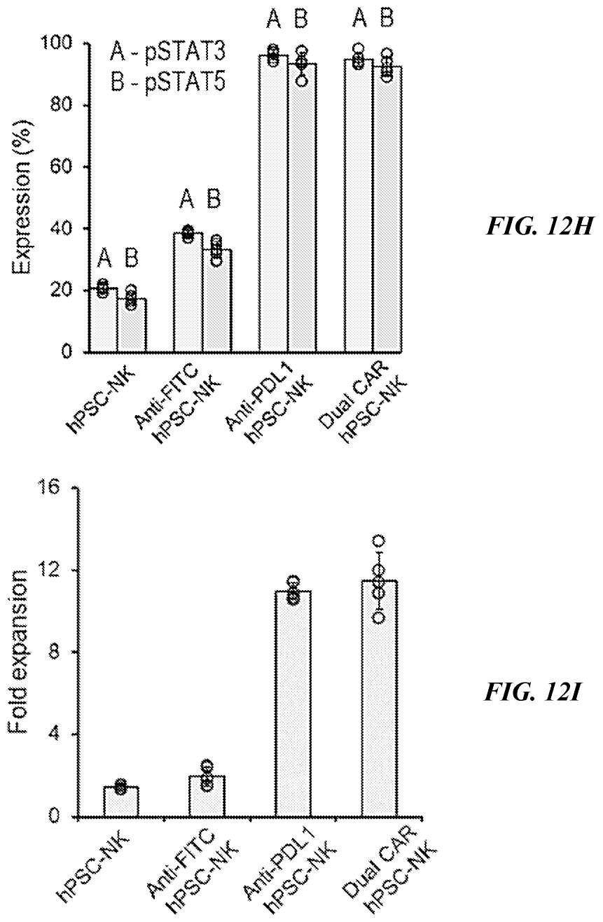

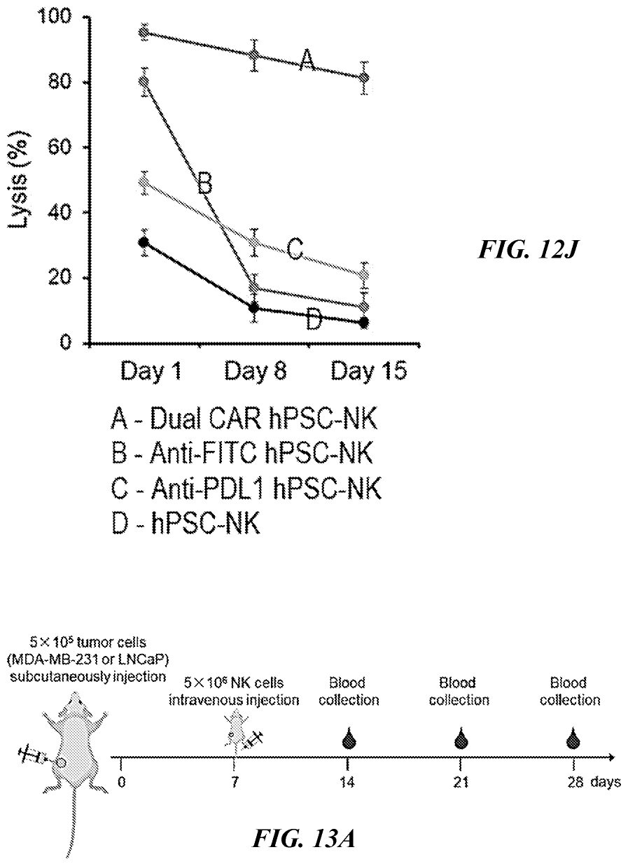

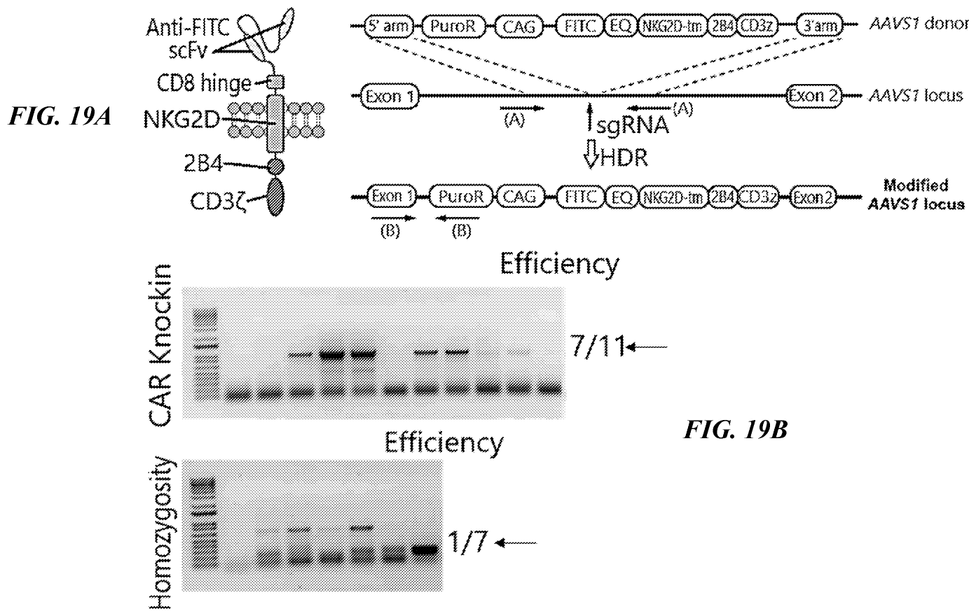

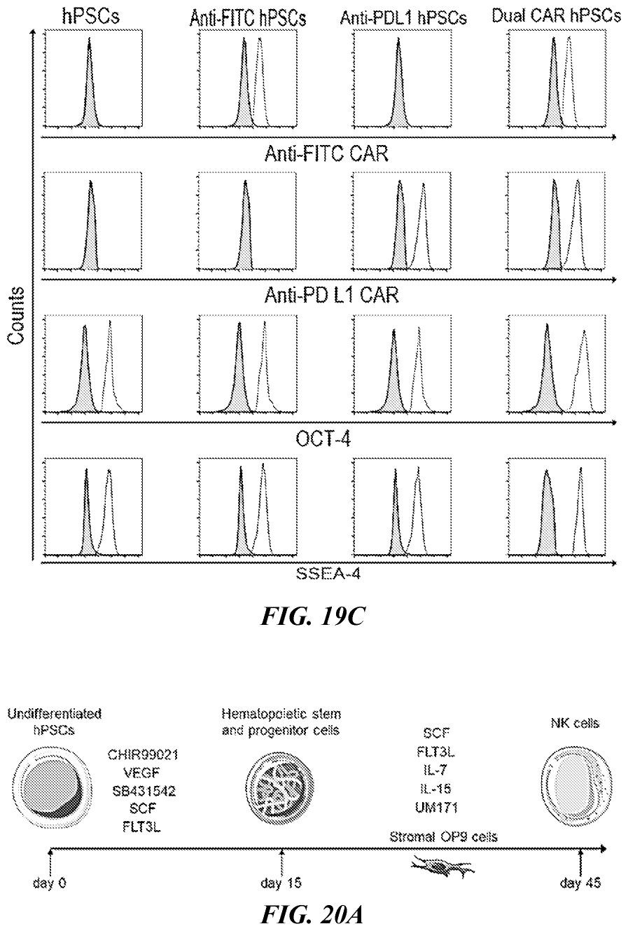

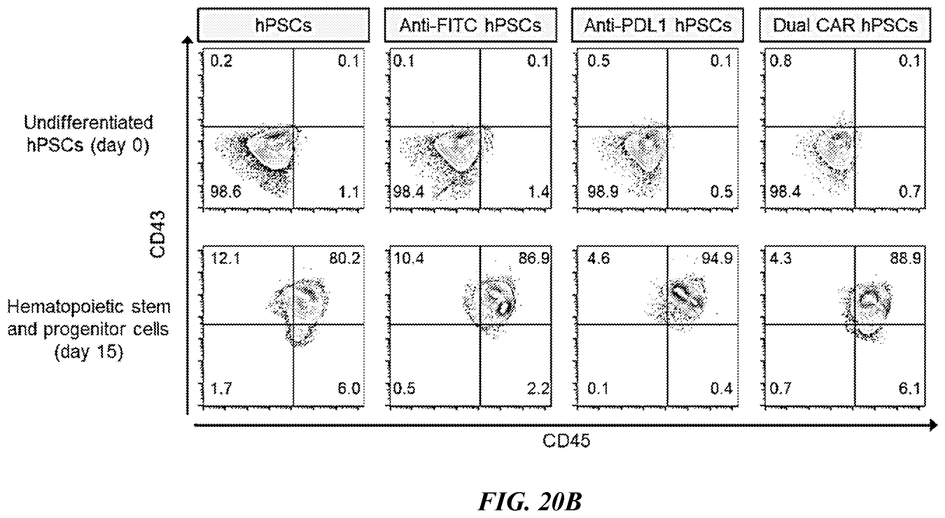

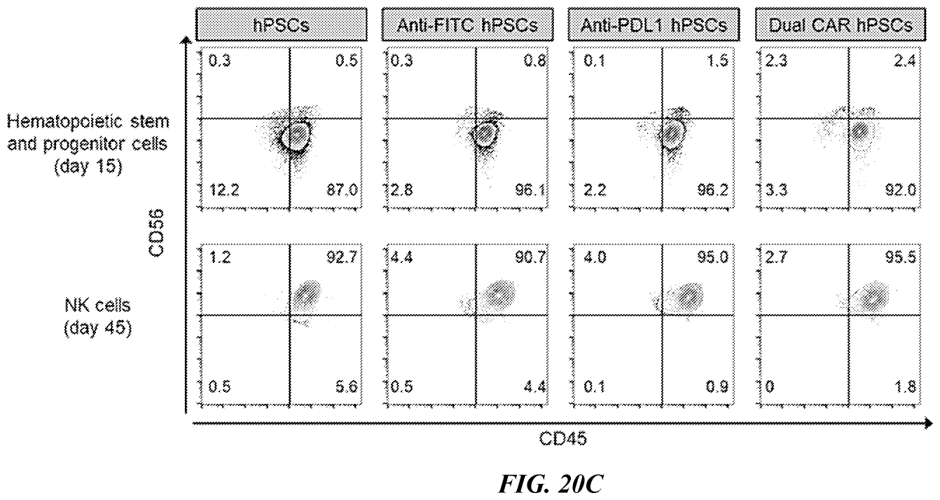

The universal anti-FITC CAR was knocked into the AAVS1 safe harbor locus via CRISPR/Cas9-mediated homologous recombination (FIGS. 19A-19B) and led to robust CAR-expressing hPSCs (FIG. 19C). Notably, engineered hPSCs retained high level expression of pluripotency markers, including stage-specific embryonic antigen-4 (SSEA-4) and octamer-binding transcription factor 4 (OCT-4) (FIG. 19C). To determine the effect of CAR expression on NK cell differentiation, genetically modified hPSCs were subjected to hematopoietic and NK cell differentiation using stage-specific morphogens (FIG. 20A). High purity of CD45+CD43+ hematopoietic stem and progenitor cells (HSPCs) (FIG. 20B) and CD56+CD45+ NK cells (FIG. 20C) were successfully generated from wild-type or CAR-expressing hPSCs. The resulting hPSC-derived NK cells also expressed high levels of typical NK cell surface markers, including CD16, KID3DLT, NKp46, NKG2D, and NKp44 (FIG. 12A).

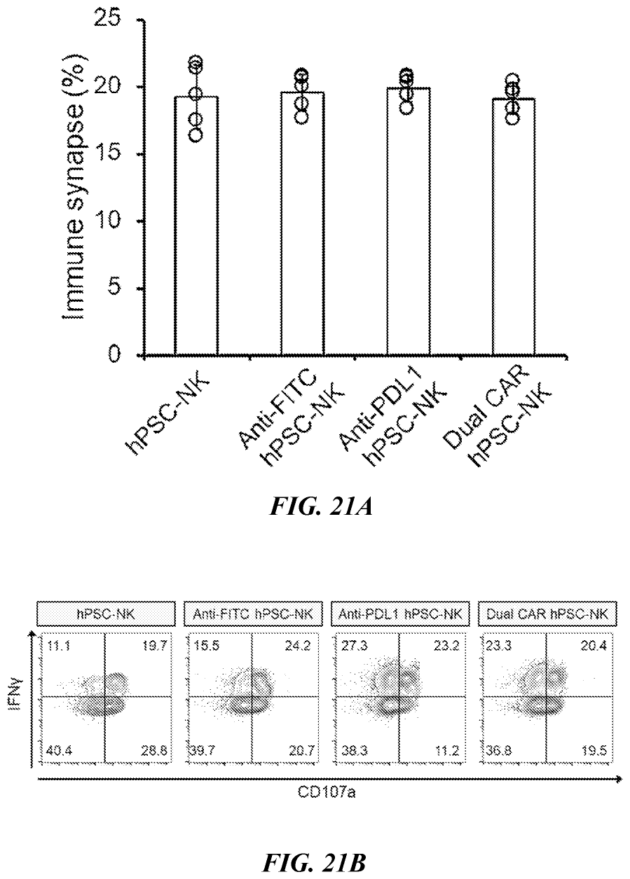

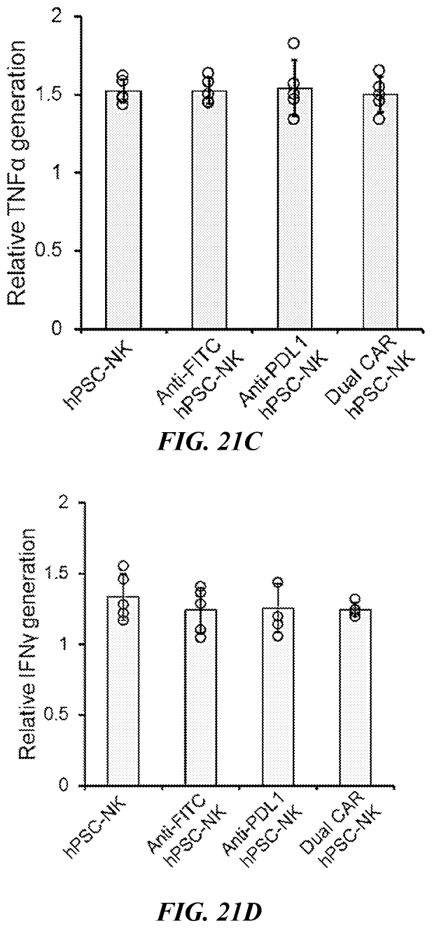

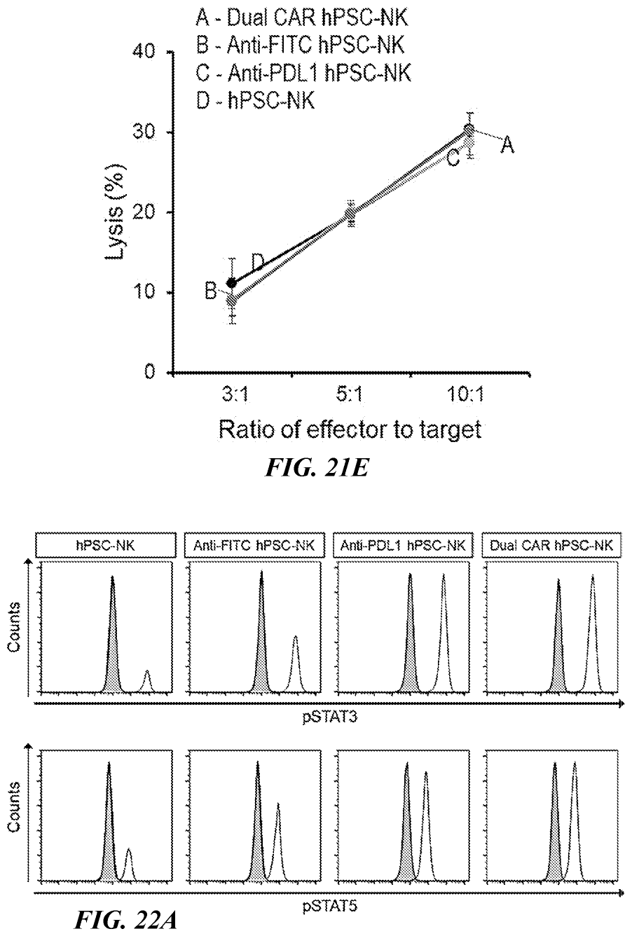

To determine their antitumor cytotoxicity, CAR-expressing hPSC-derived NK cells were co-cultured with MDA-MB-231 cells in the presence of 10 nM FITC-folate. As compared to wild-type hPSC-NK cells, more immunological synapses were formed between CAR-engineered NK cells within two hours (FIG. 12B), and dual CAR-NK cells formed most immunological synapses with tumor cells (FIG. 12C), whereas all hPSC-derived NK cells showed similar and less immunological synapse formation ability against FRα-PD L1− LNCaP prostate cancer cells (FIG. 21A), demonstrating the high specificity of these CARs to the targeted tumor antigens. In response to MDA-MB-231 tumor cells, CAR-NK cells expressed more IFNγ and CD107a (FIG. 12D) and released more cytotoxic granule TNFα and IFNγ (FIGS. 12E-12F). As expected, dual CAR-NK cells expressed most IFNγ and CD107a, and released the most cytotoxic granules, whereas all tested NK cells expressed low levels of IFNγ and CD107a and released low amounts of cytotoxic granules upon FRα-PD-L1− LNCaP cell stimulation (FIGS. 21B-21D). The tumor-killing ability of different hPSC-NK cells was assessed and demonstrated that dual CAR-NK cells displayed superior anti-MDA-MB-231 cytotoxicity as compared to wild-type, anti-FITC CAR, and anti-PD-L1 CAR NK cells (FIG. 12G), whereas all hPSC-derived NK cells displayed similar and low cytotoxicity against LNCaP tumor cells (FIG. 21E).

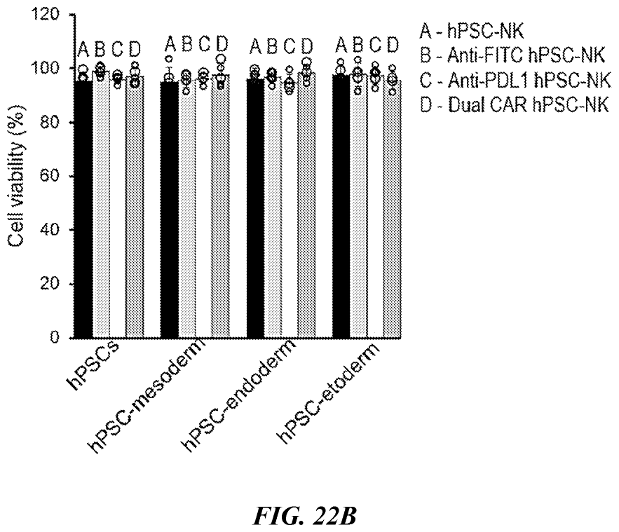

The antigen-responsive proliferation ability of various hPSC-NK cells was investigated. Upon PD-L1+ MDA-MB-231 cell stimulation, hPSC-derived CAR-NK cells upregulated expression levels of phosphorylated STAT3 (pSTAT3) and pSTAT5 (FIG. 22A). Single antigen-targeting anti-PD-L1 and dual CAR-NK cells exhibited highest expression levels of pSTAT3 and pSTAT5 (FIG. 12H) and achieved highest cell expansion (FIG. 12I). The antitumor cytotoxicity and persistence of CAR-NK cells in a continuous antigen exposure model was investigated. While similar strong initial anti-MDA-MB-231 cytotoxicity was observed in anti-FITC and dual CAR NK cells at day 1 (FIG. 12J), anti-FITC CAR-NK cells significantly reduced tumor-killing ability as antigen exposure time increase (day 8 and 15), whereas dual CAR-NK cells still exhibited excellent anti-tumor ability and persistence at day 15. Importantly, all CAR-expressing hPSC-derived NK cells did not kill normal H9 hPSCs and hPSCs-derived somatic cells (FIG. 22B), demonstrating their safety in future clinical applications.

### Example 8

### Dual CAR-hPSC-NK Cells have Improved Antigen-Responsive Persistence In Vivo

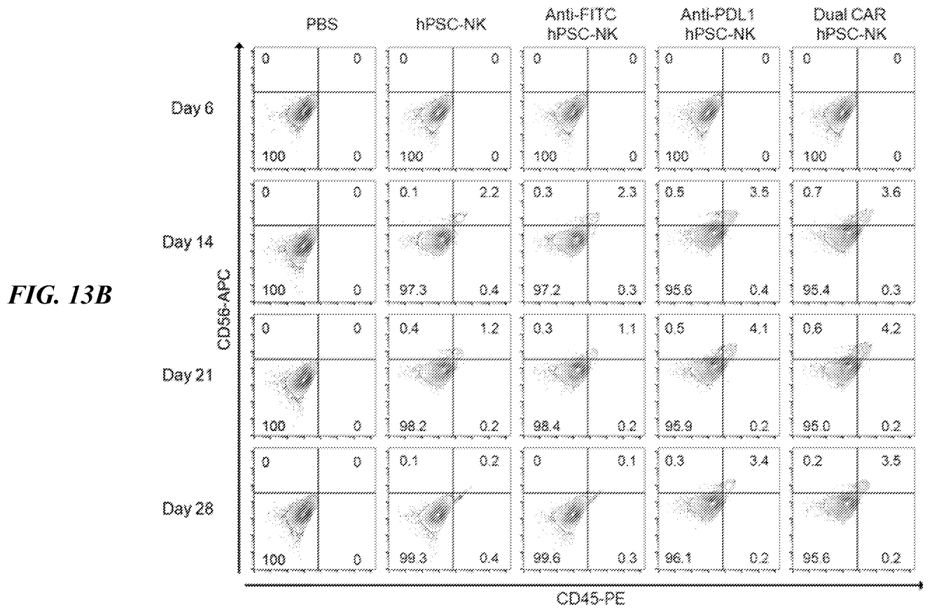

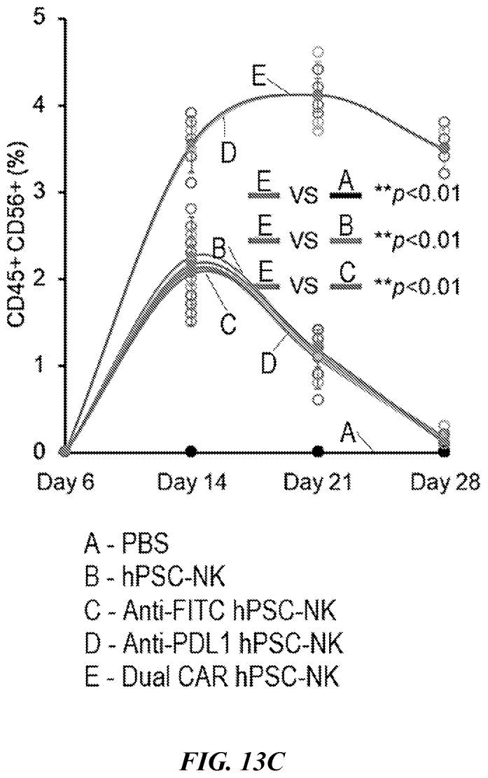

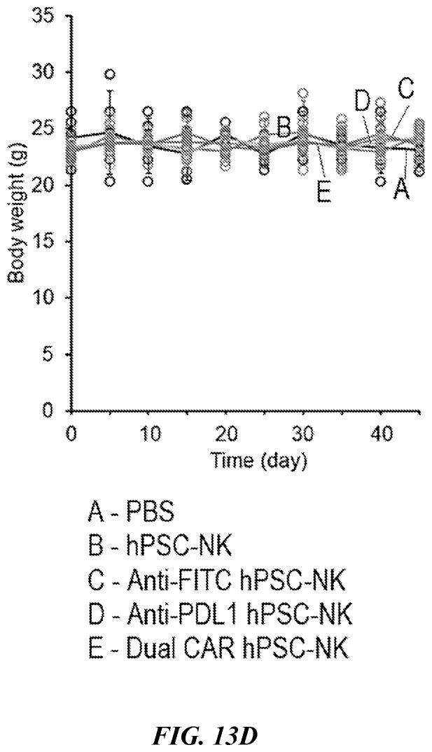

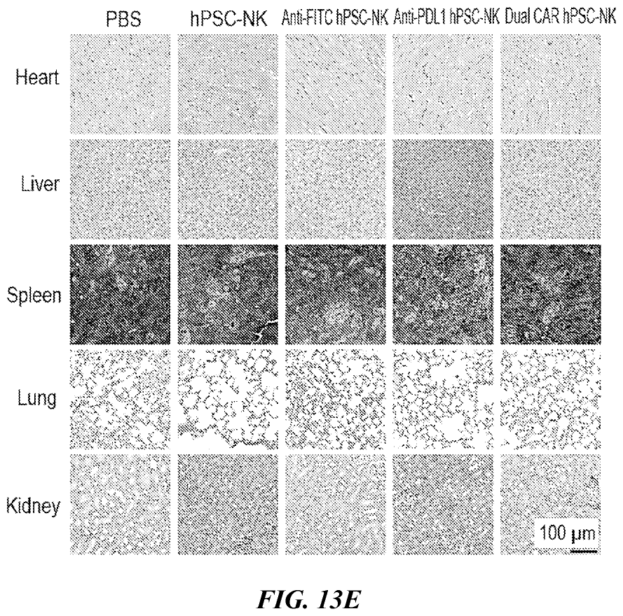

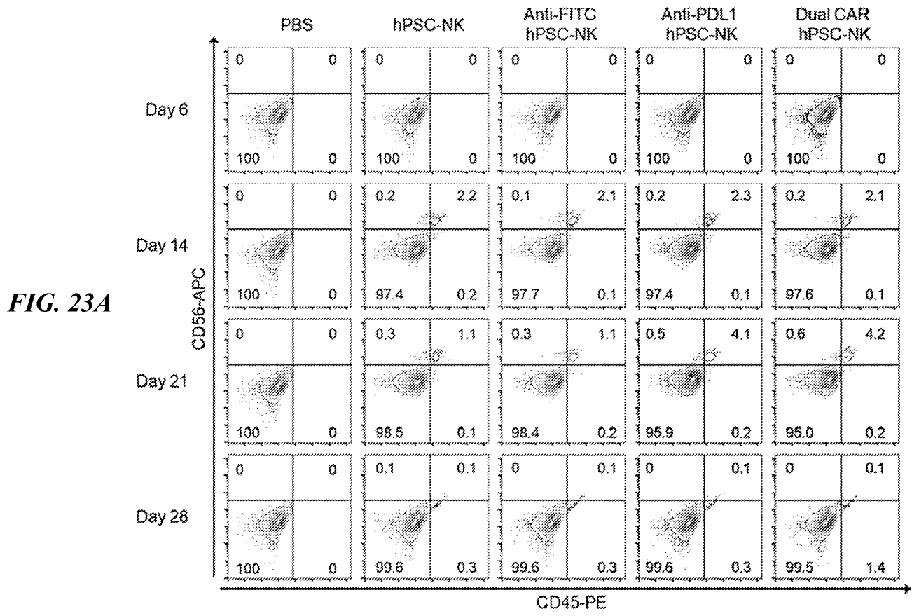

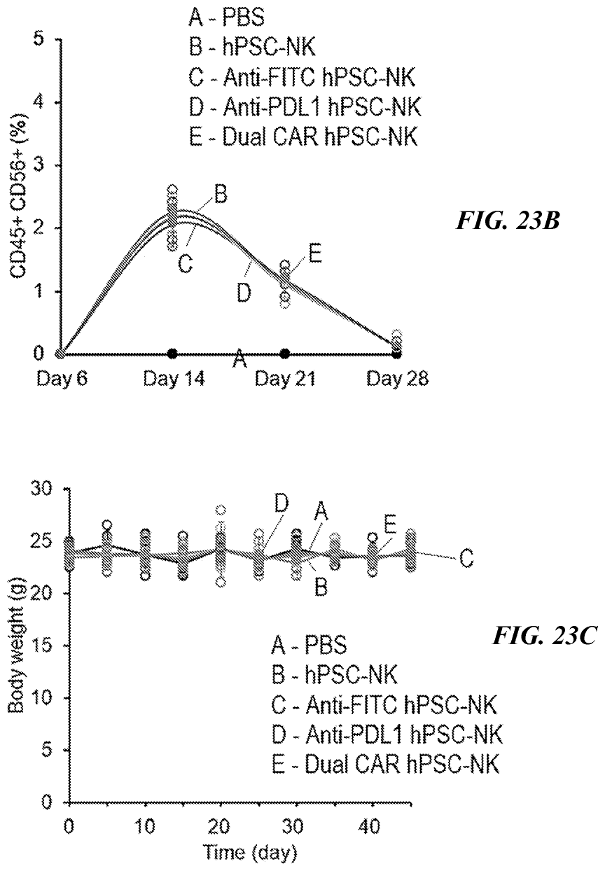

Systemic administration or ectopic expression of interleukin-15 (IL-15) has been used to improve in vivo persistence of CAR-NK cells, although it may lead to abnormal cell proliferation or even leukemia transformation. Ma et al. (2021), supra; Liu et al. (2018), supra; Du et al. (2021), supra; Mishra et al. (2012), supra. To determine the effect of anti-PD-L1 CAR on the proliferation and persistence of hPSC-NK cells, NRG mice engrafted with 5×10⁵ PD-L1-expressing MDA-MB-231 breast cancer cells or PD-L1-rare LNCaP cells (FIGS. 13A and 23) were treated with intravenous infusion of 5×10⁶ different hPSC-derived NK cells or PBS 7 days after tumor cell injection. Host blood was collected at day 6, 14, 21, and 28 for NK cell analysis, and significantly higher NK cell numbers were detected in the anti-PD-L1 and dual CAR NK groups in the MDA-MB-231 mouse xenograft tumor model than in other groups (FIGS. 13B-13C). As expected, low NK cells were detected in all experimental groups of LNCaP mouse xenograft model (FIGS. 23A-23B), highlighting the specificity of anti-PD-L1 CAR and its capacity to enhance persistence of NK cells in vivo.

The biocompatibility of hPSC-derived CAR-NK cells was also evaluated by monitoring the body weight of host mice, and there was no significant body weight loss across all tested experimental groups (FIGS. 13D and 23C), indicating the minimal systemic toxicity and high biocompatibility of hPSC-derived NK cells. Histological analysis of major organs sliced from host mice at day 30 showed that adoptive NK cells did not cause any observable abnormality or damage in heart, liver, spleen, lung, and kidney (FIG. 13E), confirming the biocompatibility of the hPSC-derived NK cells.

### Example 9

### Dual CAR-hPSC-NK Cells have Improved Antitumor Activities in Tumor Rechallenge Models

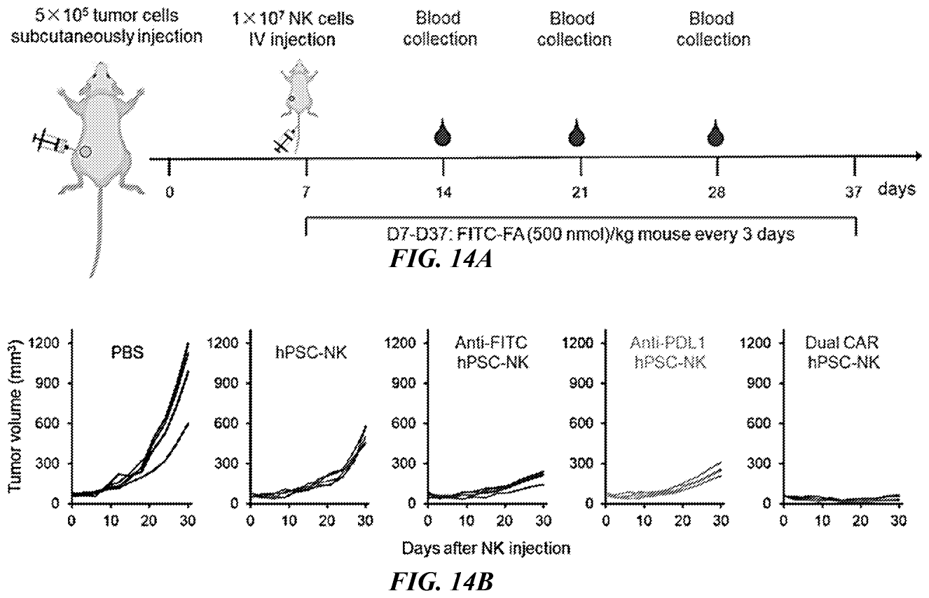

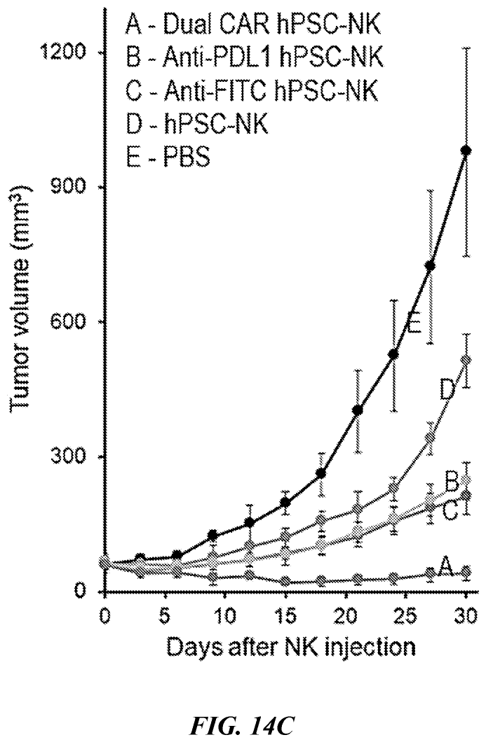

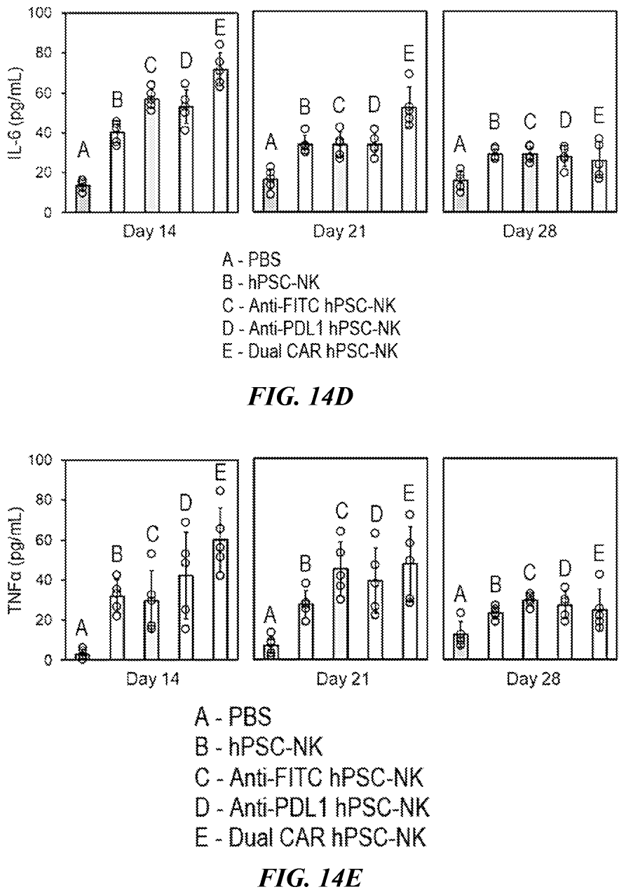

To evaluate antitumor activities of different hPSC-NK cells in vivo, the cytotoxicity of MDA-MB-231 cells was tested in a mouse xenograft model. All the mouse experiments were approved by the Purdue Animal Care and Use Committee (PACUC). Briefly, the immunodeficient NOD.Cg-RAG^{1tm1Mom}IL2rg^{tm1Wjl}/SzJ (NRG) mice were bred and maintained by the Biological Evaluation Core at the Purdue University Center for Cancer Research. MDA-MB-231 cells (5×10⁵ tumor cells/per mouse) were implanted subcutaneously. When the tumor size reached ˜100 mm³, NK cells and FITC-folate were intravenously injected (single injection of 1×10⁷ NK cells seven days after tumor inoculation (FIG. 14A)). Mice were maintained on a folic acid-deficient diet (TD.95247, Envigo RMS, LLC, Indianapolis, IN) to reduce the level of folic acid in mice to a physiological level found in humans.

Tumors were measured every five days with calipers, and the tumor volume was calculated according to the equation: tumor volume=LxW²×½, where L is the longest axis of the tumor and W is the axis perpendicular to L. Mouse blood was also collected for NK cell and cytokine release (TNFα and IL-6) analysis, and systemic toxicity was monitored by measuring body weight loss of experimental mice.

As compared to the tumor-bearing mice treated with PBS, administration of hPSC-NK cells significantly reduced tumor burden (FIGS. 14B-14C). As expected, dual CAR hPSC-NK cells displayed higher anti-tumor cytotoxicity than wild-type or other CAR-expressing NK cells. We next measured human cytokine production release in the plasma of different experimental mouse groups, including TNFα and IL-6. All non-PBS experimental groups released detectable TNFα and IL-6 in the plasma from day 14 to day 28, and dual CAR hPSC-NK cells maintained highest levels of both cytokines (FIGS. 14D-14E), which were eventually decreased in host mice, indicating a reduced risk of cytokine release syndrome.

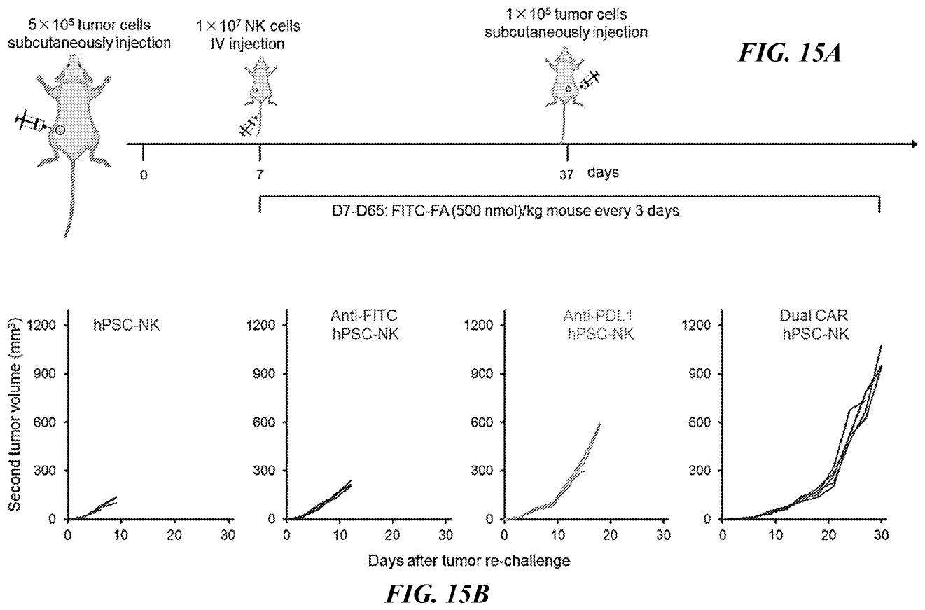

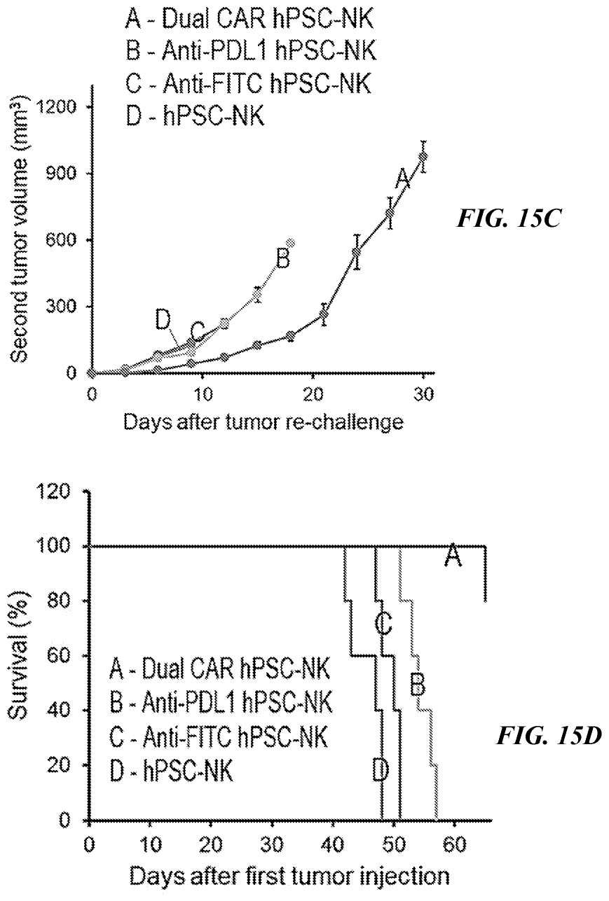

Given the promising in vivo performance of our hPSC-derived NK cells, MDA-MB-231 tumor cells were re-inoculated to construct a tumor rechallenge model for the investigation of their memory-like behavior (FIG. 15A). Compared with other experimental groups, dual CAR hPSC-NK cells significantly reduced tumor burden (FIGS. 15B-15C) and prolonged the survival of tumor-bearing mice (FIG. 15D). The data indicate that the combination of tumor microenvironment responsive anti-PD-L1 and programmable anti-FITC CARs significantly enhances in vivo persistence and antitumor activities of hPSC-derived NK cells, endowing them with a memory-like capacity for improved immunotherapy.

1. A population of natural killer (NK) cells derived from human pluripotent stem cells (hPSCs) and engineered to:
overexpress transcription factor ID2, NFIL3, and/or SPI1; and
express an anti-programmed death ligand 1 (PD-L1) chimeric antigen receptor (CAR) and an anti-fluorescein isothiocyanate (FITC) CAR.

2. (canceled)

3. The population of NK cells of claim 1, wherein the anti-PD-L1 CAR and/or anti-FITC CAR comprises a truncated cytoplasmic domain from interleukin-2 (IL-2) receptor β-chain, a STAT3-binding tyrosine-X-X-glutamine (YXXQ) motif, or both.

4. The population of NK cells of claim 1, wherein the anti-PD-L1 CAR and/or the anti-FITC CAR comprises NK cell-Fc receptor transmembrane and intracellular signaling domains.

5. (canceled)

6. The population of NK cells of claim 1, wherein the overexpression of the transcription factor(s) is inducible.

7. (canceled)

8. The population of NK cells of claim 1, which expresses at least one NK cell-specific marker.

9. The population of NK cells of claim 8, wherein the at least one NK cell-specific marker is NKp44, NKp46, KIR3DL1, NKG2D, or any combination thereof.

10. The population of NK cells of claim 1, wherein the hPSCs comprise human embryonic stem cells (hESCs) and/or induced pluripotent stem cells (iPSCs).

11-26. (canceled)

27. The population of NK cells of claim 1 formulated in combination with
a pharmaceutically acceptable carrier and/or diluent.

28. The population of NK cells of claim 27, further comprising a pharmaceutically acceptable excipient.

29. (canceled)

30. A method of treating cancer in a subject comprising administering to the subject a first therapy comprising a therapeutically effective amount of:
a population of the NK cells of claim 1;
whereupon the subject is treated for cancer.

31. The method of claim 30, further comprising administering to the subject a conjugate comprising FITC linked to a ligand that binds folate receptor α (FRα), prostate-specific membrane antigen (PSMA), or carbonic anhydrase IX (CAIX).

32. (canceled)

33. The method of claim 31, wherein the ligand binds PSMA and is DUPA.

34. (canceled)

35. The method of claim 30, further comprising administering a second therapy to the subject.

36. The method of claim 35, wherein the second therapy comprises a therapeutically effective amount of chemotherapy, radiotherapy, or the surgical removal of cancerous cells from the subject.

37-38. (canceled)

39. The method of claim 30, wherein administering the first therapy comprises a delivery route selected from the group consisting of intravenous, intraperitoneal, intramuscular, intradermal, subcutaneous, intrathecal, intraosseous, and a combination of any of the foregoing.

40. The method of claim 35, wherein the second therapy comprises a chemotherapy, radiotherapy, or both.

41. The method of claim 35, further comprising imaging a cancer in the subject prior to or during administering the first and/or second therapies.

42. The method of claim 35, wherein the first and second therapies are administered sequentially and/or alternatively.

43. A method of producing the population of natural killer (NK) cells of claim 1, the method comprising differentiating a population of human pluripotent stem cells (hPSCs) to NK cells, the population of hPSCs engineered to overexpress transcription factor ID2, NFIL3, and/or SPI1.

44. The method of claim 43, wherein the population of hPSCs is engineered to express an anti-programmed death ligand 1 (PD-L1) chimeric antigen receptor (CAR) and an anti-fluorescein isothiocyanate (FITC) CAR.

45-49. (canceled)

50. The method of claim 43, wherein the overexpression of the transcription factor(s) is inducible.


SEQUENCE LISTING (4 sequences)

SEQ ID NO: 1 - DNA, 25 bp, synthetic construct
  source 1..25: mol_type = other DNA
ctgtttccccttcccaggcaggtcc

SEQ ID NO: 2 - DNA, 25 bp, synthetic construct
  source 1..25: mol_type = other DNA
tcgtcgcgggtggcgaggcgcaccg

SEQ ID NO: 3 - DNA, 20 bp, synthetic construct
  source 1..20: mol_type = other DNA
cggttaatgtggctctggtt

SEQ ID NO: 4 - DNA, 20 bp, synthetic construct
  source 1..20: mol_type = other DNA
gagagagatggctccaggaa